(19) United States
(12) Patent Application Publication
(10) Pub. No.: US 2007/0253950 A1
(43) Pub. Date: Nov. 1, 2007

(54) Methods for Preventing and Treating Amyloidogenic Diseases

(75) Inventor: Jack Steven Jacobsen, Ramsey, NY (US)

Correspondence Address: PILLSBURY WINTHROP SHAW PITTMAN, LLP, P.O. BOX 10500, MCLEAN, VA, 22102, US

(73) Assignee: Wyeth, Madison, NJ (US)

(21) Appl. No.: 11/689,501

(22) Filed: Mar. 21, 2007

Related U.S. Application Data
(60) Provisional application No. 60/784,575, filed on Mar. 21, 2006.
(60) Provisional application No. 60/895,303, filed on Mar. 16, 2007.

(51) Int. Cl. A61K 39/395; A61P 25/28

(52) U.S. Cl. 424/133.1; 424/139.1

(57) ABSTRACT

A method for treating a disease or disorder characterized by amyloid deposit of A-beta comprising administering to the subject a therapeutically effective amount of an antibody that binds specifically to RAGE and inhibits the binding of a RAGE binding partner.

### CROSS-REFERENCES TO RELATED APPLICATIONS

Priority is claimed under 35 U.S.C. §119(e) of U.S. Provisional Patent Application No. 60/895,303, filed Mar. 16, 2007, and U.S. Provisional Patent Application No. 60/784,575, filed Mar. 21, 2006, the contents of both of which are incorporated herein by reference in their entirety.

### FIELD OF THE INVENTION

The present invention generally relates to antibodies and fragments thereof that bind specifically to a receptor for advanced glycation endproducts (RAGE), to methods in which such antibodies and fragments thereof are administered to human patients and non-human mammals to treat or prevent RAGE-related diseases and disorders.

### BACKGROUND OF THE INVENTION

Alzheimer's disease (AD) is a progressive, ultimately fatal, neurodegenerative disorder that affects primarily the elderly. It is the most common form of dementia and is typically associated with the gradual loss of cognition (memory, reasoning, orientation and judgment) and the progression of a number of behavioral disorders. The disease tends to fall into two categories: late onset, which occurs in old age (65+ years) and early onset, which develops well before the senile period, i.e., between 35 and 60 years. In both types of disease, the pathology is the same but the abnormalities tend to be more severe and widespread in cases beginning at an earlier age. The disease is characterized by at least two types of lesions in the brain, neurofibrillary tangles and senile plaques. Neurofibrillary tangles are intracellular deposits of microtubule associated tau protein consisting of two filaments twisted about each other in pairs. Senile plaques (i.e., amyloid plaques) are areas of disorganized neuropil threads up to 150 μm across with extracellular amyloid deposits at the center which are visible by microscopic analysis of sections of brain tissue. The accumulation of amyloid plaques within the brain is also associated with Down's syndrome and other cognitive disorders, such as serum amyloid A (SAA) amyloidosis and spongiform encephalopathies.

A peptide termed β-amyloid peptide or Aβ is the principal constituent of amyloid plaques and is believed to play a fundamental role in AD pathogenesis. Aβ is a hydrophobic 4-kDa internal fragment of 39-43 amino acid residues of a larger transmembrane glycoprotein protein termed amyloid precursor protein (APP). As a result of proteolytic processing of APP by β-secretase and γ-secretase, Aβ is primarily found in both a short form, 40 amino acids in length, and a long form, ranging from 42-43 amino acids in length. Accumulation of amyloid plaques in the brain eventually leads to neuronal cell death. The deficits and physical symptoms characteristic of Alzheimer's disease (AD) are believed to result, at least in part, from the neural deterioration caused by the neurotoxic effects of Aβ. Reduction of Aβ levels by inhibition of Aβ production or enhancement of Aβ clearance is a widely recognized disease modifying strategy for Alzheimer's disease.

Several mutations within the APP protein have been correlated with the presence of Alzheimer's disease. Examples of such mutations include valine⁷¹⁷ to isoleucine, glycine or phenylalanine, and a double mutation changing lysine⁵⁹⁵-methionine⁵⁹⁶ to asparagine⁵⁹⁵-leucine⁵⁹⁶. Such mutations are thought to cause Alzheimer's disease by increased or altered processing of APP to Aβ, particularly processing of APP to increased amounts of the long form of Aβ (i.e., Aβ1-42 and Aβ1-43). Mutations in other genes, such as the presenilin genes, PS1 and PS2, are thought indirectly to affect processing of APP to generate increased amounts of long form Aβ.

Mouse models have been used successfully to determine the significance of amyloid plaques in Alzheimer's (Games et al., 1995, Nature, 373:523; Johnson-Wood et al., 1997, Proc. Natl. Acad. Sci. USA, 94:1550). In particular, when PDAPP transgenic mice, which express a mutant form of human APP and develop Alzheimer's disease at a young age, are injected with the long form of Aβ, they display both a decrease in the progression of Alzheimer's and an increase in antibody titers to the Aβ peptide (Schenk et al., 1999, Nature, 400:173). The observations discussed above indicate that Aβ, particularly in its long form, is a causative element in Alzheimer's disease.

The Aβ peptide can exist in solution and can be detected in CNS (e.g., CSF) and plasma. Under certain conditions, soluble Aβ is transformed into fibrillary, toxic, β-sheet forms found in neuritic plaques and cerebral blood vessels of patients with AD. Treatments involving immunization with monoclonal antibodies against Aβ have been investigated. Both active and passive immunization have been tested in mouse models of AD. Active immunization resulted in some reduction in plaque load in the brain, but only when administered nasally. Passive immunization of PDAPP transgenic mice has also been investigated (Bard, et al., 2000, Nature Med., 6:916-19). Two mechanisms are proposed for effective clearance, i.e., central degradation and peripheral degradation. The central degradation mechanism relies on antibodies being able to cross the blood-brain barrier, bind to plaques, and induce clearance of pre-existing plaques. Clearance has been shown to be promoted through an Fc-receptor-mediated phagocytosis (Bard, et al., supra). The peripheral degradation mechanism of Aβ clearance relies on a disruption of the dynamic equilibrium of Aβ between brain, CSF, and plasma upon administration of antibody, leading to transport of Aβ from one compartment to another. Centrally derived Aβ is transported into the CSF and the plasma where it is degraded. Recent studies have suggested that soluble and unbound Aβ are involved in the memory impairment associated with AD, even without reduction in amyloid deposition in the brain. (Dodel, et al., 2003, The Lancet, 2:215).

The receptor for advanced glycation endproducts (RAGE) is a multi-ligand cell surface member of the immunoglobulin super-family. RAGE consists of an extracellular domain, a single membrane-spanning domain, and a cytosolic tail. The extracellular domain of the receptor consists of one V-type immunoglobulin domain followed by two C-type immunoglobulin domains. RAGE also exists in a soluble form (sRAGE). RAGE is expressed by many cell types, e.g., endothelial and smooth muscle cells, macrophages and lymphocytes, in many different tissues, including lung, heart, kidney, skeletal muscle and brain. Expression is increased in chronic inflammatory states such as rheumatoid arthritis and diabetic nephropathy. Although its physiologic function is unclear, it is involved in the inflammatory response and may have a role in diverse developmental processes, including myoblast differentiation and neural development.

RAGE is an unusual pattern-recognition receptor that binds several different classes of endogenous molecules leading to various cellular responses, including cytokine secretion, increased cellular oxidant stress, neurite outgrowth and cell migration. RAGE has been shown to have an active, pathogenic role in a wide range of amyloidogenic diseases and disorders.

There exists the need for new therapies and reagents for the treatment of Alzheimer's disease and other amyloidogenic diseases.

### SUMMARY OF THE INVENTION

The present invention provides methods of treating a subject having a disease or disorder characterized by amyloid deposit of A□ by administering therapeutically effective amount of an antibody that binds specifically to RAGE (i.e., anti-RAGE antibodies) and inhibits the binding of a RAGE binding partner. The diseases or disorders treatable by the disclosed methods may be characterized by amyloid deposit of A□in brain, such as occurs in Alzheimer's disease. Anti-RAGE antibodies as described herein may also be used to inhibit or reduce accumulation of amyloid deposit of A□ in a subject, to inhibit or reduce neurodegeneration in a subject, to inhibit or reduce cognitive decline in a subject, and/or to improve cognition in a subject.

### BRIEF DESCRIPTION OF THE DRAWINGS

FIGS. 1A-1C show aligned amino acid sequences of RAGE of mouse, rat, rabbit (2 isoforms), baboon, cynomolgus monkey, and human (SEQ ID NOs: 3, 14, 11, 13, 7, 9, 1).

FIG. 2 is a graph of data from direct binding ELISA that demonstrate binding of XT-H2 to hRAGE with EC50 of 90 pM and binding of XT-M4 to hRAGE-Fc with EC50 of 300 pM.

FIG. 3 is a graph of data from direct binding ELISA analysis that demonstrate binding of antibodies XT-M4 and XT-H2 to the hRAGE V-domain-Fc of with EC50 of 100 pM.

FIG. 4 is graph of data from ligand competition ELISA binding assays showing the ability of XT-H2 and XT-M4 to block the binding of HMG1 to hRAGE-Fc.

FIG. 5 is a graph of data from antibody competition ELISA binding assays showing that XT-H2 and XT-M4 share a similar epitope and bind to overlapping sites on human RAGE.

FIG. 6 shows aligned amino acid sequences of the heavy chain variable regions of murine anti-RAGE antibodies XT-H1, XT-H2, XT-H3, XT-H5 and XT-H7, and of rat anti-RAGE antibody XT-M4 (SEQ ID NOs: 18, 21, 24, 20, 26, 16).

FIG. 7 shows aligned amino acid sequences of the light chain variable regions of murine anti-RAGE antibodies XT-H1, XT-H2, XT-H3, XT-H5 and XT-H7, and of rat anti-RAGE antibody XT-M4 (SEQ ID NOs: 19, 22, 25, 23, 27, 17).

FIG. 8 shows the nucleotide sequence of cDNA encoding baboon RAGE (SEQ ID NO: 6).

FIG. 9 shows the nucleotide sequence of cDNA encoding cynomolgus monkey RAGE (SEQ ID NO: 8).

FIG. 10 shows the nucleotide sequence of cDNA encoding rabbit RAGE isoform 1 (SEQ ID NO:10).

FIG. 11 shows the nucleotide sequence of cDNA encoding rabbit RAGE isoform 2 (SEQ ID NO: 12).

FIGS. 12A-12E show the nucleotide sequence of cloned baboon genomic DNA encoding baboon RAGE (clone 18.2) (SEQ ID NO: 15).

FIG. 13 presents four graphs showing the abilities of chimeric XT-M4 antibody and rat antibody XT-M4 to block the binding of RAGE ligands HMGB1, amyloid β 1-42 peptide, S100-A, and S100-B to hRAGE-Fc, as determined by competition ELISA binding assay.

FIG. 14 presents graphs showing the ability of chimeric XT-M4 to compete for binding to hRAGE-Fc with antibodies XT-M4 and XT-H2, as determined by antibody competition ELISA binding assay.

FIG. 15 depicts IHC-staining of lung tissues of cynomologus monkey, rabbit, and baboon, showing that the XT-M4 binds to endogenous cell-surface RAGE in these tissues. Control samples are CHO cells expressing hRAGE contacted by XT-M4, NGBCHO cells that do not express RAGE, and CHO cells expressing hRAGE contacted by a control IgG antibody.

FIG. 16 shows that the rat antibody XT-M4 binds to RAGE in normal human lung and lung of a human with chronic obstructive pulmonary disease (COPD).

FIG. 17 shows amino acid sequences of humanized murine XT-H2 HV region.

FIG. 18 shows amino acid sequences of humanized murine XT-H2 HL region.

FIG. 19 shows amino acid sequences of humanized rat XT-M4 HV region.

FIGS. 20A-20B show amino acid sequences of humanized rat XT-H2 HL region.

FIG. 21 depicts expression vectors used to produce humanized light and heavy chain polypeptides.

FIG. 22 shows ED50 values for the binding of humanized XT-H2 antibodies to human RAGE-Fc as determined by competition ELISA.

FIG. 23 shows kinetic rate constants (kₐ and k_{d}) and association and dissociation constants (Kₐ and K_{d}) for binding of XT-M4 and humanized antibodies XT-M4-V10, XT-M4-V11, and XT-M4-V14 to hRAGE-SA, as determined by BIACORE™ binding assay.

FIG. 24 shows kinetic rate constants (kₐ and k_{d}) and association and dissociation constants (Kₐ and K_{d}) for binding of XT-M4 and humanized antibodies XT-M4-V10, XT-M4-V11, and XT-M4-V14 to mRAGE-SA, as determined by BIACORE™ binding assay.

FIG. 25 shows the nucleotide sequence of a murine XT-H2 VL-VH ScFv construct (SEQ ID NO:51).

FIG. 26 shows the nucleotide sequence of a murine XT-H2 VH-VL ScFv construct (SEQ ID NO: 52).

FIG. 27 shows the nucleotide sequence of a rat XT-M4 VL-VH ScFv construct (SEQ ID NO: 54).

FIG. 28 shows the nucleotide sequence of a rat XT-M4 VH-VL ScFv construct (SEQ ID NO: 53).

FIG. 29 is a graph of ELISA data showing binding to human RAGE-Fc by ScFv constructs of the XT-H2 and XT-M4 anti-RAGE antibodies in either the VL/VH or VH/VL configuration.

FIG. 30 is a graph of ELISA data showing binding to human RAGE-Fc and BSA by ScFv constructs of the XT-H2 and XT-M4 anti-RAGE antibodies in the VL/VH or VH/VL configuration expressed as soluble protein in Escherichia coli. ActRIIb is a non-binding protein expressed from the same vector as a negative control.

FIG. 31 schematically represents the use of PCR to introduce spiked mutations into a CDR of XT-M4.

FIG. 32 shows the nucleotide sequence of the C terminal end of the XT-M4 VL-VH ScFv construct (SEQ ID NO: 56). VH-CDR3 is underlined. Also shown are two spiking oligonucleotides (SEQ ID NOs: 57-58) with a number at each mutation site that identifies the spiking ratio used for mutation at that site. The nucleotide compositions of the spiking ratios corresponding to the numbers are also identified.

FIG. 33 schematically represents the ribosome display vector pWRIL-3. “T7” denotes T7 promotor, “RBS” is the ribosome binding site, “spacer polypeptide” is a spacer polypeptide connecting the folded protein to the ribosome, “Flag-tag” is Flag epitope tag for protein detection.

FIG. 34 schematically represents the phage display vector pWRIL-1.

FIG. 35 schematically represents the combinatorial assembly of VL and VH spiked libraries using the Fab display vector pWRIL-6.

FIG. 36 is a graph of antibody competition ELISA data show increased affinity of the XT-M4 antibody for hRAGE following mutation that removes the glycosylation site at position 52.

FIG. 37 is a graph showing serum concentration of chimeric XT-M4 following a single iv administration to mice.

FIG. 38 is a graph showing the effects of chimeric XT-M4 on memory deficits in the Tg2576 mouse model.

### DETAILED DESCRIPTION OF THE INVENTION

Anti-RAGE Antibodies

The present invention provides antibodies that bind specifically to RAGE, including soluble RAGE and endogenous secretory RAGE, as described herein. Representative anti-RAGE antibodies may comprise at least one of the antibody variable region amino acid sequences shown in SEQ ID NOs: 16-49.

The anti-RAGE antibodies of the invention include antibodies that bind specifically to RAGE and have an amino acid sequence that is identical or substantially identical to any one of SEQ ID NOs: 16-49. An amino acid sequence of an anti-RAGE antibody that is substantially identical is one that has at least 85%, 85%, 87%, 88%, 89%, 90%, 91%, 92%, 93%, 94%, 95%, 96%, 97%, 98%, 99%, 99.5%, or 99.9% identity to any one of SEQ ID NOs: 16-49.

Included in the anti-RAGE antibodies of the invention is an antibody that binds specifically to RAGE, and (a) comprises a light chain variable region selected from the group consisting of SEQ ID NOs: 19, 22, 25, 23, 27 and 17, or (b) comprises a light chain variable region having an amino acid sequence that is at least 90% identical to any one of SEQ ID NOs: 19, 22, 25, 23, 27 and 17, or is a RAGE-binding fragment of an antibody according to (a) or (b).

Also Included in the anti-RAGE antibodies of the invention is an antibody that binds specifically to RAGE, and (a) comprises a heavy chain variable region selected from the group consisting of SEQ ID NOs: 18, 21, 24, 20, 26, and 16, or (b) comprises a heavy chain variable region having an amino acid sequence that is at least 90% identical to any one of SEQ ID NOs: 18, 21, 24, 20, 26, and 16, or is a RAGE-binding fragment of an antibody according to (a) or (b).

Included in the invention is an anti-RAGE antibody that binds specifically to RAGE and:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

The invention includes anti-RAGE antibodies that bind specifically to RAGE-expressing cells in vitro and in vivo, and antibodies that bind to human RAGE with a dissociation constant (Kd) in the range of from at least about 1×10⁻⁷ M to about 1×10⁻¹⁰ M. Also Included are anti-RAGE antibodies of the invention that bind specifically to the V domain of human RAGE, and anti-RAGE antibodies that block the binding of RAGE to a RAGE binding partner (RAGE-BP).

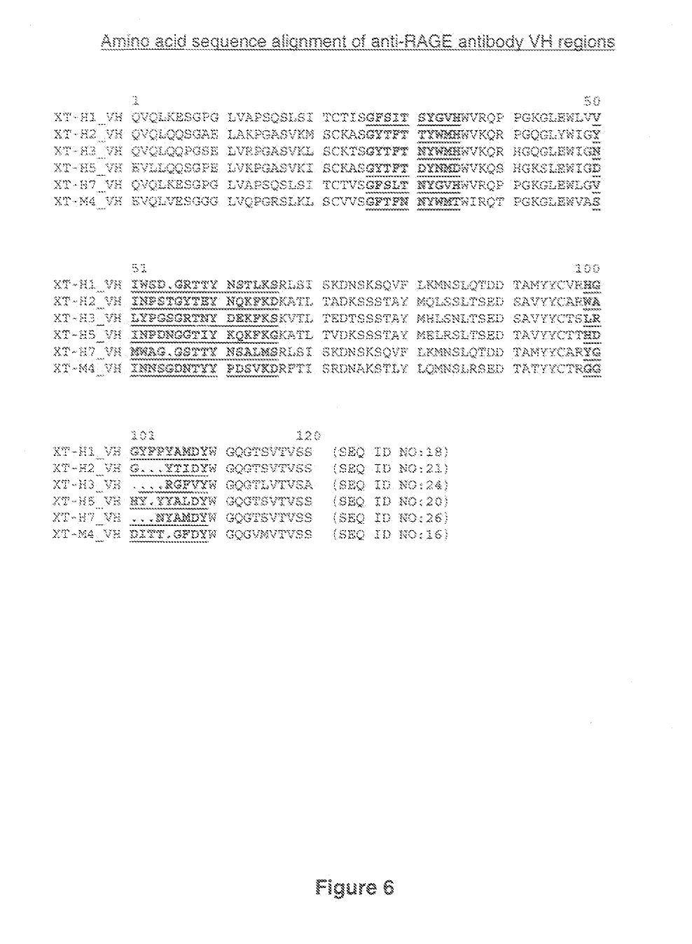

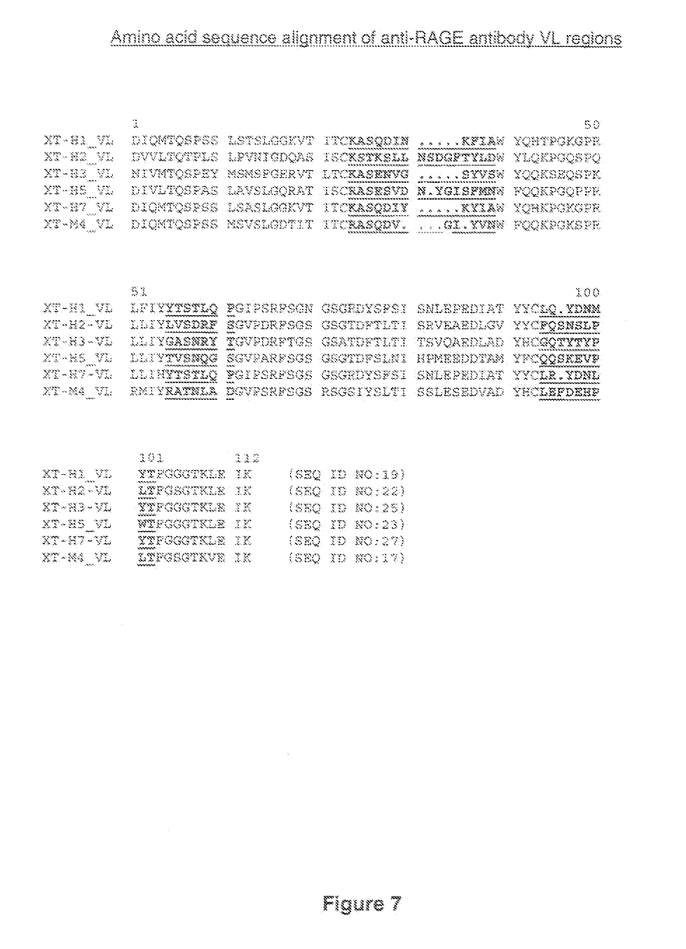

Also included in the invention is an antibody that binds specifically to RAGE and blocks the binding of RAGE to a Rage-binding partner, e.g. a ligands such as HMGB1, AGE, Aβ, SAA, S100, amphoterin, S100P, S100A (including S100A8 and S100A9), S100A4, CRP, β2-integrin, Mac-1, and p150,95, and has CDRs having 4 or more of the following characteristics (position numbering is with respect to amino acid positions as shown for the VH and VL sequences in FIGS. 6 and 7):
1. Amino acid sequence Y-X-M (Y32; X33; M34) in VH CDR1, where X is preferentially W or N;
2. Amino acid sequence I-N-X-S (I51; N52; X53 and S54) in VH CDR2, where X is P or N;
3. Amino acid at position 58 in CDR2 of VH is Threonine;
4. Amino acid at position 60 in CDR2 of VH is Tyrosine;
5. Amino acid at position 103 in CDR3 of VH is Threonine;
6. One or more Tyrosine residues in CDR3 of VH;
7. Positively charged residue (Arg or Lys) at position 24 in CDR1 of VL;
8. Hydrophilic residue (Thr or Ser) at position 26 in CDR1 of VL;
9. Small residue Ser or Ala at the position 25 in CDR1 of VL;
10. Negatively charged residue (Asp or Glu) at position 33 in CDR1 of VL;
11. Aromatic residue (Phe or Tyr or Trp) at position 37 in CDR1 of VL;
12. Hydrophilic residue (Ser or Thr) at position 57 in CDR2 of VL;
13. P-X-T sequence at the end of CDR3 of VL where X could be hydrophobic residue Leu or Trp.

Anti-RAGE antibodies of the invention include antibodies that bind specifically to the V domain of human RAGE and block the binding of RAGE to its ligands, and have CDRs having 5, 6, 7, 8, 9, 10, 11, 12, or all 13 characteristics.

The anti-RAGE antibodies of the invention include an anti-RAGE antibody as described above, or a RAGE-binding fragment which is selected from the group consisting of a chimeric antibody, a humanized antibody, a single chain antibody, a tetrameric antibody, a tetravalent antibody, a multispecific antibody, a domain-specific antibody, a domain-deleted antibody, a fusion protein, an Fab fragment, an Fab′ fragment, an F(ab′)₂ fragment, an Fv fragment, an ScFv fragment, an Fd fragment, a single domain antibody, a dAb fragment, and an Fc fusion protein (i.e., an antigen binding domain fused to an immunoglobulin constant region). These antibodies can be coupled with a cytotoxic agent, a radiotherapeutic agent, or a detectable label.

For example, an ScFv antibody (SEQ ID NO: 63) comprising the VH and VL domains of the rat XT-M4 antibody has been prepared and shown by cell-based ELISA analysis to have binding affinities for RAGE of baboon, mouse, rabbit, and human comparable to those of the chimeric and wild-type XT-M4 antibodies.

Antibodies of the present invention are further intended to include heteroconjugates, bispecific, single-chain, and chimeric and humanized molecules having affinity for one of the subject polypeptides, conferred by at least one CDR region of the antibody.

Antibodies of the invention that specifically bind to RAGE also include variants of any of the antibodies described herein, which may be readily prepared using known molecular biology and cloning techniques. See, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties. For example, a variant antibody of the invention may also comprise a binding domain-immunoglobulin fusion protein that includes a binding domain polypeptide (e.g., scFv) that is fused or otherwise connected to an immunoglobulin hinge or hinge-acting region polypeptide, which in turn is fused or otherwise connected to a region comprising one or more native or engineered constant regions from an immunoglobulin heavy chain, other than CH1, for example, the CH2 and CH3 regions of IgG and IgA, or the CH3 and CH4 regions of IgE (see e.g., U.S. 2005/0136049 by Ledbetter, J. et al., which is incorporated by reference, for a more complete description). The binding domain-immunoglobulin fusion protein can further include a region that includes a native or engineered immunoglobulin heavy chain CH2 constant region polypeptide (or CH3 in the case of a construct derived in whole or in part from IgE) that is fused or otherwise connected to the hinge region polypeptide and a native or engineered immunoglobulin heavy chain CH3 constant region polypeptide (or CH4 in the case of a construct derived in whole or in part from IgE) that is fused or otherwise connected to the CH2 constant region polypeptide (or CH3 in the case of a construct derived in whole or in part from IgE). Typically, such binding domain-immunoglobulin fusion proteins are capable of at least one immunological activity, for example, specific binding to RAGE, inhibition of interaction between RAGE and a RAGE binding partner, induction of antibody dependent cell-mediated cytotoxicity, induction of complement fixation, etc.

Antibodies of the invention may also comprise a label attached thereto and able to be detected, (e.g. the label can be a radioisotope, fluorescent compound, enzyme or enzyme co-factor).

RAGE Polypeptides

The invention also provides isolated RAGE proteins of baboon, cynomologus monkey and rabbit, having the amino acid sequences shown in SEQ ID NOs: 7, 9, 11, or 13, and further includes RAGE proteins having an amino acid sequence that is substantially identical to an amino acid sequences shown in SEQ ID NOs: 7, 9, 11, or 13, in that it is at least 90%, 91%, 92%, 93%, 94%, 95%, 96%, 97%, 98%, 99%, 99.5%, or 99.9% identical in amino acid sequence to any one of SEQ ID NOs: 7, 9, 11, or 13.

Also included in the invention are methods for producing the anti-RAGE antibodies and RAGE-binding fragments thereof of the invention by any means known in the art.

Also Included in the invention is a purified preparation of monoclonal antibody that binds specifically to one or more epitopes of the RAGE amino acid sequence as set forth in any SEQ ID NOs:1, 3, 7, 9, 11, or 13.

### DEFINITIONS

For convenience, certain terms employed in the specification, examples, and appended claims are collected here. Unless defined otherwise, all technical and scientific terms used herein have the same meaning as commonly understood by one of ordinary skill in the art to which this invention belongs.

The articles “a” and “an” are used herein to refer to one or to more than one (i.e., to at least one) of the grammatical object of the article. By way of example, “an element” means one element or more than one element.

The term “or” is used herein to mean, and is used interchangeably with, the term “and/or,” unless context clearly indicates otherwise.

An “isolated” or “purified” polypeptide or protein, e.g., an “isolated antibody,” is purified to a state beyond that in which it exists in nature. For example, the “isolated” or “purified” polypeptide or protein, e.g., an “isolated antibody,” can be substantially free of cellular material or other contaminating proteins from the cell or tissue source from which the protein is derived, or substantially free from chemical precursors or other chemicals when chemically synthesized. The preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation. Proteins or polypeptides referred to herein as “recombinant” are proteins or polypeptides produced by the expression of recombinant nucleic acids.

The term “antibody” is used interchangeably with the term “immunoglobulin” herein, and includes intact antibodies, fragments of antibodies, e.g., Fab, F(ab′)₂ fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced IL 13 binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include Fab, Fab′, F(ab′)₂, Fabc, Fd, dAb, and scFv and/or Fv fragments. The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to RAGE, and neutralizes or inhibits one or more RAGE-associated activities (e.g., inhibits binding of RAGE binding partners (RAGE-BPs) to RAGE).

The antibody includes a molecular structure comprised of four polypeptide chains, two heavy (H) chains and two light (L) chains inter-connected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDRs), interspersed with regions that are more conserved, termed framework regions (FR). Each VH and VL is composed of three CDRs and four FRs, arranged from amino-terminus to carboxy-terminus in the following order: FR1, CDR1, FR2, CDR2, FR3, CDR3, FR4.

It is intended that the term “antibody” encompass any Ig class or any Ig subclass (e.g. the IgG₁, IgG₂, IgG₃, and IgG₄ subclassess of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG₁, IgG₂, IgG₃, and IgG₄) that have been identified in humans and higher mammals. The antibodies can exist in monomeric or polymeric form; for example, IgM antibodies exist in pentameric form, and IgA antibodies exist in monomeric, dimeric or multimeric form.

The term “IgG subclass” refers to the four subclasses of immunoglobulin class IgG—IgG₁, IgG₂, IgG₃, and IgG₄ that have been identified in humans and higher mammals by the γ heavy chains of the immunoglobulins, γ₁-γ₄, respectively.

The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by .beta.-pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

“Specific binding” of an antibody means that the antibody exhibits appreciable affinity for a particular antigen or epitope and, generally, does not exhibit significant crossreactivity. The term “anti-RAGE antibody” as used herein refers to an antibody that binds specifically to a RAGE. The antibody may exhibit no crossreactivity (e.g., does not crossreact with non-RAGE peptides or with remote epitopes on RAGE. “Appreciable” binding includes binding with an affinity of at least 10⁶, 10⁷, 10⁸, 10⁹ M⁻¹, or 10¹⁰ M⁻¹. Antibodies with affinities greater than 10⁷ M⁻¹ or 10⁸ M⁻¹ typically bind with correspondingly greater specificity. Values intermediate of those set forth herein are also intended to be within the scope of the present invention and antibodies of the invention bind to RAGE with a range of affinities, for example, 10⁶ to 10¹⁰ M⁻¹, or 10⁷ to 10¹⁰ M⁻¹, or 10⁸ to 10¹⁰ M⁻¹. An antibody that “does not exhibit significant crossreactivity” is one that will not appreciably bind to an entity other than its target (e.g., a different epitope or a different molecule). For example, an antibody that specifically binds to RAGE will appreciably bind RAGE but will not significantly react with non-RAGE proteins or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The BIACORE™ method relies on the phenomenon of surface plasmon resonance (SPR), which occurs when surface plasmon waves are excited at a metal/liquid interface. Light is directed at, and reflected from, the side of the surface not in contact with sample, and SPR causes a reduction in the reflected light intensity at a specific combination of angle and wavelength. Bimolecular binding events cause changes in the refractive index at the surface layer, which are detected as changes in the SPR signal.

The dissociation constant, Kd, and the association constant, Ka, are quantitative measures of affinity. At equilibrium, free antigen (Ag) and free antibody (Ab) are in equilibrium with antigen-antibody complex (Ag−Ab), and the rate constants, ka and kd, quantitate the rates of the individual reactions:
ka kd
   Ab+Ag→Ab−Ag and Ab−Ag→Ab+Ag

At equilibrium, ka [Ab][Ag]=kd [Ag−Ab]. The dissociation constant, Kd, is given by: Kd=kd/ka=[Ag][Ab]/[Ag−Ab]. Kd has units of concentration, most typically M, mM, pM, nM, pM, etc. When comparing antibody affinities expressed as Kd, having greater affinity for RAGE is indicated by a lower value. The association constant, Ka, is given by: Ka=ka/kd=[Ag−Ab]/[Ag][Ab]. Ka has units of inverse concentration, most typically M⁻¹, mM⁻¹, μM⁻¹, nM⁻¹, pM⁻¹, etc. As used herein, the term “avidity” refers to the strength of the antigen-antibody bond after formation of reversible complexes. Anti-RAGE antibodies may be characterized in terms of the Kd for their binding to a RAGE protein, as binding “with a dissociation constant (Kd) in the range of from about (lower Kd value) to about (upper Kd value).” In this context, the term “about” is intended to mean the indicated Kd value ±20%; i.e., Kd of about 1=Kd in the range of from 0.8 to 1.2.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to RAGE. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)₂, Fabc, Fd, dAb, Fv, single chains, single-chain antibodies, e.g., scFv, and single domain antibodies (Muyldermans et al., 2001, 26:230-5), and an isolated complementarity determining region (CDR). Fab fragment is a monovalent fragment consisting of the VL, VH, CL and CH1 domains. F(ab′)₂ fragment is a bivalent fragment comprising two Fab fragments linked by a disulfide bridge at the hinge region. Fd fragment consists of the VH and CH1 domains, and Fv fragment consists of the VL and VH domains of a single arm of an antibody. A dAb fragment consists of a VH domain (Ward et al., (1989) Nature 341:544-546). While the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv) (Bird et al., 1988, Science 242:423-426). Such single chain antibodies are also intended to be encompassed within the term “binding portion” of an antibody. Other forms of single chain antibodies, such as diabodies are also encompassed. Diabodies are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see e.g., Holliger, et al., 1993, Proc. Natl. Acad. Sci. USA 90:6444-6448). An antibody or binding portion thereof also may be part of a larger immunoadhesion molecules formed by covalent or non-covalent association of the antibody or antibody portion with one or more other proteins or peptides. Examples of such immunoadhesion molecules include use of the streptavidin core region to make a tetrameric scFv molecule (Kipriyanov, S. M., et al. (1995) Human Antibodies and Hybridomas 6:93-101) and use of a cysteine residue, a marker peptide and a C-terminal polyhistidine tag to make bivalent and biotinylated scFv molecules (Kipriyanov, S. M., et al. (1994) Mol. Immunol. 31:1047-1058). Binding fragments such as Fab and F(ab′)₂ fragments, can be prepared from whole antibodies using conventional techniques, such as papain or pepsin digestion, respectively, of whole antibodies. Moreover, antibodies, antibody portions and immunoadhesion molecules can be obtained using standard recombinant DNA techniques, as described herein and as known in the art. Other than “bispecific” or “bifunctional” antibodies, an antibody is understood to have each of its binding sites identical. A “bispecific” or “bifunctional antibody” is an artificial hybrid antibody having two different heavy/light chain pairs and two different binding sites. A bispecific antibody can also include two antigen binding regions with an intervening constant region. Bispecific antibodies can be produced by a variety of methods including fusion of hybridomas or linking of Fab′ fragments. See, e.g., Songsivilai et al., Clin. Exp. Immunol. 79:315-321, 1990; Kostelny et al., 1992, J. Immunol. 148, 1547-1553.

The term “backmutation” refers to a process in which some or all of the somatically mutated amino acids of a human antibody are replaced with the corresponding germline residues from a homologous germline antibody sequence. The heavy and light chain sequences of the human antibody of the invention are aligned separately with the germline sequences in the VBASE database to identify the sequences with the highest homology. Differences in the human antibody of the invention are returned to the germline sequence by mutating defined nucleotide positions encoding such different amino acid. The role of each amino acid thus identified as candidate for backmutation should be investigated for a direct or indirect role in antigen binding and any amino acid found after mutation to affect any desirable characteristic of the human antibody should not be included in the final human antibody; as an example, activity enhancing amino acids identified by the selective mutagenesis approach will not be subject to backmutation. To minimize the number of amino acids subject to backmutation those amino acid positions found to be different from the closest germline sequence but identical to the corresponding amino acid in a second germline sequence can remain, provided that the second germline sequence is identical and colinear to the sequence of the human antibody of the invention for at least 10, preferably 12 amino acids, on both sides of the amino acid in question. Backmutation may occur at any stage of antibody optimization; preferably, backmutation occurs directly before or after the selective mutagenesis approach. More preferably, backmutation occurs directly before the selective mutagenesis approach.

Intact antibodies, also known as immunoglobulins, are typically tetrameric glycosylated proteins composed of two light (L) chains of approximately 25 kDa each and two heavy (H) chains of approximately 50 kDa each. Two types of light chain, termed lambda and kappa, are found in antibodies. Depending on the amino acid sequence of the constant domain of heavy chains, immunoglobulins can be assigned to five major classes: A, D, E, G, and M, and several of these may be further divided into subclasses (isotypes), e.g., IgG1, IgG2, IgG3, IgG4, IgA1, and IgA2. Each light chain is composed of an N terminal variable (V) domain (VL) and a constant (C) domain (CL). Each heavy chain is composed of an N terminal V domain (VH), three or four C domains (CHs), and a hinge region. The CH domain most proximal to VH is designated as CH1. The VH and VL domains consist of four regions of relatively conserved sequences called framework regions (FR1, FR2, FR3, and FR4), which form a scaffold for three regions of hypervariable sequences (complementarity determining regions, CDRs). The CDRs contain most of the residues responsible for specific interactions of the antibody with the antigen. CDRs are referred to as CDR1, CDR2, and CDR3. Accordingly, CDR constituents on the heavy chain are referred to as H1 , H2, and H3, while CDR constituents on the light chain are referred to as L1 , L2, and L3. CDR3 is the greatest source of molecular diversity within the antibody-binding site. H3, for example, can be as short as two amino acid residues or greater than 26 amino acids. The subunit structures and three-dimensional configurations of different classes of immunoglobulins are well known in the art. For a review of the antibody structure, see Antibodies: A Laboratory Manual, Cold Spring Harbor Laboratory, eds. Harlow et al., 1988. One of skill in the art will recognize that each subunit structure, e.g., a CH, VH, CL, VL, CDR, FR structure, comprises active fragments, e.g., the portion of the VH, VL, or CDR subunit that binds to the antigen, i.e., the binding fragment, or, e.g., the portion of the CH subunit that binds to and/or activates, e.g., an Fc receptor and/or complement.

Antibody diversity is created by the use of multiple germline genes encoding variable regions and a variety of somatic events. The somatic events include recombination of variable gene segments with diversity (D) and joining (J) gene segments to make a complete VH region, and the recombination of variable and joining gene segments to make a complete VL region. The recombination process itself is imprecise, resulting in the loss or addition of amino acids at the V(D)J junctions. These mechanisms of diversity occur in the developing B-cell prior to antigen exposure. After antigenic stimulation, the expressed antibody genes in B-cells undergo somatic mutation. Based on the estimated number of germline gene segments, the random recombination of these segments, and random VH-VL pairing, up to 1.6×107 different antibodies could be produced (Fundamental Immunology, 3rd ed. (1993), ed. Paul, Raven Press, New York, N.Y.). When other processes that contribute to antibody diversity (such as somatic mutation) are taken into account, it is thought that upwards of 1×1010 different antibodies could be generated (Immunoglobulin Genes, 2nd ed. (1995), eds. Jonio et al., Academic Press, San Diego, Calif.). Because of the many processes involved in generating antibody diversity, it is unlikely that independently derived monoclonal antibodies with the same antigen specificity will have identical amino acid sequences.

The term “dimerizing polypeptide” or “dimerizing domain” includes any polypeptide that forms a diner (or higher order complex, such as a trimer, tetramer, etc.) with another polypeptide. Optionally, the dimerizing polypeptide associates with other, identical dimerizing polypeptides, thereby forming homomultimers. An IgG Fc element is an example of a dimerizing domain that tends to form homomultimers. Optionally, the dimerizing polypeptide associates with other different dimerizing polypeptides, thereby forming heteromultimers. The Jun leucine zipper domain forms a dimer with the Fos leucine zipper domain, and is therefore an example of a dimerizing domain that tends to form heteromultimers. Dimerizing domains may form 25 both hetero- and homomultimers.

The term “human antibody” includes antibodies having variable and constant regions corresponding to human germline immunoglobulin sequences as described by Kabat et al. (See Kabat, et al. (1991) Sequences of proteins of Immunological Interest, Fifth Edition, U.S. Department of Health and Human Services, NIH Publication No. 91-3242). The human antibodies of the invention may include amino acid residues not encoded by human germline immunoglobulin sequences (e.g., mutations introduced by random or site-specific mutagenesis in vitro or by somatic mutation in vivo), for example in the CDRs and in particular CDR3. The mutations preferably are introduced using the “selective mutagenesis approach” described herein. The human antibody can have at least one position replaced with an amino acid residue, e.g., an activity enhancing amino acid residue, which is not encoded by the human germline immunoglobulin sequence. The human antibody can have up to twenty positions replaced with amino acid residues that are not part of the human germline immunoglobulin sequence. Further, up to ten, up to five, up to three or up to two positions are replaced. These replacements may fall within the CDR regions as described in detail below. However, the term “human antibody”, as used herein, is not intended to include antibodies in which CDR sequences derived from the germline of another mammalian species, such as a mouse, have been grafted onto human framework sequences.

The phrase “recombinant human antibody” includes human antibodies that are prepared, expressed, created or isolated by recombinant means, such as antibodies expressed using a recombinant expression vector transfected into a host cell (described further in Section II, below), antibodies isolated from a recombinant, combinatorial human antibody library (described further in Section III, below), antibodies isolated from an animal (e.g., a mouse) that is transgenic for human immunoglobulin genes (see e.g., Taylor, L. D., et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences (See Kabat, E. A., et al. (1991) Sequences of Proteins of Immunological Interest, Fifth Edition, U.S. Department of Health and Human Services, NIH Publication No. 91-3242). However, such recombinant human antibodies may be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. In certain embodiments, however, such recombinant antibodies may be the result of selective mutagenesis approach or backmutation or both.

An “isolated antibody” includes an antibody that is substantially free of other antibodies having different antigenic specificities (e.g., an isolated antibody that specifically binds RAGE is substantially free of antibodies that specifically bind RAGE other than hRAGE). An isolated antibody that specifically binds RAGE may bind RAGE molecules from other species. Moreover, an isolated antibody may be substantially free of other cellular material and/or chemicals.

A “neutralizing antibody” (or an “antibody that neutralized RAGE activity”) includes an antibody whose binding to hRAGE results in modulation of the biological activity of hRAGE. This modulation of the biological activity of hRAGE can be assessed by measuring one or more indicators of hRAGE biological activity, such as inhibition of receptor binding in a human RAGE receptor binding assay (see, e.g., Examples 6 and 7). These indicators of hRAGE biological activity can be assessed by one or more of several standard in vitro or in vivo assays known in the art (see, e.g., Examples 6 and 7).

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

The term “activity” includes activities such as the binding specificity/affinity of an antibody for an antigen, for example, an anti-hRAGE antibody that binds to RAGE and/or the neutralizing potency of an antibody, for example, an anti-hRAGE antibody whose binding to hRAGE inhibits the biological activity of RAGE, e.g., inhibition of receptor binding in a human RAGE receptor binding assay.

An “expression construct” is any recombinant nucleic acid that includes an expressible nucleic acid and regulatory elements sufficient to mediate expression of the expressible nucleic acid protein or polypeptide in a suitable host cell.

The terms “fusion protein” and “chimeric protein” are interchangeable and refer to a protein or polypeptide that has an amino acid sequence having portions corresponding to amino acid sequences from two or more proteins. The sequences from two or more proteins may be full or partial (i.e., fragments) of the proteins. Fusion proteins may also have linking regions of amino acids between the portions corresponding to those of the proteins. Such fusion proteins may be prepared by recombinant methods, wherein the corresponding nucleic acids are joined through treatment with nucleases and ligases and incorporated into an expression vector. Preparation of fusion proteins is generally understood by those having ordinary skill in the art.

The term “nucleic acid” refers to polynucleotides such as deoxyribonucleic acid (DNA), and, where appropriate, ribonucleic acid (RNA). The term should also be understood to include, as equivalents, analogs of either RNA or DNA made from nucleotide analogs, and, as applicable to the embodiment being described, single (sense or antisense) and double-stranded polynucleotides.

The term “percent identical” or “percent identity” refers to sequence identity between two amino acid sequences or between two nucleotide sequences. Percent identity can be determined by comparing a position in each sequence that may be aligned for purposes of comparison. Expression as a percentage of identity refers to a function of the number of identical amino acids or nucleic acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g. default settings. ENTREZ is available through the National Center for Biotechnology Information, National Library of Medicine, National Institutes of Health, Bethesda, Md. The percent identity of two sequences may be determined by the GCG program with a gap weight of 1, e.g. each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. Preferably, an alignment program that permits gaps in the sequence is utilized to align the sequences. The Smith-Waterman is one type of algorithm that permits gaps in sequence alignments. See Meth. Mol. Viols. 70: 173-187 (1997). Also, the GAP I program using the Needlenan and Wunsch alignment method can be utilized to align sequences. An alternative search strategy uses MPSRCH software, which runs on a MASPAR computer. MPSRCH uses a Smith-Waterman algorithm to score sequences 5 on a massively parallel computer. This approach improves the ability to pick up distantly related matches, and is especially tolerant of small gaps and nucleotide sequence errors. Nucleic acid-encoded amino acid sequences can be used to search both protein: and DNA databases.

The terms “polypeptide” and “protein” are used interchangeably herein.

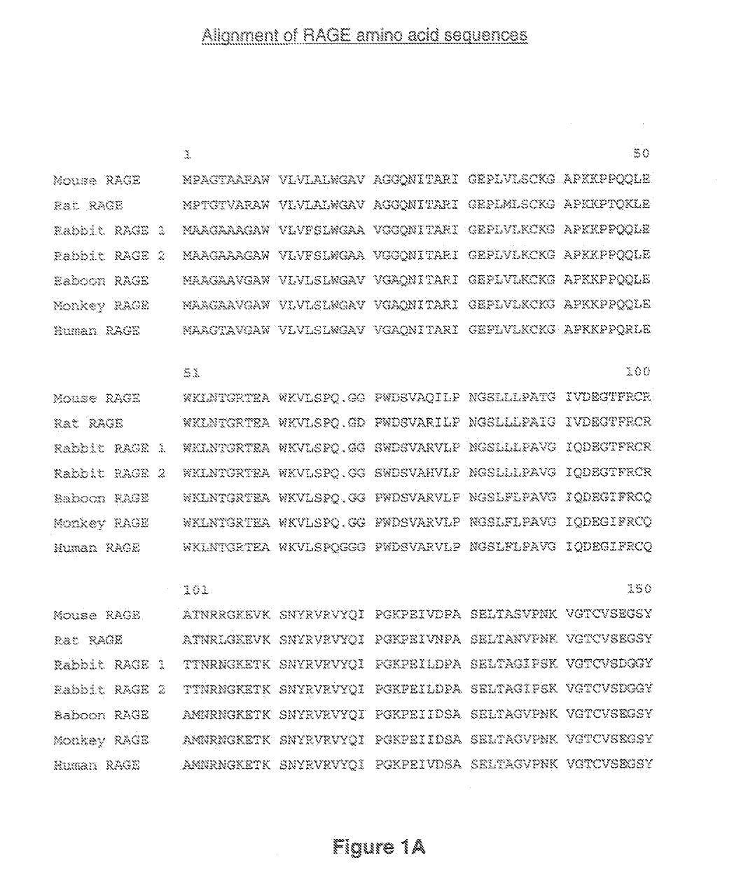

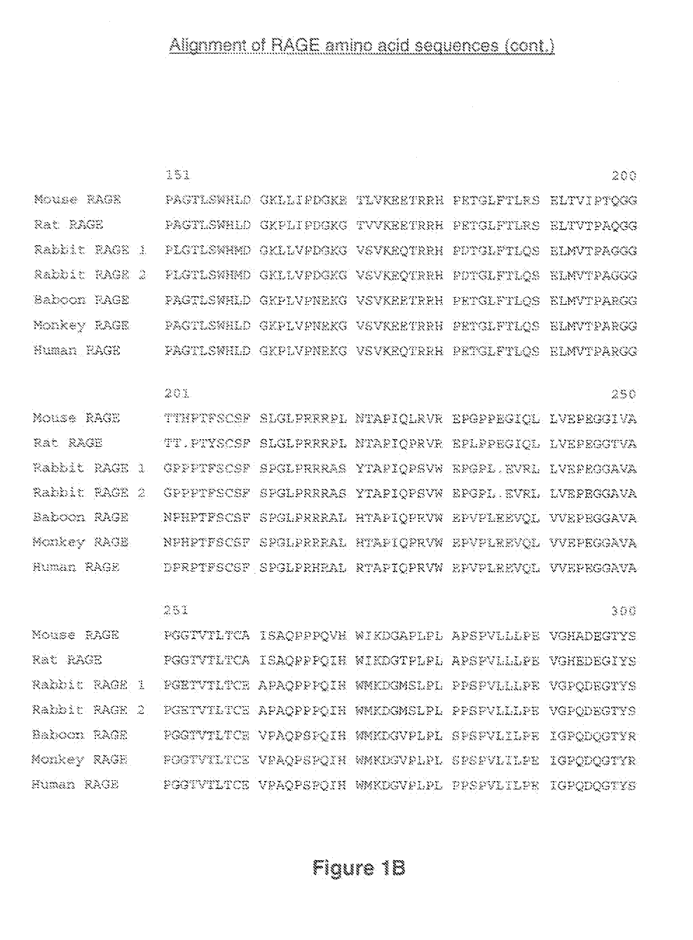

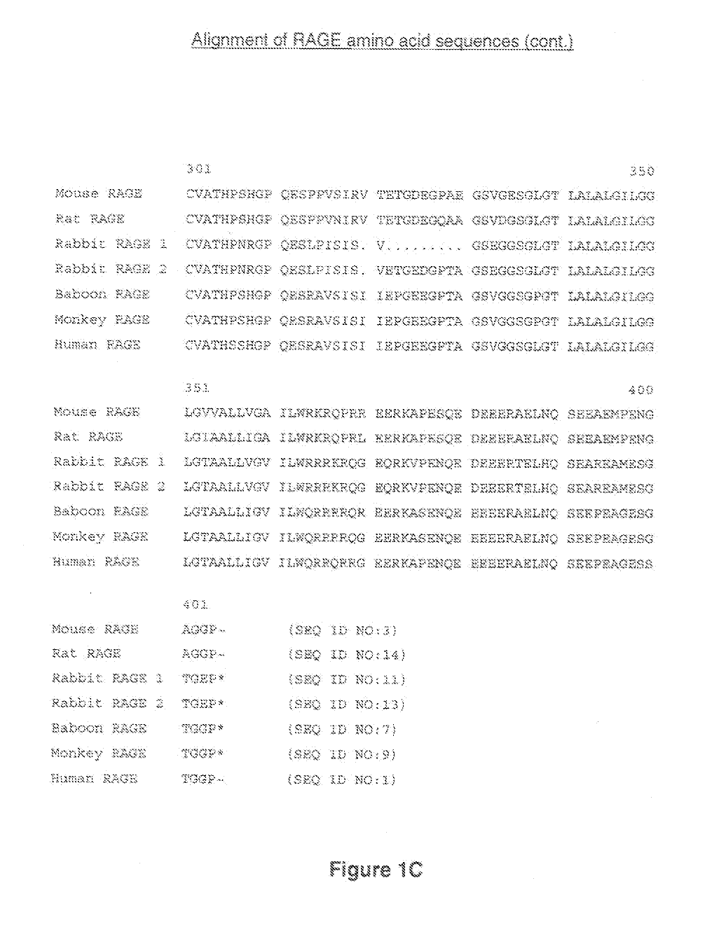

A “RAGE” protein is a “Receptor for Advanded Glycation End Products,” as known in the art. Representative RAGE proteins are set forth in FIGS. 1A-1C. RAGE proteins include soluble RAGE (sRAGE) and endogenous secretory RAGE (esRAGE). Endogenous secretory RAGE is a RAGE splice variant that is released outside of the cells, where it is capable of binding AGE ligands and neutralizing AGE actions. See e.g., Koyama et al., ATVE, 2005; 25:2587-2593. Inverse association has been observed between human plasma esRAGE and several components of metabolic syndrome (BMI, insulin resistance, BP, hypertriglyceridemia and IGT). Plasma esRAGE levels have also been inversely associated with carotid and femoral atherosclerosis (quantitated by ultrasound) in subjects with or without diabetes. Moreover, plasma esRAGE levels are significantly lower in nondiabetic patients with angiographically proved coronary artery disease than age-matched healthy control.

A “Receptor for Advanced Glycation End Products Ligand Binding Element” or “RAGE-LBE” (also referred to herein as “RAGE-Fc” and “RAGE-strep”) includes any extracellular portion of a transmembrane RAGE polypeptide and fragments thereof that retain the ability to bind a RAGE ligand. This term also encompasses polypeptides having at least 85% identity, preferably at least 90% identity or more preferably at least 95% identity with a RAGE polypeptide, for example, the human or murine polypeptide to which a RAGE ligand or RAGE-BP will bind.

A “Receptor for Advanced Glycation End Products Binding Partner” or “RAGE-BP” includes any substance (e.g., polypeptide, small molecule, carbohydrate structure, etc.) that binds in a physiological setting to an extracellular portion of a RAGE protein (a receptor polypeptide such as, e.g., RAGE or RAGE-LBE).

“RAGE-related disorders” or “RAGE-associated disorders” include any disorder in which an affected cell or tissue exhibits an increase or decrease in the expression and/or activity of RAGE or one or more RAGE ligands. RAGE-related disorders also include any disorder that is treatable (i.e., one or more symptom may be eliminated or ameliorated) by a decrease in RAGE function (including, for example, administration of an agent that disrupts RAGE:RAGE-BP interactions).

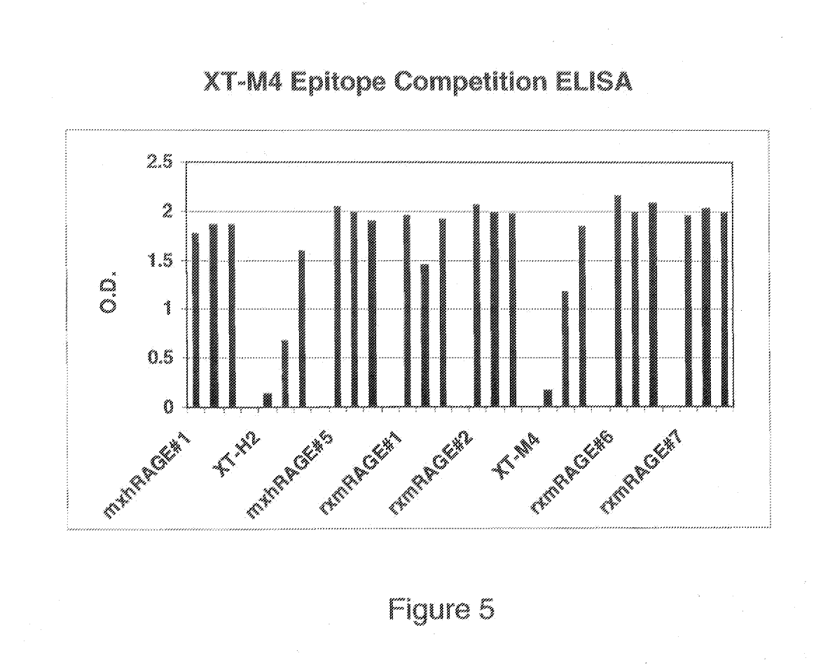

“V-domain of RAGE” refers to the immunoglobulin-like variable domain as shown in FIG. 5 of Neeper, et al, “Cloning and expression of RAGE: a cell surface receptor for advanced glycosylation end products of proteins,” J. Biol. Chem. 267:14998-15004 (1992), the contents of which are hereby incorporated by reference. The V-domain includes amino acids from position 1 to position 120 as shown in SEQ ID NO:1 and SEQ ID NO:3.

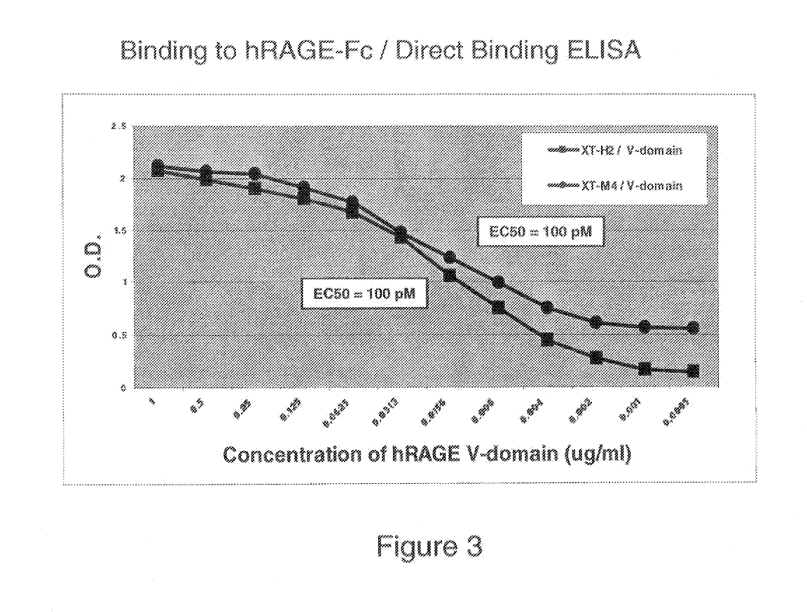

The human cDNA of RAGE is 1406 base pairs and encodes a mature protein of 404 amino acids. See FIG. 3 of Neeper et al. 1992.

The term “recombinant nucleic acid” includes any nucleic acid comprising at least two sequences that are not present together in nature. A recombinant nucleic acid may be generated in vitro, for example by using the methods of molecular biology, or in vivo, for example by insertion of a nucleic acid at a novel chromosomal location by homologous or non-homologous recombination.

The term “treating” with regard to a subject, refers to improving at least one symptom of the subject's disease or disorder. Treating can be curing the disease or condition or improving it.

The term “vector” refers to a nucleic acid molecule capable of transporting another nucleic acid to which it has been linked. One type of vector is an episome, i.e., a nucleic acid capable of extra-chromosomal replication. Another type of vector is an integrative vector that is designed to recombine with the genetic material of a host cell. Vectors may be both autonomously replicating and integrative, and the properties of a vector may differ depending on the cellular context (i.e., a vector may be autonomously replicating in one host cell type and purely integrative in another host cell type). Vectors capable of directing the expression of expressible nucleic acids to which they are operatively linked are referred to herein as “expression vectors.”

“Specifically immunoreactive” refers to the preferential binding of compounds [an antibody] to a particular peptide sequence, when an antibody interacts with a specific peptide sequence.

The phrase “effective amount” as used herein means that amount of one or more agent, material, or composition comprising one or more agents of the present invention that is effective for producing some desired effect in an animal. It is recognized that when an agent is being used to achieve a therapeutic effect, the actual dose which comprises the “effective amount” will vary depending on a number of conditions including the particular condition being treated, the severity of the disease, the size and health of the patient, the route of administration, etc. A skilled medical practitioner can readily determine the appropriate dose using methods well known in the medical arts.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable carrier” as used herein means a pharmaceutically acceptable material, composition or vehicle, such as a liquid or solid filler, diluent, excipient, solvent or encapsulating material, involved in carrying or transporting the subject agents from one organ, or portion of the body, to another organ, or portion of the body. Each carrier must be “acceptable” in the sense of being compatible with the other ingredients of the formulation. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline, (18) Ringer's solution, (19) ethyl alcohol; (20) phosphate buffer solutions; and (21) other non-toxic compatible substances employed in pharmaceutical formulations.

Preparation of Monoclonal Antibodies

A mammal, such as a mouse, a rat, a hamster or rabbit can be immunized with the full length protein or fragments thereof, or the cDNA encoding the full length protein or a fragment thereof an immunogenic form of the peptide. Techniques for conferring immunogenicity on a protein or peptide include conjugation to carriers or other techniques well known in the art. An immunogenic portion of a polypeptide can be administered in the presence of adjuvant. The progress of immunization can be monitored by detection of antibody titers in plasma or serum. Standard ELISA or other immunoassays can be used with the immunogen as antigen to assess the levels of antibodies.

Following immunization of an animal with an antigenic preparation of the subject polypeptides, antisera can be obtained and, if desired, polyclonal antibodies isolated from the serum. To produce monoclonal antibodies, antibody-producing cells (lymphocytes) can be harvested from an immunized animal and fused by standard somatic cell fusion procedures with immortalizing cells such as myeloma cells to yield hybridoma cells. Such techniques are well known in the art, and include, for example, the hybridoma technique (originally developed by Kohler and Milstein, (1975) Nature, 256: 495-497), the human B cell hybridoma technique (Kozbar et al. (1983) Immunology Today, 4: 72), and the EBV-hybridoma technique to produce human monoclonal antibodies (Cole et al., (1985) Monoclonal Antibodies and Cancer Therapy, Alan R. Liss, Inc. pp. 77-96). Hybridoma cells can be screened immunochemically for production of antibodies specifically reactive with an epitope of the RAGE polypeptide and monoclonal antibodies isolated from a culture comprising such hybridoma cells.

Humanization

Chimeric antibodies comprise sequences from at least two different species. As one example, recombinant cloning techniques may be used to include variable regions, which contain the antigen-binding sites, from a non-human antibody (i.e., an antibody prepared in a non-human species immunized with the antigen) and constant regions derived from a human immunoglobulin.

Humanized antibodies are a type of chimeric antibody wherein variable region residues responsible for antigen binding (i.e., residues of a complementarity determining region, abbreviated complementarity determining region, or any other residues that participate in antigen binding) are derived from a non-human species, while the remaining variable region residues (i.e., residues of the framework regions) and constant regions are derived, at least in part, from human antibody sequences. A subset of framework region residues and constant region residues of a humanized antibody may be derived from non-human sources. Variable regions of a humanized antibody are also described as humanized (i.e., a humanized light or heavy chain variable region). The non-human species is typically that used for immunization with antigen, such as mouse, rat, rabbit, non-human primate, or other non-human mammalian species. Humanized antibodies are typically less immunogenic than traditional chimeric antibodies and show improved stability following administration to humans. See e.g., Benincosa et al. (2000) J. Pharmacol. Exp. Ther. 292:810-6; Kalofonos et al. (1994) Eur. J. Cancer 30A:1842-50; Subramanian et al. (1998) Pediatr. Infect. Dis. J. 17:110-5.

Complementarity determining regions (CDRs) are residues of antibody variable regions that participate in antigen binding. Several numbering systems for identifying CDRs are in common use. The Kabat definition is based on sequence variability, and the Chothia definition is based on the location of the structural loop regions. The AbM definition is a compromise between the Kabat and Chothia approaches. The CDRs of the light chain variable region are bounded by the residues at positions 24 and 34 (CDR1-L), 50 and 56 (CDR2-L), and 89 and 97 (CDR3-L) according to the Kabat, Chothia, or AbM algorithm. According to the Kabat definition, the CDRs of the heavy chain variable region are bounded by the residues at positions 31 and 35B (CDR1-H), 50 and 65 (CDR2-H), and 95 and 102 (CDR3-H) (numbering according to Kabat). According to the Chothia definition, the CDRs of the heavy chain variable region are bounded by the residues at positions 26 and 32 (CDR1-H), 52 and 56 (CDR2-H), and 95 and 102 (CDR3-H) (numbering according to Chothia). According to the AbM definition, the CDRs of the heavy chain variable region are bounded by the residues at positions 26 and 35B (CDR1-H), 50 and 58 (CDR2-H), and 95 and 102 (CDR3-H) (numbering according to Kabat). See Martin et al. (1989) Proc. Natl. Acad. Sci. USA 86: 9268-9272; Martin et al. (1991) Methods Enzymol. 203: 121-153; Pedersen et al. (1992) Immunomethods 1: 126; and Rees et al. (1996) In Sternberg M. J. E. (ed.), Protein Structure Prediction, Oxford University Press, Oxford, pp. 141-172.

As used herein, the term “CDR” refer to CDRs as defined either by Kabat or by Chothia; moreover, a humanized antibody variable of the invention may be constructed to comprise one or more CDRs as defined by Kabat, and to also comprise one or more CDRs as defined by Chothia.

Specificity determining regions (SDRs) are residues within CDRs that directly interact with antigen. The SDRs correspond to hypervariable residues. See (Padlan et al. (1995) FASEB J. 9: 133-139).

Framework residues are those residues of antibody variable regions other than hypervariable or CDR residues. Framework residues may be derived from a naturally occurring human antibody, such as a human framework that is substantially similar to a framework region of the an anti-RAGE antibody of the invention. Artificial framework sequences that represent a consensus among individual sequences may also be used. When selecting a framework region for humanization, sequences that are widely represented in humans may be preferred over less populous sequences. Additional mutations of the human framework acceptor sequences may be made to restore murine residues believed to be involved in antigen contacts and/or residues involved in the structural integrity of the antigen-binding site, or to improve antibody expression. A peptide structure prediction may be used to analyze the humanized variable heavy and light region sequences to identify and avoid post-translational protein modification sites introduced by the humanization design.

Humanized antibodies may be prepared using any one of a variety of methods including veneering, grafting of complementarity determining regions (CDRs), grafting of abbreviated CDRs, grafting of specificity determining regions (SDRs), and Frankenstein assembly, as described below. Humanized antibodies also include superhumanized antibodies, in which one or more changes have been introduced in the CDRs. For example, human residues may be substituted for non-human residues in the CDRs. These general approaches may be combined with standard mutagenesis and synthesis techniques to produce an anti-RAGE antibody of any desired sequence.

Veneering is based on the concept of reducing potentially immunogenic amino acid sequences in a rodent or other non-human antibody by resurfacing the solvent accessible exterior of the antibody with human amino acid sequences. Thus, veneered antibodies appear less foreign to human cells than the unmodified non-human antibody. See Padlan (1991) Mol. Immunol. 28:489-98. A non-human antibody is veneered by identifying exposed exterior framework region residues in the non-human antibody, which are different from those at the same positions in framework regions of a human antibody, and replacement of the identified residues with amino acids that typically occupy these same positions in human antibodies.

Grafting of CDRs is performed by replacing one or more CDRs of an acceptor antibody (e.g., a human antibody or other antibody comprising desired framework residues) with CDRs of a donor antibody (e.g., a non-human antibody). Acceptor antibodies may be selected based on similarity of framework residues between a candidate acceptor antibody and a donor antibody. For example, according to the Frankenstein approach, human framework regions are identified as having substantial sequence homology to each framework region of the relevant non-human antibody, and CDRs of the non-human antibody are grafted onto the composite of the different human framework regions. A related method also useful for preparation of antibodies of the invention is described in U.S. Patent Application Publication No. 2003/0040606.

Grafting of abbreviated CDRs is a related approach. Abbreviated CDRs include the specificity-determining residues and adjacent amino acids, including those at positions 27d-34, 50-55 and 89-96 in the light chain, and at positions 31-35b, 50-58, and 95-101 in the heavy chain (numbering convention of (Kabat et al. (1987)). See (Padlan et al. (1995) FASEB J. 9: 133-9). Grafting of specificity-determining residues (SDRs) is premised on the understanding that the binding specificity and affinity of an antibody combining site is determined by the most highly variable residues within each of the complementarity determining regions (CDRs). Analysis of the three-dimensional structures of antibody-antigen complexes, combined with analysis of the available amino acid sequence data may be used to model sequence variability based on structural dissimilarity of amino acid residues that occur at each position within the CDR. SDRs are identified as minimally immunogenic polypeptide sequences consisting of contact residues. See Padlan et al. (1995) FASEB J. 9: 133-139.

Acceptor frameworks for grafting of CDRs or abbreviated CDRs may be further modified to introduce desired residues. For example, acceptor frameworks may comprise a heavy chain variable region of a human sub-group I consensus sequence, optionally with non-human donor residues at one or more of positions 1, 28, 48, 67, 69, 71, and 93. As another example, a human acceptor framework may comprise a light chain variable region of a human sub-group I consensus sequence, optionally with non-human donor residues at one or more of positions 2, 3, 4, 37, 38, 45 and 60. Following grafting, additional changes may be made in the donor and/or acceptor sequences to optimize antibody binding and functionality. See e.g., PCT International Publication No. WO 91/09967.

Human frameworks of a heavy chain variable region that may be used to prepare humanized anti-RAGE antibodies include framework residues of DP-75, DP54, DP-54 FW VH 3 JH4, DP-54 VH3 3-07, DP-8(VH1-2), DP-25, VI-2b and VI-3 (VH1-03), DP-15 and V1-8 (VH1-08), DP-14 and V1-18 (VH1-18), DP-5 and V1-24P (VH1-24), DP-4 (VH1-45), DP-7 (VH1-46), DP-10, DA-6 and YAC-7 (VH1-69), DP-88 (VH1-e), DP-3, and DA-8 (VH1-f).

Human frameworks of a light chain variable region that may be used to prepare humanized anti-RAGE antibodies include framework residues of human germ line clone DPK24, DPK-12, DPK-9 Vk1, DPK-9 Jk4, DPK9 Vk1 02, and germ line clone subgroups VκIII and VκI. The following mutations of a DPK24 germ line may increase antibody expression: F10S, T45K, I63S, Y67S, F73L, and T77S.

Representative humanized anti-RAGE antibodies of the invention include antibodies having one or more CDRs of a variable region amino acid sequence selected from SEQ ID NOs:16-27. For example, humanized anti-RAGE antibodies may comprise two or more CDRs selected from CDRs of a heavy chain variable region of any one of SEQ ID NOs:16, 18, 21, 24, 20, and 26, or a light chain variable region of any one of SEQ ID NOs:17, 19, 22, 25, 23, and 27. Humanized anti-RAGE antibodies may also comprise a heavy chain comprising a variable region having two or three CDRs of any one of SEQ ID NOs:16, 18, 21, 24, 20, and 26, and a light chain comprising a variable region having two or three CDRs of any one of SEQ ID NOs: 17, 19, 22, 25, 23, and 27.

Humanized anti-RAGE antibodies of the invention may be constructed wherein the variable region of a first chain (i.e., the light chain variable region or the heavy chain variable region) is humanized, and wherein the variable region of the second chain is not humanized (i.e., a variable region of an antibody produced in a non-human species). These antibodies are a type of humanized antibody referred to as semi-humanized antibodies.

The constant regions of chimeric and humanized anti-RAGE antibodies may be derived from constant regions of any one of IgA, IgD, IgE, IgG, IgM, and any isotypes thereof (e.g., IgG1, IgG2, IgG3, or IgG4 isotypes of IgG). The amino acid sequences of many antibody constant regions are known. The choice of a human isotype and modification of particular amino acids in the isotype may enhance or eliminate activation of host defense mechanisms and alter antibody biodistribution. See (Reff et al. (2002) Cancer Control 9: 152-66). For cloning of sequences encoding immunoglobulin constant regions, intronic sequences may be deleted.

Chimeric and humanized anti-RAGE antibodies may be constructed using standard techniques known in the art. For example, variable regions may be prepared by annealing together overlapping oligonucleotides encoding the variable regions and ligating them into an expression vector containing a human antibody constant region. See e.g., Harlow & Lane (1988) Antibodies: A Laboratory Manual, Cold Spring Harbor Laboratory Press, Cold Spring Harbor, N.Y. and U.S. Pat. Nos. 4,196,265; 4,946,778; 5,091,513; 5,132,405; 5,260,203; 5,677,427; 5,892,019; 5,985,279; 6,054,561. Tetravalent antibodies (H₄L₄) comprising two intact tetrameric antibodies, including homodimers and heterodimers, may be prepared, for example, as described in PCT International Publication No. WO 02/096948. Antibody dimers may also be prepared via introduction of cysteine residue(s) in the antibody constant region, which promote interchain disulfide bond formation, by use of heterobifunctional cross-linkers (Wolff et al. (1993) Cancer Res. 53: 2560-5), or by recombinant production to include a dual constant region (Stevenson et al. (1989) Anticancer Drug Des. 3: 219-30). Antigen-binding fragments of antibodies of the invention may be prepared, for example, by expression of truncated antibody sequences, or by post-translation digestion of full-length antibodies.

Variants of anti-RAGE antibodies of the invention may be readily prepared to include various changes, substitutions, insertions, and deletions. For example, antibody sequences may be optimized for codon usage in the cell type used for antibody expression. To increase the serum half life of the antibody, a salvage receptor binding epitope may be incorporated, if not present already, into the antibody heavy chain sequence. See U.S. Pat. No. 5,739,277. Additional modifications to enhance antibody stability include modification of IgG4 to replace the serine at residue 241 with proline. See Angal et al. (1993) Mol. Immunol. 30: 105-108. Other useful changes include substitutions as required to optimize efficiency in conjugating the antibody with a drug. For example, an antibody may be modified at its carboxyl terminus to include amino acids for drug attachment, for example one or more cysteine residues may be added. The constant regions may be modified to introduce sites for binding of carbohydrates or other moieties.

Additional antibody variants include glycosylation isoforms that result in improved functional properties. For example, modification of Fc glycosylation can result in altered effector functions, e.g., increased binding to Fc gamma receptors and improved ADCC and/or could decreased C1q binding and CDC (e.g., changing of Fc oligosaccharides from complex form to high-mannose or hybrid type may decrease C1q binding and CDC (see, e.g., Kanda et al., Glycobiology, 2007:17:104-118)). Modification can be done by bioengineering bacteria, yeast, plant cells, insect cells, and mammalian cells; it can also be done by manipulating protein or natural product glycosylation pathways in genetically engineered organisms. Glycosylation can also be altered by exploiting the liberality with which sugar-attaching enzymes (glycosyltransferases) tolerate a wide range of different substrates. Finally, one of skill in the art can glycosylate proteins and natural products through a variety of chemical approaches: with small molecules, enzymes, protein ligation, metabolic bioengineering, or total synthesis. Examples of suitable small molecule inhibitors of N-glycan processing include, Castanospermine (CS), Kifunensine (KF), Deoxymannojirimycin (DMJ), Swainsonine (Sw), Monensin (Mn).

Variants of anti-RAGE antibodies of the invention may be produced using standard recombinant techniques, including site-directed mutagenesis, or recombination cloning. A diversified repertoire of anti-RAGE antibodies may be prepared via gene arrangement and gene conversion methods in transgenic non-human animals (U.S. Patent Publication No. 2003/0017534), which are then tested for relevant activities using functional assays. In particular embodiments of the invention, variants are obtained using an affinity maturation protocol for mutating CDRs (Yang et al. (1995) J. Mol. Biol. 254: 392-403), chain shuffling (Marks et al. (1992) Biotechnology (NY) 10: 779-783), use of mutator strains of E. coli (Low et al. (1996) J. Mol. Biol. 260: 359-368), DNA shuffling (Patten et al. (1997) Curr. Opin. Biotechnol. 8: 724-733), phage display (Thompson et al. (1996) J. Mol. Biol. 256: 77-88), and sexual PCR (Crameri et al. (1998) Nature 391: 288-291). For immunotherapy applications, relevant functional assays include specific binding to human RAGE antigen, antibody internalization, and targeting to a tumor site(s) when administered to a tumor-bearing animal, as described herein below.

The present invention further provides cells and cell lines expressing anti-RAGE antibodies of the invention. Representative host cells include mammalian and human cells, such as CHO cells, HEK-293 cells, HeLa cells, CV-1 cells, and COS cells. Methods for generating a stable cell line following transformation of a heterologous construct into a host cell are known in the art. Representative non-mammalian host cells include insect cells (Potter et al. (1993) Int. Rev. Immunol. 10(2-3):103-112). Antibodies may also be produced in transgenic animals (Houdebine (2002) Curr. Opin. Biotechnol. 13(6):625-629) and transgenic plants (Schillberg et al. (2003) Cell Mol. Life Sci. 60(3):433-45).

As discussed above, monoclonal, chimeric and humanized antibodies, which have been modified by, e.g., deleting, adding, or substituting other portions of the antibody, e.g., the constant region, are also within the scope of the invention. For example, an antibody can be modified as follows: (i) by deleting the constant region; (ii) by replacing the constant region with another constant region, e.g., a constant region meant to increase half-life, stability or affinity of the antibody, or a constant region from another species or antibody class; or (iii) by modifying one or more amino acids in the constant region to alter, for example, the number of glycosylation sites, effector cell function, Fc receptor (FcR) binding, complement fixation, among others.

Methods for altering an antibody constant region are known in the art. Antibodies with altered function, e.g. altered affinity for an effector ligand, such as FcR on a cell, or the C1 component of complement can be produced by replacing at least one amino acid residue in the constant portion of the antibody with a different residue (see e.g., EP 388,151 A1, U.S. Pat. No. 5,624,821 and U.S. Pat. No. 5,648,260, the contents of all of which are hereby incorporated by reference). Similar type of alterations could be described which if applied to the murine, or other species immunoglobulin would reduce or eliminate these functions.

For example, it is possible to alter the affinity of an Fc region of an antibody (e.g., an IgG, such as a human IgG) for an FcR (e.g., FcγR1), or for C1q binding by replacing the specified residue(s) with a residue(s) having an appropriate functionality on its side chain, or by introducing a charged functional group, such as glutamate or aspartate, or perhaps an aromatic non-polar residue such as phenylalanine, tyrosine, tryptophan or alanine (see e.g., U.S. Pat. No. 5,624,821).

The antibody or binding fragment thereof may be conjugated with a cytotoxin, a therapeutic agent, or a radioactive metal ion. In one embodiment, the protein that is conjugated is an antibody or fragment thereof. A cytotoxin or cytotoxic agent includes any agent that is detrimental to cells. Non-limiting examples include, calicheamicin, taxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, puromycin, and analogs, or homologs thereof. Therapeutic agents include, but are not limited to, antimetabolites (e.g., methotrexate, 6-mercaptopurine, 6-thioguanine, cytarabine, and 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cis-dichlorodiamine platinum (II) (DDP), cisplatin), anthracyclines (e.g., daunorubicin and doxorubicin), antibiotics (e.g., dactinomycin, bleomycin, mithramycin, and anthramycin), and anti-mitotic agents (e.g., vincristine and vinblastine). Techniques for conjugating such moieties to proteins are well known in the art.

Alternatively, it is now possible to produce transgenic animals (e.g., mice) that are capable, upon immunization, of producing a full repertoire of human antibodies in the absence of endogenous immunoglobulin production. For example, it has been described that the homogeneous deletion of the antibody heavy-chain joining region (JM) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jackobovits et al., Proc. Natl. Acad. Sci. USA, 90:2551 (1993); Jakobovits et al., Nature, 362:255-258 (1993); Bruggermann et al., Year in Immune, 7:33 (1983); and Duchosal et al. Nature 355:258 (1992). Human antibodies can also be derived from phage-display libraries (Hoogenboom et al., J. Mol. Biol. 227:381 (1991); Marks et al., J. Mol. Biol., 222:581-597 (1991); Vaughan et al. Nature Biotech 14:309 (1996)).

In certain embodiments, antibodies of the present invention can be administered in combination with other agents as part of a combinatorial therapy. For example, in the case of inflammatory conditions, the subject antibodies can be administered in combination with one or more other agents useful in the treatment of inflammatory diseases or conditions. In the case of cardiovascular disease conditions, and particularly those arising from atherosclerotic plaques, which are thought to have a substantial inflammatory component, the subject antibodies can be administered in combination with one or more other agents useful in the treatment of cardiovascular diseases. In the case of cancer, the subject antibodies can be administered in combination with one or more anti-angiogenic factors, chemotherapeutics, or as an adjuvant to radiotherapy. It is further envisioned that the administration of the subject antibodies will serve as part of a cancer treatment regimen that may combine many different cancer therapeutic agents. In the case of IBD, the subject antibodies can be administered with one or more anti-inflammatory agents, and may additionally be combined with a modified dietary regimen.

Methods for Inhibiting an Interaction Between a RAGE-LBE and a RAGE-BP

The invention includes methods for inhibiting the interaction between RAGE and a RAGE-BP, or modulating RAGE activity. Preferably, such methods are used for treating RAGE-associated disorders.

Such methods may comprise administering an antibody raised to RAGE as disclosed herein. Such methods comprise administering an antibody that binds specifically to one or more epitopes of a RAGE protein having an amino acid sequence as set forth in SEQ ID NO:1, SEQ ID NO:3, SEQ ID NO:7, SEQ ID NO:9, SEQ ID NO:11, or SEQ ID NO:13. In yet another embodiment, such methods comprise administering a compound that inhibits the binding of RAGE to one or more RAGE-BPs. Exemplary methods of identifying such compounds are discussed below.

In certain embodiments, the interaction is inhibited in vitro, such as in a reaction mixture comprising purified proteins, cells, biological samples, tissues, artificial tissues, etc. In certain embodiments, the interaction is inhibited in vivo, for example, by administering an antibody that binds to RAGE or a RAGE-binding fragment thereof. The antibody or fragment thereof bind to RAGE and inhibit binding of a RAGE-BP.

The invention includes methods for preventing or treating a RAGE related disorder by inhibiting the interaction between RAGE and a RAGE-BP, or modulating RAGE activity. Such methods include administering an antibody to RAGE in an amount effective to inhibit the interaction and for a time sufficient to prevent or treat said disorder.

Nucleic Acids

Nucleic acids are deoxyribonucleotides or ribonucleotides and polymers thereof in single-stranded, double-stranded, or triplexed form. Unless specifically limited, nucleic acids may contain known analogues of natural nucleotides that have similar properties as the reference natural nucleic acid. Nucleic acids include genes, cDNAs, mRNAs, and cRNAs. Nucleic acids may be synthesized, or may be derived from any biological source, including any organism.

Representative nucleic acids of the invention comprise a nucleotide sequence encoding RAGE shown in any one of SEQ ID NOs: 6, 8, 10, 12, corresponding to disclosed cDNAs encoding RAGE of baboon, cynomologus monkey, and rabbit, or shown in SEQ ID NO: 15, corresponding to a genomic DNA sequence encoding baboon RAGE. Nucleic acids of the invention also comprise a nucleotide sequence encoding any of the antibody variable region amino acid sequences shown in SEQ ID NOs: 16-49.

Nucleic acids of the invention may also comprise a nucleotide sequence that is substantially identical to any one of SEQ ID NOs: 6, 8, 10, 12, and 15, including nucleotide sequences that are at least 90%, 91%, 92%, 93%, 94%, 95%, 96%, 97%, 98%, 99%, 99.5%, or 99.9% identical to any one of SEQ ID NOs: 6, 8, 10, 12, and 15.

Nucleic acids of the invention may also comprise a nucleotide sequence encoding a RAGE protein having an amino acid sequence that is substantially identical to any of the amino acid sequences shown in SEQ ID NOs: 7, 9, 11, and 13, including nucleotide sequences that are at least 90%, 91%, 92%, 93%, 94%, 95%, 96%, 97%, 98%, 99%, 99.5%, or 99.9% identical to any one of in SEQ ID NOs: 7, 9, 11, and 13.

Nucleic acids of the invention may also comprise a nucleotide sequence encoding an anti-RAGE antibody variable region having an amino acid sequence that is substantially identical to any of the amino acid sequences shown in SEQ ID NOs: 16-49, including a nucleotide sequence encoding an amino acid sequence that is at least 85%, 86%, 87%, 88%, 89%, 90%, 91%, 92%, 93%, 94%, 95%, 96%, 97%, 98%, 99%, 99.5%, or 99.9% identical to any of SEQ ID NOs: 16-49.

Sequences are compared for maximum correspondence using a sequence comparison algorithm using the full-length variable region encoding sequence of any one of SEQ ID NOs: 16-49, a nucleotide sequence encoding a full length variable region having any one of the sequences shown in SEQ ID NO: 16-49 as the query sequence, as described herein below, or by visual inspection.

Substantially identical sequences may be polymorphic sequences, i.e., alternative sequences or alleles in a population. An allelic difference may be as small as one base pair. Substantially identical sequences may also comprise mutagenized sequences, including sequences comprising silent mutations. A mutation may comprise one or more residue changes, a deletion of one or more residues, or an insertion of one or more additional residues.

Substantially identical nucleic acids are also identified as nucleic acids that hybridize specifically to or hybridize substantially to the full length of any one of SEQ ID NOs: 6, 8, 10, 12, or 15, or to the full length of any nucleotide sequence encoding a RAGE amino acid sequence shown in SEQ ID NOs: 7, 9, 11, and 13, or encoding an antibody variable region amino acid sequence shown in SEQ ID NOs: 16-49, under stringent conditions. In the context of nucleic acid hybridization, two nucleic acid sequences being compared may be designated a probe and a target. A probe is a reference nucleic acid molecule, and a target is a test nucleic acid molecule, often found within a heterogeneous population of nucleic acid molecules. A target sequence is synonymous with a test sequence.

For hybridization studies, useful probes are complementary to or mimic at least about 14 to 40 nucleotide sequence of a nucleic acid molecule of the present invention. Preferably, probes comprise 14 to 20 nucleotides, or even longer where desired, such as 30, 40, 50, 60, 100, 200, 300, or 500 nucleotides or up to the full length of any one of SEQ ID NOs: 6, 8, 10, 12, or 15, or to the full length of any nucleotide sequence encoding a RAGE amino acid sequence shown in SEQ ID NOs: 7, 9, 11, and 13, or encoding an antibody variable region amino acid sequence shown in SEQ ID NOs: 16-49. Such fragments may be readily prepared, for example, by chemical synthesis of the fragment, by application of nucleic acid amplification technology, or by introducing selected sequences into recombinant vectors for recombinant production.

Specific hybridization refers to the binding, duplexing, or hybridizing of a molecule only to a particular nucleotide sequence under stringent conditions when that sequence is present in a complex nucleic acid mixture (e.g., total cellular DNA or RNA). Specific hybridization may accommodate mismatches between the probe and the target sequence depending on the stringency of the hybridization conditions.

Stringent hybridization conditions and stringent hybridization wash conditions in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen (1993) Laboratory Techniques in Biochemistry and Molecular Biology-Hybridization with Nucleic Acid Probes, part I chapter 2, Elsevier, New York, N.Y. Generally, highly stringent hybridization and wash conditions are selected to be about 5° C. lower than the thermal melting point (Tₘ) for the specific sequence at a defined ionic strength and pH. Typically, under stringent conditions a probe will hybridize specifically to its target subsequence, but to no other sequences.

The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1×SSC at 65° C. An example of stringent wash conditions is 15 minutes in 0.2×SSC buffer at 65° C. See Sambrook et al., eds (1989) Molecular Cloning: A Laboratory Manual, Cold Spring Harbor Laboratory Press, Cold Spring Harbor, N.Y., for a description of SSC buffer. Often, a high stringency wash is preceded by a low stringency wash to remove background probe signal. An example of medium stringency wash conditions for a duplex of more than about 100 nucleotides, is 15 minutes in 1×SSC at 45° C. An example of low stringency wash for a duplex of more than about 100 nucleotides, is 15 minutes in 4× to 6×SSC at 40° C. For short probes (e.g., about 10 to 50 nucleotides), stringent conditions typically involve salt concentrations of less than about 1M Na⁺ ion, typically about 0.01 to 1M Na⁺ ion concentration (or other salts) at pH 7.0-8.3, and the temperature is typically at least about 30° C. Stringent conditions may also be achieved with the addition of destabilizing agents such as formamide. In general, a signal to noise ratio of 2-fold (or higher) than that observed for an unrelated probe in the particular hybridization assay indicates detection of a specific hybridization.

The following are examples of hybridization and wash conditions that may be used to identify nucleotide sequences that are substantially identical to reference nucleotide sequences of the present invention: a probe nucleotide sequence preferably hybridizes to a target nucleotide sequence in 7% sodium dodecyl sulphate (SDS), 0.5M NaPO₄, 1 mM EDTA at 50° C. followed by washing in 2×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.5×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 50° C.; more preferably, a probe and target sequence hybridize in 7% sodium dodecyl sulphate (SDS), 0.5M NaPO₄, 1 mM EDTA at 50° C. followed by washing in 0.1×SSC, 0.1% SDS at 65° C.

A further indication that two nucleic acid sequences are substantially identical is that proteins encoded by the nucleic acids are substantially identical, share an overall three-dimensional structure, or are biologically functional equivalents. These terms are defined further herein below. Nucleic acid molecules that do not hybridize to each other under stringent conditions are still substantially identical if the corresponding proteins are substantially identical. This may occur, for example, when two nucleotide sequences comprise conservatively substituted variants as permitted by the genetic code.

Conservatively substituted variants are nucleic acid sequences having degenerate codon substitutions wherein the third position of one or more selected (or all) codons is substituted with mixed-base and/or deoxyinosine residues. See Batzer et al. (1991) Nucleic Acids Res. 19:5081; Ohtsuka et al. (1985) J. Biol. Chem. 260:2605-2608; and Rossolini et al. (1994) Mol. Cell Probes 8:91-98.

Nucleic acids of the invention also comprise nucleic acids complementary to any one of SEQ ID NOs: 6, 8, 10, 12, or 15, or nucleotide sequences encoding a RAGE amino acid sequence shown in SEQ ID NOs: 7, 9, 11, and 13, or encoding an antibody variable region amino acid sequence shown in SEQ ID NOs: 16-49, and complementary sequences thereof. Complementary sequences are two nucleotide sequences that comprise antiparallel nucleotide sequences capable of pairing with one another upon formation of hydrogen bonds between base pairs. As used herein, the term complementary sequences means nucleotide sequences which are substantially complementary, as may be assessed by the same nucleotide comparison methods set forth below, or is defined as being capable of hybridizing to the nucleic acid segment in question under relatively stringent conditions such as those described herein. A particular example of a complementary nucleic acid segment is an antisense oligonucleotide.

A subsequence is a sequence of nucleic acids that comprises a part of a longer nucleic acid sequence. An exemplary subsequence is a probe, described herein above, or a primer. The term primer as used herein refers to a contiguous sequence comprising about 8 or more deoxyribonucleotides or ribonucleotides, preferably 10-20 nucleotides, and more preferably 20-30 nucleotides of a selected nucleic acid molecule. The primers of the invention encompass oligonucleotides of sufficient length and appropriate sequence so as to provide initiation of polymerization on a nucleic acid molecule of the present invention.

An elongated sequence comprises additional nucleotides (or other analogous molecules) incorporated into the nucleic acid. For example, a polymerase (e.g., a DNA polymerase) may add sequences at the 3′ terminus of the nucleic acid molecule. In addition, the nucleotide sequence may be combined with other DNA sequences, such as promoters, promoter regions, enhancers, polyadenylation signals, intronic sequences, additional restriction enzyme sites, multiple cloning sites, and other coding segments. Thus, the invention also provides vectors comprising the disclosed nucleic acids, including vectors for recombinant expression, wherein a nucleic acid of the invention is operatively linked to a functional promoter. When operatively linked to a nucleic acid, a promoter is in functional combination with the nucleic acid such that the transcription of the nucleic acid is controlled and regulated by the promoter region. Vectors refer to nucleic acids capable of replication in a host cell, such as plasmids, cosmids, and viral vectors.

Nucleic acids of the present invention may be cloned, synthesized, altered, mutagenized, or combinations thereof. Standard recombinant DNA and molecular cloning techniques used to isolate nucleic acids are known in the art. Site-specific mutagenesis to create base pair changes, deletions, or small insertions is also known in the art. See e.g., Sambrook et al. (eds.) (1989) Molecular Cloning: A Laboratory Manual. Cold Spring Harbor Laboratory Press, Cold Spring Harbor, N.Y.; Silhavy et al. (1984) Experiments with Gene Fusions. Cold Spring Harbor Laboratory Press, Cold Spring Harbor, N.Y.; Glover & Hames (1995) DNA Cloning: A Practical Approach, 2nd ed. IRL Press at Oxford University Press, Oxford/N.Y.; Ausubel (ed.) (1995) Short Protocols in Molecular Biology, 3rd ed. Wiley, N.Y.

Prophylactic and Therapeutic Methods

The present invention provides a method for treating a subject having a disease or disorder characterized by amyloid deposit of Aβ, such as Alzheimer's disease, which comprises administering a therapeutically effective amount of an antibody that binds specifically to RAGE and inhibits the binding of a RAGE binding partner to a subject.

The invention also provides a method of inhibiting or reducing accumulation of amyloid deposit of Aβ in a subject, comprising administering to the subject an effective amount of an antibody that binds specifically to RAGE and inhibits the binding of a RAGE binding partner. Also included within the invention is a method of inhibiting or reducing neurodegeneration in a subject, comprising administering to the subject an effective amount of an antibody that binds specifically to RAGE and inhibits the binding of a RAGE binding partner. The invention further includes a method of inhibiting or reducing cognitive decline, or improving cognition, in a subject, comprising administering to the subject an effective amount of an antibody that binds specifically to RAGE and inhibits the binding of a RAGE binding partner. The invention also provides a method for treating a subject having an amyloidogenic disease or disorder characterized by amyloid deposit which comprises administering a therapeutically effective amount of an antibody that binds specifically to RAGE and inhibits the binding of a RAGE binding partner.

The present invention provides a method for treating a subject having a disease or disorder characterized by amyloid deposit of Aβ, such as Alzheimer's disease, which comprises administering a therapeutically effective amount of an antibody that binds specifically to RAGE and inhibits the binding of a RAGE binding partner to a subject under conditions that generate a beneficial therapeutic response in the subject (e.g., reduction of plaque burden, inhibition of plaque formation, reduction of neuritic dystrophy, and improvement of cognitive function, e.g., rapidly improving cognition, and/or reversing, treating or preventing cognitive decline) in the patient.

Diseases associated with amyloid deposits of Aβ in the brain include Alzheimer's disease, Down's syndrome and cognitive impairment. The latter can occur with or without other characteristics of an amyloidogenic disease.

In addition to advanced glycation end products (AGE's), which form in prolonged hyperglycemic states, the ligands of RAGE include proteins having β-sheet fibrillar structure that are characteristic of amyloid deposits and pro-inflammatory mediators, including beta-Amyloid protein (Aβ), serum amyloid (SAA) (fibrillar form), S100/calgranulins (e.g., S100A12, S100B, S100A8-A9), and high mobility group box-1 chromosomal protein 1 (HMGB1, also known as amphoterin). There is growing awareness of the role of RAGE in the pathological progression of amyloidogenic diseases. In addition to its contributing to the pathogenesis of Alzheimer's disease, RAGE has been shown to be closely linked to cell stress and deposition of serum amyloid A (SAA) in spleen (Yan et al., 2000, Nature Med., 6:643-51). RAGE is associated with the accumulation of amyloid in kidneys and the tissue destruction leading to kidney failure of individuals with familial amyloidotic polyneuropathy (FAP) (Matsunaga et al., 2005, Scand. J. Clin. Lab. Invest.). The RAGE ligand amphoterin (HMGB1) also contains an amyloidogenic peptide—one that is highly homologous to the Alzheimer's Aβ peptide and forms amyloid-like peptides when released from the native protein (Kallijarvi et al, 2001, Biochem., 40:10032-7).

The interaction of Aβ with RAGE-bearing cells in the walls of blood vessels results in transport of Aβ across the blood-brain barrier (BBB) and expression of pro-inflammatory cytokines and endothelin-1 (ET-1), the latter mediating Aβ-induced vasoconstriction. Thus, the present invention also provides methods for reducing Aβ-induced vasoconstriction.

The inhibition of RAGE-ligand interaction has been shown to suppress the accumulation of Aβ in brain parenchyma in a transgenic mouse model for Alzheimer's-like disease (Deane et al., 2003, Nature Medicine 9:907-913). The active, pathogenic role of RAGE in a wide range of amyloidogenic diseases and disorders makes it possible to provide therapeutic, beneficial treatment to patients with these amyloidogenic disorders by the method of the present invention, which provides antibodies that bind specifically to RAGe and inhibit the binding of a RAGE binding partner.

The methods of the invention can be used on both asymptomatic patients and those currently showing symptoms of disease. The antibodies used in such methods can be human, humanized, chimeric or nonhuman antibodies, or fragments thereof (e.g., RAGE binding fragments), as described herein. In yet another aspect, the invention features administering antibodies prepared from a human immunized with [I assume this should be RAGE, although perhaps it should simply be deleted] Aβ peptide, which human can be the patient to be treated with antibody. The therapeutic methods of the invention can be performed using an antibody that

(a) competes for binding to RAGE with an antibody selected from the group consisting of XT-H1, XT-H2, XT-H3, XT-H5, XT-H7, and XT-M4;

(b) binds to an epitope of RAGE that is bound by an antibody selected from the group consisting of XT-H1, XT-H2, XT-H3, XT-H5, XT-H7, and XT-M4;

(c) comprises one or more complementarity determining regions (CDRs) of a light chain or heavy chain of an antibody selected from the group consisting of XT-H1, XT-H2, XT-H3, XT-H5, XT-H7, and XT-M4; or

(d) is a RAGE-binding fragment of an antibody according to (a), (b) or (c).

For example, the methods of the invention can be performed by administering to the subject an antibody or RAGE-binding antibody fragment that comprises a light chain variable region comprising CDRs of a XT-M4 light chain variable region (SEQ ID NO: 17), a heavy chain variable region comprising CDRs of a XT-M4 heavy chain variable region sequence (SEQ ID NO: 16), a human kappa light chain constant region; and a human IgG1 heavy chain constant region. The methods of the invention can also be performed using an antibody or RAGE-binding fragment thereof comprises a light chain variable region having the amino acid sequence of a XT-M4 light chain variable region (SEQ ID NO: 17), a heavy chain variable region having the amino acid sequence of a XT-M4 heavy chain variable region sequence (SEQ ID NO: 16), a human kappa light chain constant region; and a human IgG1 heavy chain constant region. Descriptions of these and many other antibodies that can be used successfully for the method of the invention are described herein.

Therapeutic agents of the invention are typically substantially pure from undesired contaminant. This means that an agent is typically at least about 50% w/w (weight/weight) pure, as well as being substantially free from interfering proteins and contaminants. Sometimes the agents are at least about 80% w/w and, more preferably at least 90 or about 95% w/w pure. However, using conventional protein purification techniques, homogeneous peptides of at least 99% w/w pure can be obtained.

The invention includes administering an antibody with a pharmaceutical carrier as a pharmaceutical composition. Alternatively, the antibody can be administered to a patient by administering a polynucleotide encoding at least one antibody chain. The polynucleotide is expressed to produce the antibody chain in the patient. The polynucleotide can encode heavy and light chains of the antibody. The polynucleotide is expressed to produce the heavy and light chains in the patient. In exemplary embodiments, the patient is monitored for level of administered antibody in the blood of the patient.

The invention thus fulfills a longstanding need for therapeutic regimes for preventing or ameliorating the neuropathology and, in some patients, the cognitive impairment associated with Alzheimer's disease.

Reducing Cognitive Decline and/or Improving Cognition

The present invention provides a method for inhibiting or reducing cognitive decline, and/or improving cognition, in a patient having or at risk for an suffering from an Aβ-related disease or disorder or amyloidogenic disease or disorder (e.g., AD), comprising administering to the subject an effective amount of an antibody that binds specifically to RAGE and inhibits the binding of a RAGE binding partner.

The methods feature administering an effective dose of an antibody of the invention such that cognitive decline is reduced, and/or improvement in cognition is achieved. For example, improvement in one or more cognitive deficits in the patient (e.g., procedural learning and/or memory, deficits) is achieved. The cognitive deficit can be an impairment in explicit memory (also known as “declarative” or “working” memory), which is defined as the ability to store and retrieve specific information that is available to consciousness and which can therefore be expressed by language (e.g. the ability to remember a specific fact or event). Alternatively, the cognitive deficit can be an impairment in procedural memory (also known as “implicit” or “contextual” memory), which is defined as the ability to acquire, retain, and retrieve general information or knowledge that is not available to consciousness and which requires the learning of skills, associations, habits, or complex reflexes to be expressed, e.g. the ability to remember how to execute a specific task. Individuals suffering from procedural memory deficits are much more impaired in their ability to function normally. As such, treatments which are effective in improving deficits in procedural memory are highly desirable and advantageous.

Patients Amenable to Treatment

Patients amenable to treatment by the invention include individuals at risk of an Aβ-related disease or disorder or amyloidogenic disease or disorder but not showing symptoms, as well as patients presently showing symptoms. In the case of Alzheimer's disease, virtually anyone is at risk of suffering from Alzheimer's disease if he or she lives long enough. Therefore, the present methods can be administered prophylactically to the general population without the need for any assessment of the risk of the subject patient.

The present methods are especially useful for individuals who are at risk for AD, e.g., those who exhibit risk factors of AD. The main risk factor for AD is increased age. As the population ages, the frequency of AD continues to increase. Current estimates indicate that up to 10% of the population over the age of 65 and up to 50% of the population over the age of 85 have AD.

Although rare, certain individuals can be identified at an early age as being genetically predisposed to developing AD. Individuals carrying the heritable form of AD, known as “familial AD” or “early-onset AD”, can be identified from a well documented family history of AD, of the analysis of a gene that is known to confer AD when mutated, for example the APP or presenilin gene. Well characterized APP mutations include the “Hardy” mutations at codons 716 and 717 of APP770 (e.g., valine.sup.717 to isoleucine (Goate et al., (1991), Nature 349:704); valine.sup.717 to glycine (Chartier et al. (1991) Nature 353:844; Murrell et al. (1991), Science 254:97); valine.sup.717 to phenylalanine (Mullan et al. (1992), Nature Genet. 1:345-7)), the “Swedish” mutations at codon 670 and 671 of APP770, and the “Flemish” mutation at codon 692 of APP770. Such mutations are thought to cause Alzheimer's disease by increased or altered processing of APP to Aβ, particularly processing of APP to increased amounts of the long form of Aβ (i.e., Aβ1-42 and Aβ 1-43). Mutations in other genes, such as the presenilin genes, PS1 and PS2, are thought indirectly to affect processing of APP to generate increased amounts of long form Aβ (see Hardy, TINS 20: 154 (1997); Kowalska et al., (2004), Polish J. Pharmacol., 56: 171-8). In addition to AD, mutations at amino acid 692 or 693 of the 770-amino acid isoform of APP have been implicated in cerebral amyloidogenic disorder called Hereditary Cerebral Hemorrhage with Amyloidosis of the Dutch-type (HCHWA-D).

More commonly, AD is not inherited by a patient but develops due to the complex interplay of a variety of genetic factors. These individuals are said to have “sporadic AD” (also known as “late-onset AD”), a form which is much more difficult to diagnose. Nonetheless, the patient population can be screened for the presence of susceptibility alleles or traits that do not cause AD but are known to segregate with AD at a higher frequency than in the general population, e.g., the ε2, ε 3, and ε 4 alleles of apolipoprotein E (Corder et. al. (1993), Science, 261: 921-923). In particular, patients lacking the ε 4 allele, preferably in addition to some other marker for AD, may be identified as “at risk” for AD. For example, patients lacking the ε 4 allele who have relatives who have AD or who suffer from hypercholesterolemia or atherosclerosis may be identified as “at risk” for AD. Another potential biomarker is the combined assessment of cerebral spinal fluid (CSF) Aβ42 and tau levels. Low Aβ42 and high tau levels have a predictive value in identifying patients at risk for AD.

Other indicators of patients at risk for AD include in vivo dynamic neuropathological data, for example, in vivo detection of brain beta amyloid, patterns of brain activation, etc. Such data can be obtained using, for example, three-dimensional magnetic resonance imaging (MRI), positron emission tomography (PET) scan and single-photon emission computed tomography (SPECT). Indicators of patients having probable AD include, but are not limited to, patients (1) having dementia, (2) of an age of 40-90 years old, (3) cognitive deficits, e.g., in two or more cognitive domains, (4) progression of deficits for more than six months, (5) consciousness undisturbed, and/or (6) absence of other reasonable diagnoses.

Individuals suffering either sporadic or familial forms of AD are usually, however, diagnosed following presentation of one or more characteristic symptoms of AD. Common symptoms of AD include cognitive deficits that affect the performance of routine skills or tasks, problems with language, disorientation to time or place, poor or decreased judgment, impairments in abstract thought, loss of motor control, mood or behavior alteration, personality change, or loss of initiative. The number deficits or the degree of the cognitive deficit displayed by the patient usually reflects the extent to which the disease has progressed. For example, the patient may exhibit only a mild cognitive impairment, such that the patient exhibits problems with memory (e.g. contextual memory) but is otherwise able to function well.

The present methods are also useful for individuals who have an Aβ-related cognitive deficit, e.g. Aβ-related dementia. In particular, the present methods are especially useful for individuals who have a cognitive deficit or aberrancy caused by or attributed to the presence of soluble oligomeric Aβ in the central nervous system (CNS), for example, in the brain or CSF. Cognitive deficits caused by or associated with Aβ also include those caused by or associated with: (1) the development of β-amyloid plaques in the brain; (2) abnormal rates of Aβ synthesis, processing, degradation or clearance; (3) the formation or activity of soluble oligomeric Aβ species (e.g., in the brain); and/or (4) the formation of abnormal forms of Aβ. It is not necessary that an actual causative link be established between an Aβ abnormality and cognitive deficit in a particular patient, however, some the link should be indicated, for example, by one of the above-described markers of AD to distinguish patients suffering from non-Aβ related cognitive deficits who would not be expected to benefit from treatment with an Aβ immunotherapeutic agent.

Several tests have been developed to assess cognitive skills or performance in human subjects, for example, subjects at risk for or having symptoms or pathology of dementia disorders (e.g., AD). Cognitive deficits can be identified by impaired performance of these tests, and many treatments have been proposed based on their ability to improve performance in these tests. Although some tasks have evaluated behaviors or motor function of subjects, most tasks have been designed to test learning or memory.

Cognition in humans may be assessed using a wide variety of tests including, but not limited to, the following tests. The ADAS-Cog (Alzheimer Disease Assessment Scale-Cognitive) is 11-part test that takes 30 minutes to complete. The ADAS-Cog is a preferred brief exam for the study of language and memory skills. See Rosen et al. (1984) Am J Psychiatry. 141(11):1356-64; Ihl et al. (2000) Neuropsychobiol. 41(2):102-7; and Weyer et al. (1997) Int Psychogeriatr. 9(2):123-38.

The Blessed Test is another quick (˜10 minute) test of cognition which assesses activities of daily living and memory, concentration and orientation. See Blessed et al. (1968) Br J Psychiatry 114(512):797-811.

The Cambridge Neuropsychological Test Automated Battery (CANTAB) is used for the assessment of cognitive deficits in humans with neurodegenerative diseases or brain damage. It consists of thirteen interrelated computerized tests of memory, attention, and executive function, and is administered via a touch sensitive screen from a personal computer. The tests are language and largely culture free, and have shown to be highly sensitive in the early detection and routine screening of Alzheimer's disease. See Swainson et al. (2001) Dement Geriatr Cogn Disord.; 12:265-280; and Fray and Robbins (1996) Neurotoxicol Teratol. 18(4):499-504. Robbins et al. (1994) Dementia 5(5):266-81.

The Consortium to Establish a Registry for Alzheimer's Disease (CERAD) Clinical and Neuropsychological Tests include a verbal fluency test, Boston Naming Test, Mini Mental State Exam (MMSE), ten-item word recall, constructional praxis, and delayed recall of praxis items. The test typically takes 20-30 minutes and is convenient and effective at assessing and tracking cognitive decline. See Morris et al. (1988) Psychopharmacol Bull. 24(4):641-52; Morris et al. (1989) Neurology 39(9):1159-65; and Welsh et al. (1991) Arch Neurol. 48(3):278-81.

The Mini Mental State Exam (MMSE) developed in 1975 by Folestein et al, is a brief test of mental status and cognition function. It does not measure other mental phenomena and is therefore not a substitute for a full mental status examination. It is useful in screening for dementia and its scoring system is helpful in following progress over time. The Mini-Mental State Examination MMSE is widely used, with norms adjusted for age and education. It can be used to screen for cognitive impairment, to estimate the severity of cognitive impairment at a given point in time, to follow the course of cognitive changes in an individual over time, and to document an individual's response to treatment. Cognitive assessment of subjects may require formal neuropsychologic testing, with follow-up testing separated by nine months or more (in humans). See Folstein et al. (1975) J Psychiatr Res. 12:196-198; Cockrell and Folstein (1988) Psychopharm Bull. 24(4):689-692; and Crum et al. (1993) J. Am. Med. Association 18:2386-2391.

The Seven-Minute Screen is a screening tool to help identify patients who should be evaluated for Alzheimer's disease. The screening tool is highly sensitive to the early signs of AD, using a series of questions to assess different types of intellectual functionality. The test consists of 4 sets of questions that focus on orientation, memory, visuospatial skills and expressive language. It can distinguish between cognitive changes due to the normal aging process and cognitive deficits due to dementia. See Solomon and Pendlebury (1998) Fam Med. 30(4):265-71, Solomon et al. (1998) Arch Neurol. 55(3):349-55.

Individuals presently suffering from Alzheimer's disease can be recognized from characteristic dementia, as well as the presence of risk factors described above. In addition, a number of diagnostic tests are available for identifying individuals who have AD. These include measurement of CSF tau and Aβ42 levels. Elevated tau and decreased Aβ42 levels signify the presence of AD. Individuals suffering from Alzheimer's disease can also be diagnosed by ADRDA criteria.

Combination Therapy

The anti-RAGE antibodies of the present invention may be used in combination with one or more additional agents, which may be administered to a subject concurrently or sequentially in either order. The disclosed combination therapies may elicit a synergistic therapeutic effect, i.e, an effect greater than the effect of either agent alone. Measurable therapeutic effects are described herein above. For example, a synergistic therapeutic effect may be an effect of at least about two-fold greater than the therapeutic effect elicited by a single agent, or at least about at least about five-fold greater, or at least about ten-fold greater, or at least about twenty-fold greater, or at least about fifty-fold greater, or at least about one hundred-fold greater

For example, the invention includes administering a therapeutically effective amount of an antibody that binds specifically to RAGE and inhibits the binding of a RAGE binding partner in combination with another antibody that binds specifically to Aβ. The antibody that binds to Aβ can be an antibody that specifically binds to Aβ peptide without binding to full-length amyloid precursor protein (APP). Alternatively, the antibody of the invention may be administered in combination with antibodies that bind to and/or capture soluble Aβ, or that bind to an amyloid deposit in the patient and induce a clearing response against the amyloid deposit. Such a clearing response can be effected by Fc receptor mediated phagocytosis. Such a clearing response can be engineered into an antibody, for example, by including an Fc receptor-binding domain (e.g., an IgG2a constant region). The antibody of the invention can also be administered to a patient who has received or is receiving an Aβ vaccine. In the case of Alzheimer's and Down's syndrome, in which amyloid deposits occur in the brain, antibodies of the invention can also be administered in conjunction with other agents that increase passage of the agents of the invention across the blood-brain barrier. Antibodies of the invention can also be administered in combination with other agents that enhance access of the therapeutic agent to a target cell or tissue, for example, liposomes and the like. Coadministering such agents can decrease the dosage of a therapeutic agent (e.g., therapeutic antibody or antibody chain) needed to achieve a desired effect.

Monitoring the Course of Treatment

The invention provides methods of monitoring treatment in a patient suffering from or susceptible to Alzheimer's, i.e., for monitoring a course of treatment being administered to a patient. The methods can be used to monitor both therapeutic treatment on symptomatic patients and prophylactic treatment on asymptomatic patients. In particular, the methods are useful for monitoring passive immunization (e.g., measuring level of administered antibody).

Some methods involve determining a baseline value, for example, of an antibody level or profile in a patient, before administering a dosage of agent, and comparing this with a value for the profile or level after treatment. A significant increase (i.e., greater than the typical margin of experimental error in repeat measurements of the same sample, expressed as one standard deviation from the mean of such measurements) in value of the level or profile signals a positive treatment outcome (i.e., that administration of the agent has achieved a desired response). If the value for immune response does not change significantly, or decreases, a negative treatment outcome is indicated.

In other methods, a control value (i.e., a mean and standard deviation) of level or profile is determined for a control population. Typically the individuals in the control population have not received prior treatment. Measured values of the level or profile in a patient after administering a therapeutic agent are then compared with the control value. A significant increase relative to the control value (e.g., greater than one standard deviation from the mean) signals a positive or sufficient treatment outcome. A lack of significant increase or a decrease signals a negative or insufficient treatment outcome. Administration of agent is generally continued while the level is increasing relative to the control value. As before, attainment of a plateau relative to control values is an indicator that the administration of treatment can be discontinued or reduced in dosage and/or frequency.

In other methods, a control value of the level or profile (e.g., a mean and standard deviation) is determined from a control population of individuals who have undergone treatment with a therapeutic agent and whose levels or profiles have plateaued in response to treatment. Measured values of levels or profiles in a patient are compared with the control value. If the measured level in a patient is not significantly different (e.g., more than one standard deviation) from the control value, treatment can be discontinued. If the level in a patient is significantly below the control value, continued administration of agent is warranted. If the level in the patient persists below the control value, then a change in treatment may be indicated.

In other methods, a patient who is not presently receiving treatment but has undergone a previous course of treatment is monitored for antibody levels or profiles to determine whether a resumption of treatment is required. The measured level or profile in the patient can be compared with a value previously achieved in the patient after a previous course of treatment. A significant decrease relative to the previous measurement (i.e., greater than a typical margin of error in repeat measurements of the same sample) is an indication that treatment can be resumed. Alternatively, the value measured in a patient can be compared with a control value (mean plus standard deviation) determined in a population of patients after undergoing a course of treatment. Alternatively, the measured value in a patient can be compared with a control value in populations of prophylactically treated patients who remain free of symptoms of disease, or populations of therapeutically treated patients who show amelioration of disease characteristics. In all of these cases, a significant decrease relative to the control level (i.e., more than a standard deviation) is an indicator that treatment should be resumed in a patient.

The tissue sample for analysis is typically blood, plasma, serum, mucous fluid or cerebrospinal fluid from the patient. The sample is analyzed, for example, for levels or profiles of antibodies to RAGE peptide, e.g., levels or profiles of humanized antibodies. ELISA methods of detecting antibodies specific to RAGE are described in the Examples. In some methods, the level or profile of an administered antibody is determined using a clearing assay, for example, in an in vitro phagocytosis assay, as described herein. In such methods, a tissue sample from a patient being tested is contacted with amyloid deposits (e.g., from a PDAPP mouse) and phagocytic cells bearing Fc receptors. Subsequent clearing of the amyloid deposit is then monitored. The existence and extent of clearing response provides an indication of the existence and level of antibodies effective to clear Aβ in the tissue sample of the patient under test.

The antibody profile following passive immunization typically shows an immediate peak in antibody concentration followed by an exponential decay. Without a further dosage, the decay approaches pretreatment levels within a period of days to months depending on the half-life of the antibody administered.

In some methods, a baseline measurement of antibody to RAGE in the patient is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dosage of antibody is administered. In some methods, peak or subsequent measured levels less background are compared with reference levels previously determined to constitute a beneficial prophylactic or therapeutic treatment regime in other patients. If the measured antibody level is significantly less than a reference level (e.g., less than the mean minus one standard deviation of the reference value in population of patients benefiting from treatment) administration of an additional dosage of antibody is indicated.

Measurable indices for monitoring the course of treatment and a patient's status include monitoring (reduction in) of levels of Aβ in the patient's brain, monitoring the amyloid sink, and monitoring amelioration of Aβ-induced deficits in neuronal function. Other measurable indices include monitoring the status or changes in vascular congophilic amyloid angiopathy (CAA) pathology in Alzheimer's disease, and monitoring the patient include changes in intracellular signaling and inflammation mediated by Aβ. The latter will provide information relating to regulation of RAGE by its ligands with multiple divergent signaling pathways, e.g., activation of transcriptional factor NF-kB, activates a RAGE promoter and neuroxic response is mediated by activation cell signaling (MAP Kinase cascade (MAPKs), ERK1/2, Akt, JNK, p38) and anti-RAGE MAbs block phosphorylation of JNK, p38, NFkB. Useful measures also include monitoring Aβ-induced signaling & synaptic plasticity potentiated by RAGE, and differential brain influx/efflux of Aβ across the blood/brain barrier, mediated by RAGE and LRP, may mediate differential brain influx/efflux of Aβ across blood brain barrier. The present invention thus provides methods for reducing intracellular signaling (e.g., reducing the MAPK cascade) and inflammation associated with Aβ.

Additional methods include monitoring, over the course of treatment, any art-recognized physiologic symptom (e.g., physical or mental symptom) routinely relied on by researchers or physicians to diagnose or monitor amyloidogenic diseases (e.g., Alzheimer's disease). For example, one can monitor cognitive impairment. The latter is a symptom of Alzheimer's disease and Down's syndrome but can also occur without other characteristics of either of these diseases. For example, cognitive impairment can be monitored by determining a patient's score on the Mini-Mental State Exam in accordance with convention throughout the course of treatment.

Pharmaceutical Preparations

The subject proteins or nucleic acids of the present invention are most preferably administered in the form of appropriate compositions. As appropriate compositions there may be cited all compositions usually employed for systemically or locally administering drugs. The pharmaceutically acceptable carrier should be substantially inert, so as not to act with the active component. Suitable inert carriers include water, alcohol, polyethylene glycol, mineral oil or petroleum gel, propylene glycol, phosphate buffer saline (PBS), baceriostatic water for injection (BWFI), sterile water for injection (SWFI), and the like. Said pharmaceutical preparations (including the subject antibodies or nucleic acids encoding the subject antibodies) may be formulated for administration in any convenient way for use in human or veterinary medicine.

Thus, another aspect of the present invention provides pharmaceutically acceptable compositions comprising an effective amount of an antibody, formulated together with one or more pharmaceutically acceptable carriers (additives) and/or diluents. As described in detail below, the pharmaceutical compositions of the present invention may be specially formulated for administration in solid or liquid form, including those adapted for the following: (1) oral administration, for example, drenches (aqueous or non-aqueous solutions or suspensions), tablets, boluses, powders, granules, pastes for application to the tongue; (2) parenteral administration, for example, by subcutaneous, intramuscular or intravenous injection as, for example, a sterile solution or suspension; (3) topical application, for example, as a cream, ointment or spray applied to the skin; or (4) intravaginally or intrarectally, for example, as a pessary, cream or foam. However, in certain embodiments the subject agents may be simply dissolved or suspended in sterile water. In certain embodiments, the pharmaceutical preparation is non-pyrogenic, i.e., does not elevate the body temperature of a patient. Parenteral administration, in particular subcutaneous and intravenous injection, is the preferred route of administration.

In certain embodiments, one or more agents may contain a basic functional group, such as amino or alkylamino, and are, therefore, capable of forming pharmaceutically acceptable salts with pharmaceutically acceptable acids. The term “pharmaceutically acceptable salts” in this respect, refers to the relatively non-toxic, inorganic and organic acid addition salts of compounds of the present invention. These salts can be prepared in situ during the final isolation and purification of the compounds of the invention, or by separately reacting a purified compound of the invention in its free base form with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, and laurylsulphonate salts and the like. (See, for example, Berge et al. (1977) “Pharmaceutical Salts,” J. Pharm. Sci. 66: 1-19).

The pharmaceutically acceptable salts of the agents include the conventional nontoxic salts or quaternary ammonium salts of the compounds, e.g., from non-toxic organic or inorganic acids. For example, such conventional nontoxic salts include those derived from inorganic acids such as hydrochloride, hydrobromic, sulfuric, sulfamic, phosphoric, nitric, and the like; and the salts prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

In other cases, the one or more agents may contain one or more acidic functional groups and, thus, are capable of forming pharmaceutically acceptable salts with pharmaceutically acceptable bases. These salts can likewise be prepared in situ during the final isolation and purification of the compounds, or by separately reacting the purified compound in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. (see, for example, Berge et al., supra)

Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: (1) water soluble antioxidants, such as ascorbic acid, cysteine hydrochloride, sodium bisulfate, sodium metabisulfite, sodium sulfite and the like, (2) oil-soluble antioxidants, such as ascorbyl palpitate, butylated hydroxyanisole (BHA), butylated hydroxytoluene (BHT), lecithin, propyl gallate, alpha-tocopherol, and the like, and (3) metal chelating agents, such as citric acid, ethylenediamine tetraacetic acid (EDTA), sorbitol, tartaric s acid, phosphoric acid, and the like.

Formulations of the present invention include those suitable for oral, nasal, topical (including buccal and sublingual), rectal, vaginal and/or parenteral administration. The formulations may conveniently be presented in unit dosage form and may be prepared by any methods well known in the art of pharmacy. The amount of active ingredient which can be combined with a carrier material to produce a single dosage form will vary depending upon the host being treated, the particular mode of administration, etc. The amount of active ingredient that can be combined with a carrier material to produce a single dosage form will generally be that amount of the compound that produces a therapeutic effect. Generally, out of one hundred percent, this amount will range frown about 1 percent to about ninety-nine percent of active ingredient, preferably from about 5 percent to about 70 percent, most preferably from about 10 percent to about 30 percent.

Methods of preparing these formulations or compositions include the step of bringing into association an agent with the carrier and, optionally, one or more accessory ingredients. In general, the formulations are prepared by uniformly and intimately bringing into association an agent of the present invention with liquid carriers, or timely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a compound of the present invention as an active ingredient. A compound of the present invention may also be administered as a bolus, electuary or paste.

In solid dosage forms of the invention for oral administration (capsules, tablets, pills, dragees, powders, granules and the like), the active ingredient is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions that can be dissolved in sterile water, or some other sterile injectable medium immediately before use. These compositions may also optionally contain opacifying agents and may be of a composition that they release the active ingredient(s) only, or preferentially, in a certain portion of the gastrointestinal tract, optionally, in a delayed manner. Examples of embedding compositions that can be used include polymeric substances and waxes. The active ingredient can also be in micro-encapsulated form, if appropriate, with one or more of the above-described excipients.

Liquid dosage forms for oral administration of the compounds of the invention include pharmaceutically acceptable emulsions, microemulsions, solutions, suspensions, syrups and elixirs. In addition to the active ingredient, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar and tragacanth, and mixtures thereof.

Formulations of the pharmaceutical compositions of the invention for rectal or vaginal administration may be presented as a suppository, which may be prepared by mixing one or more compounds of the invention with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the agents.

Formulations of the present invention which are suitable for vaginal administration also include pessaries, tampons, creams, gels, pastes, foams or spray formulations containing such carriers as are known in the art to be appropriate.

Dosage forms for the topical or transdermal administration of a compound of this invention include powders, sprays, ointments, pastes, creams, lotions, gels, solutions, patches and inhalants. The active compound may be mixed under sterile conditions with a pharmaceutically acceptable carrier, and with any preservatives, buffers, or propellants that may be required.

The ointments, pastes, creams and gels may contain, in addition to an active compound of this invention, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to a compound of this invention, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the agents in the proper medium. Absorption enhancers can also be used to increase the flux of the agents across the slain. The rate of such flux can be controlled by either providing a rate controlling membrane or dispersing the compound in a polymer matrix or gel.

Ophthalmic formulations, eye ointments, powders, solutions and the like, are also contemplated as being within the scope of this invention.

Pharmaceutical compositions of this invention suitable for parenteral administration comprise one or more compounds of the invention in combination with one or more pharmaceutically acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents that delay absorption such as aluminum monostearate and gelatin.

In some cases, in order to prolong the effect of an agent, it is desirable to slow the absorption of the agent from subcutaneous or intramuscular injection. This may be accomplished by the use of a liquid suspension of crystalline or amorphous material having poor water solubility. The rate of absorption of the agent then depends upon its rate of dissolution, which, in turn, may depend upon crystal size and crystalline form. Alternatively, delayed absorption of a parenterally administered agent is accomplished by dissolving or suspending the agent in an oil vehicle.

Injectable depot forms are made by forming microencapsule matrices of the subject compounds in biodegradable polymers such as polylactide-polyglycolide. Depending on the ratio of agent to polymer, and the nature of the particular polymer employed, the rate of agent release can be controlled. Examples of other biodegradable polymers include poly(orthoesters) and poly(anhydrides). Depot injectable formulations are also prepared by entrapping the agent in liposomes or microemulsions that are compatible with body tissue.

When the compounds of the present invention are administered as pharmaceuticals, to humans and animals, they can be given per se or as a pharmaceutical composition containing, for example, 0.1 to 99.5% (more preferably, 0.5 to 90%) of active ingredient in combination with a pharmaceutically acceptable carrier.

Apart from the above-described compositions, use may be made of covers, e.g., plasters, bandages, dressings, gauze pads and the like, containing an appropriate amount of a therapeutic. As described in detail above, therapeutic compositions may be administered/delivered on stems, devices, prosthetics, and implants.

The tissue sample for analysis is typically blood, plasma, serum, mucous fluid or cerebrospinal fluid from the patient. The sample is analyzed, for example, for levels or profiles of antibodies to RAGE peptide, e.g., levels or profiles of humanized antibodies. ELISA methods of detecting antibodies specific to RAGE are described in the Examples.

The antibody profile following passive immunization typically shows an immediate peak in antibody concentration followed by an exponential decay. Without a further dosage, the decay approaches pretreatment levels within a period of days to months depending on the half-life of the antibody administered.

In some methods, a baseline measurement of antibody to RAGE in the patient is made before administration, a second measurement is made soon thereafter to determine the peak antibody level, and one or more further measurements are made at intervals to monitor decay of antibody levels. When the level of antibody has declined to baseline or a predetermined percentage of the peak less baseline (e.g., 50%, 25% or 10%), administration of a further dosage of antibody is administered. In some methods, peak or subsequent measured levels less background are compared with reference levels previously determined to constitute a beneficial prophylactic or therapeutic treatment regime in other patients. If the measured antibody level is significantly less than a reference level (e.g., less than the mean minus one standard deviation of the reference value in population of patients benefiting from treatment) administration of an additional dosage of antibody is indicated.

### EXAMPLES

The invention now being generally described, it will be more readily understood by reference to the following examples, which are included merely for purposes of illustration of certain aspects and embodiments of the present invention, and are not intended to limit the invention.

### Example 1

### Preparation of RAGE Constructs

The amino acid sequences of murine RAGE (mRAGE, Genbank accession no. NP_{—}031451; SEQ ID NO: 3) and human RAGE (hRAGE, Genbank accession no. NP_{—}00127.1; SEQ ID NO: 1) are shown in FIG. 1A-1C. Full length cDNAs encoding mRAGE (accession no. NM_{—}007425.1; SEQ ID NO: 4) and hRAGE (accession no. NM_{—}001136; SEQ ID NO: 2) were inserted into the Adori1-2 expression vector, which comprises a cytomegalovirus (CMV) promoter driving expression of the cDNA sequences, and contains adenovirus elements for virus generation. A human RAGE-Fc fusion protein formed by appending amino acids 1-344 of human RAGE to the Fc domain of human IgG was prepared by expressing a DNA construct encoding the fusion protein in cultured cells using the Adori expression vector. A human RAGE V-region-Fc fusion protein formed by appending amino acids 1-118 of human RAGE to the Fc domain of human IgG was similarly prepared. Human and murine RAGE-strep tag fusion proteins formed by appending a streptavidin (strep) tag sequence (WSHPQFEK) (SEQ ID NO: 5) to amino acids 1-344 of human or murine RAGE, respectively, were prepared by expressing DNA constructs encoding the RAGE-strep tag fusion proteins, also using Adori expression vectors. All constructs were verified by extensive restriction digest analyses and by sequence analyses of cDNA inserts within the plasmids

Recombinant adenovirus (Ad5 E1a/E3 deleted) expressing the full-length RAGE, hRAGE-Fc, and hRAGE V-domain-Fc were generated by homologous recombination in a human embryonic kidney cell line 293 (HEK293) (ATCC, Rockland Md.). Recombinant adenovirus virus was isolated and subsequently amplified in HEK293 cells. The virus was released from infected HEK293 cells by three cycles of freeze thawing. The virus was further purified by two cesium chloride centrifugation gradients and dialyzed against phosphate buffered saline (PBS) pH 7.2 at 4° C. Following dialysis, glycerol was added to a concentration of 10% and the virus was stored at −80° C. until use. Viral constructs were characterized for infectivity (plaque forming units on 293 cells), PCR analysis of the virus, sequence analysis of the coding region, expression of the transgene, and endotoxin measurements.

Adori expression vectors containing DNA encoding human RAGE-Fc, human RAGE-V region-Fc, and human and murine RAGE-strep tag fusion proteins were stably transfected into Chinese Hamster Ovary (CHO) cells using lipofectin (Invitrogen). Stable transfectants were selected in 20 nM and 50 nM methotrexate. Conditioned media were harvested from individual clones and analyzed with the use of sodium dodecyl sulfate-polyacrylamide gel electrophoresis (SDS-PAGE) and Western blotting to confirm RAGE expression. (Kaufman, R. J., 1990, Methods in Enzymology, 185:537-66; Kaufman, R. J., 1990, Methods in Enzymology, 185:487-511; Pittman, D. D. et al., 1993, Methods in Enzymology, 222: 236-237).

CHO or transduced HEK 293 cells expressing soluble RAGE fusion proteins were cultured to harvest conditioned medium for protein purification. Proteins were purified with the use of indicated affinity-tag methods. Purified proteins were subjected to reducing and non-reducing SDS-PAGE, visualized by Coomassie Blue staining (Current Protocols in Protein Sciences, Wiley Interscience), and shown to be of the expected molecular weights.

### Example 2

### Generation of Murine Anti-RAGE Monoclonal Antibodies

6-8 week old female BALB/c mice (Charles River, Andover, Mass.) were immunized subcutaneously with the use of a GeneGun device (BioRad, Hercules, Calif.). The pAdori expression vector containing cDNA encoding full-length human RAGE was pre-absorbed onto colloidal gold particles (BioRad, Hercules, Calif.) before subcutaneous administration. Mice were immunized with 3 ug of vector twice per week, for two weeks. Mice were bled one week after the last immunization and antibody titers were evaluated. The mouse with highest RAGE-antibody titer received one additional injection of 10 μg of recombinant human RAGE-strep protein three days before cell fusion.

Splenocytes were fused with mouse myeloma cells P3X63Ag8.653 (ATCC, Rockville, Md.) at a 4:1 ratio using 50% polyethylene glycol (MW 1500) (Roche Diagnostics Corp, Mannheim, Germany). After fusion, cells were seeded and cultured in 96-well plates at 1×10⁵ cells/well in the RPM11640 selection medium, containing 20% FBS, 5% Origen (IGEN International Inc. Gaithersburg, Md.), 2 mM L-glutamine, 100 U/ml penicillin, 100 μg/ml streptomycin, 10 mM HEPES and 1× hypoxanthine-aminopterin-thymidine (Sigma, St. Louis, Mo.).

### Example 3

### Generation of Rat Anti-RAGE Monoclonal Antibodies

LOU rats (Harlan, Harlan, Mass.) rats were immunized subcutaneously with the use of a GeneGun (BioRad, Hercules, Calif.). The pAdori expression vector containing cDNA encoding full-length murine RAGE was pre-absorbed onto colloidal gold particles (BioRad, Hercules, Calif.) before subcutaneous administration. Rats were immunized with 3 ug of vector once every two weeks for four times. Rats were bled one week after the last immunization and antibody titers were evaluated. The rat with highest RAGE-antibody titer received one additional injection of 10 μg of recombinant murine RAGE-strep protein three days before cell fusion.

Splenocytes were fused with mouse myeloma cells P3X63Ag8.653 (ATCC, Rockville, Md.) at a 4:1 ratio using 50% polyethylene glycol (MW 1500) (Roche Diagnostics Corp, Mannheim, Germany). After fusion, cells were seeded and cultured in 96-well plates at 1×10⁵ cells/well in the RPMI1640 selection medium, containing 20% FBS, 5% Origen (IGEN International Inc. Gaithersburg Md.), 2 mM L-glutamine, 100 U/ml penicillin, 100 μg/ml streptomycin, 10 mM HEPES and 1× hypoxanthine-aminopterin-thymidine (Sigma, St. Louis, Mo.).

### Example 4

### Hybridoma Screening

Panels of rat anti-murine RAGE and murine anti-human RAGE mAbs were generated by cDNA immunization using the GeneGun, and the Adori expression plasmids expressing the full-length coding region of murine or human RAGE. Hybridoma supernatants were screened for binding to recombinant human or murine RAGE-Fc by ELISA and by FACS analysis on human embryonic kidney cells (HEK-293) transiently expressing RAGE. Positive supernatants were further tested for their ability to neutralize RAGE binding to the ligand HMGB1. Seven rat monoclonal antibodies (XT-M series) and seven mouse monoclonal antibodies (XT-H series) were identified. Selected hybridomas were subcloned four times by serial dilution and once by FACS sorting. Conditioned media were harvested from the stable hybridoma cultures and immunoglobulins were purified using Protein A antibody purification columns (Millipore Billerica, Mass.). The Ig class of each mAb was determined with a mouse mAb isotyping kit or rat mAb isotyping kit as indicated (IsoStrip; Boehringer Mannheim Corp.). The isotypes of the selected rat and mouse monoclonal antibodies are set forth in Table 1 (below).

TABLE 1

| Rat monoclonal | | | Murine monoclonal | | |
|---|---|---|---|---|---|
| anti-muRAGE antibodies | | | anti-huRAGE antibodies | | |
| Hybridoma | | | Hybridoma | | |
| clones | Mabs | Ig isotypes | clones | Mabs | Ig isotypes |
| 1mRAGEP3/1* | XT-M1 | Rat lgG2a, k | 1hRAGEP3/6* | XT-H1 | Mouse lgG1, k |
| 1mRAGEP3/7 | XT-M2 | Rat lgG2b, k | 1hRAGEP3/16* | XT-H2 | Mouse lgG1, k |
| 1mRAGEP3/8 | XT-M3 | Rat lgG2a, k | 1hRAGEP3/18 | XT-H3 | Mouse lgG1, k |
| 1mRAGEP3/10* | XT-M4 | Rat lgG2b, k | 1hRAGEP3/48 | XT-H4 | Mouse lgG1, k |
| 1mRAGEP3/15 | XT-M5 | Rat lgG2a, k | 1hRAGEP3/55* | XT-H5 | Mouse lgG1, k |
| 1mRAGEP3/16 | XT-M6 | Rat lgG2b, k | 1hRAGEP3/65 | XT-H6 | Mouse lgG1, k |
| 1mRAGEP3/18* | XT-M7 | Rat lgG2b, k | 1hRAGEP3/66 | XT-H7 | Mouse lgG1, k |

### Example 5

### FACS Analysis

Human 293 cells were infected with the human and murine RAGE adenovirus. Infected cells were suspended in PBS containing 1% BSA at a density of 4×10⁴ cells/ml. Cells were incubated with 100 ul of sample (diluted immune sera, hybridoma supernatants or purified antibodies) for 30 min at 4° C. After washing, cells were incubated with PE-labeled goat, anti-mouse, IgG, F(ab′)₂ (DAKO Corporation GlostrupDenmark) for 30 min at 4° C. in the dark. Cell-associated fluorescence signals were measured by a FACScan flow cytofluorometer (Becton Dickinson) using 5000 cells per treatment. Propidium iodide was used to identify dead cells, which were excluded from the analysis. The seven murine monoclonal antibodies XT-H1 to XT-H7 and the seven rat monoclonal antibodies XT-M1 to XT-M7 were shown by FACS analysis to bind to cell-surface hRAGE (Table 2).

### Example 6

### ELISA Binding Assay

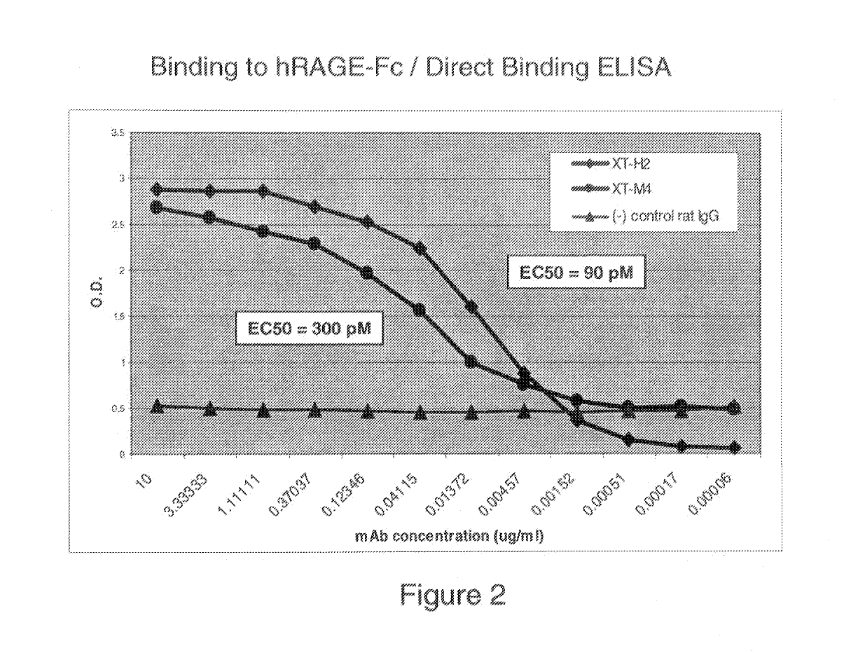

Antibodies were purified from hybridoma supernatants using standard procedures. Purified antibodies were evaluated for binding to soluble forms of RAGE with the use of ELISA. Ninety-six well plates (Corning, Corning, N.Y.) were coated with 100 ul of recombinant human RAGE-Fc or recombinant human RAGE V-region-Fc (1 μg/ml) and incubated overnight at 4° C. After washing and blocking with PBS containing 1% BSA and 0.05% Tween-20, 100 ul of sample (samples were in several forms: diluted immune serum, hybridoma supernatants, or purified antibodies, as indicated) was added and incubated for 1 hour at room temperature. The plates were washed with PBS, pH 7.2 and bound anti-RAGE antibodies were detected with the use of peroxidase-conjugated goat, anti-mouse IgG (H+L) (IgG) (Pierce, Rockford, Ill.) followed by incubation with the substrate TMB (BioFX Laboratories Owings Mills, Md. Laboratories). Absorbance values were determined at 450 nm in a spectrophotometer. The concentrations of monoclonal antibodies were determined with the use of peroxidase-labeled goat, anti-mouse IgG (Fcγ) (Pierce Rockford, Ill.) and a standard curve was generated by a purified, isotype-matched mouse IgG. ELISA results for the abilities of the seven murine antibodies XT-H1 to XT-H7 and the seven rat antibodies XT-M1 to XT-M7 to bind to hRAGE-Fc, hRAGE V-region-Fc, mRAGE-Fc, and mRAGE-strep, are summarized in Table 2. As shown in FIGS. 2 and 3, rat antibody XT-M4 and murine antibody XT-H2 both bind to human RAGE-Fc and to the V-domain of hRAGE. The EC50 values for binding of XT-M4 to human RAGE and to human RAGE V-domain were 300 pM and 100 pM, respectively. The EC50 values for binding of XT-H2 to human RAGE and human RAGE V-domain were 90 pM and 100 pM, respectively.

TABLE 2

| | | | | | ELISA | ELISA |
|---|---|---|---|---|---|---|
| | FACS | FACS | ELISA | ELISA | mRAGE- | hRAGE- |
| Mabs | hRAGE-Fc | mRAGE-Fc | hRAGE-Fc | mRAGE-Fc | strep | V-Fc (CM) |
| XT-H1 | + | + | +++ | − | + | − |
| XT-H2 | + | − | +++ | − | − | ++ |
| XT-H3 | + | − | +++ | | − | |
| XT-H4 | + | − | +++ | | − | |
| XT-H5 | + | − | +++ | − | − | ++ |
| XT-H6 | + | − | +++ | − | − | |
| XT-H7 | + | − | +++ | − | +/− | |
| XT-M1 | − | + | − | +++ | +++ | +++ |
| XT-M2 | + | + | ++ | + | + | + |
| XT-M3 | + | + | − | | | |
| XT-M4 | + | + | ++ | + | + | + |
| XT-M5 | − | + | − | | | |
| XT-M6 | + | + | ++ | + | + | + |
| XT-M7 | + | + | ++ | +++ | +++ | +++ |

### Example 7

### RAGE Ligand and Antibody Competition ELISA Binding Assays

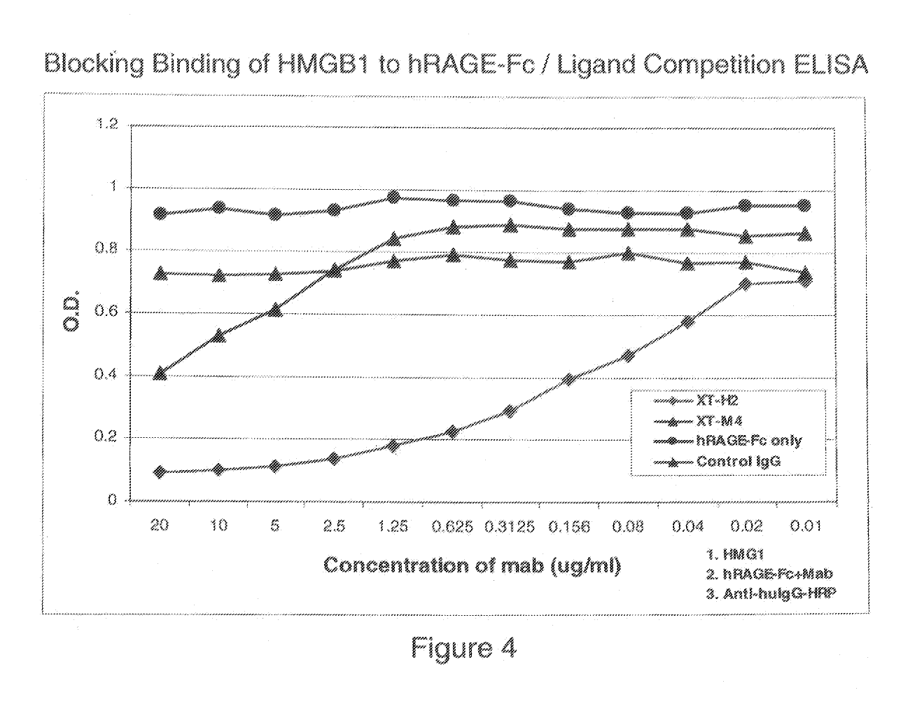

To determine whether RAGE monoclonal antibodies affect the binding of a RAGE ligand (HMGB1; Sigma, St. Louis, Mo.) to RAGE, competition ELISA binding assays were performed. Ninety-six well plates were coated with 1 μg/ml of HMGB1 overnight at 4° C. Wells were washed and blocked as described above and exposed to 100 μl of pre-incubated mixtures of RAGE-Fc or TrkB-Fc (a non-specific Fc control), at 0.1 μg/ml, plus various forms of the indicated antibody preparation (dilutions of immune sera, hybridoma supernatants or purified antibodies) for 1 hour at room temperature. Plates were washed with PBS, pH 7.2 and ligand-bound recombinant human RAGE-Fc was detected with the use of peroxidase-conjugated goat, anti-human IgG (Fcγ) (Pierce, Rockford, Ill.), followed by incubation with the substrate TMB (BioFX Laboratories Owings Mills, Md. Laboratories Owings Mills, Md.). Binding of recombinant human RAGE-Fc to ligand without any antibodies or with diluted pre-immune serum was used as a control and defined as 100% binding. The abilities of the seven murine antibodies XT-H1 to XT-H7 and the seven rat antibodies XT-M1 to XT-M7 to block the binding of HMGB1 to hRAGE-Fc as determined by the competition ELISA binding assay are shown in Table 3. Table 3 also summarizes the abilities of murine antibodies XT-H1, XT-H2, and XT-H5 to block the binding to RAGE of a different ligand of hRAGE, amyloid β 1-42 peptide, and the abilities of rat antibodies XT-M1 to XT-M7 to block the binding of HMGB1 to murine RAGE-Fc, as determined by similar competition ELISA binding assays. As shown in FIG. 4, rat antibody XT-M4 and murine antibody XT-H2 both block the binding of HMGB1 to human RAGE.

TABLE 3

| | RAGE ligand competition ELISA | | | Antibody compe- |
|---|---|---|---|---|
| | binding assays | | | tition ELISA |
| | | hRAGE-Fc + | | binding assays |
| | hRAGE-Fc + | Aβ 1-42 | mRAGE-Fc + | ELISA hRAGE- |
| Mabs | HMGB1 | peptide | HMGB1 | V-Fc (CM) |
| XT-H1 | − | + | | |
| XT-H2 | + | +++ | | |
| XT-H3 | − | | | |
| XT-H4 | +/− | | | |
| XT-H5 | + | +++ | | |
| XT-H6 | − | | | |
| XT-H7 | +/− | | | |
| XT-M1 | − | | − | − |
| XT-M2 | + | | + | XT-H3 & XT-H7 |
| | | | | compete |
| XT-M3 | − | | − | |
| XT-M4 | ++ | | + | XT-H2 & XT-H7 |
| | | | | compete |
| XT-M5 | − | | − | |
| XT-M6 | + | | + | − |
| XT-M7 | + | | + | − |

A similar competition approach was used to determine the relative binding epitopes between pairs of antibodies. First, 1 μg/ml of recombinant human RAGE-Fc was coated on ninety six-well plates over night at 4° C. After washing and blocking (see above) wells were exposed to 100 μl of pre-incubated mixtures of biotinylated target antibody and dilutions of a competing antibody for 1 hour at room temperature. Bound biotinylated antibody was detected using peroxidase-conjugated streptavidin (Pierce, A similar competition approach was used to determine the relative binding epitopes between pairs of antibodies. First, 1 μg/ml of recombinant human RAGE-Fc was coated on ninety six-well plates over night at 4° C. After washing and blocking (see above) wells were exposed to 100 μl of pre-incubated mixtures of biotinylated target antibody and dilutions of a competing antibody for 1 hour at room temperature. Bound biotinylated antibody was detected using peroxidase-conjugated streptavidin (Pierce, Rockford, Ill.) followed by incubation with the substrate TMB (BioFX Laboratories Owings Mills, Md. Laboratories). Binding of biotinylated antibody to recombinant human RAGE-Fc without any competing antibodies was used as a control and defined as 100%. Results of competition ELISA binding assays analyzing the competition between rat and murine antibodies for binding to hRAGE are shown in Table 3. FIG. 5 present a graph of data from competition ELISA binding assays analyzing the competition between rat XT-M4 and antibodies XT-H1, XT-H2, XT-H5, XT-M2, XT-M4, XT-M6, and XT-M7 for binding to hRAGE. The competition ELISA binding data shown in FIG. 5 demonstrate that XT-M4 and XT-H2 bind to overlapping sites on human RAGE.

### Example 8

### BIACORE™ Binding Assays of Binding of Murine and Rat Anti-RAGE Antibodies to Human and Murine RAGE-Fc

A. Binding to Human and Murine RAGE

The binding of selected murine and rat anti-RAGE antibodies to human and murine RAGE and to the V domains of human and murine RAGE was analyzed by BIACORE® direct binding assay. Assays were performed using human or murine RAGE-Fc coated on a CM5 chip at high density (2000 RU) using standard amine coupling. Solution of the anti-RAGE antibodies at two concentrations, 50 and 100 nm, were run over the immobilized RAGE-Fc proteins in duplicate. BIACORE™ technology utilizes changes in the refractive index at the surface layer upon binding of the anti-RAGE antibodies to the immobilized RAGE antigen. Binding is detected by surface plasmon resonance (SPR) of laser light refracting from the surface. Results of the BIACORE™ direct binding assays are summarized in Table 4.

TABLE 4

| Rat anti-muRAGE | | | Murine anti-huRAGE | | |
|---|---|---|---|---|---|
| antibodies | | | antibodies | | |
| | huRAGE- | muRAGE- | | huRAGE- | muRAGE- |
| Mabs | Fc | Fc | Mabs | Fc | Fc |
| XT-M1 | | +++ | XT-H1 | +++ | +/− |
| XT-M2 | + | ++ | XT-H2 | +++ | − |
| XT-M3 | | | XT-H3 | + | + |
| XT-M4 | +++ | +++ | XT-H4 | + | + |
| XT-M5 | | | XT-H5 | ++ | − |
| XT-M6 | ++ | +++ | XT-H6 | +++ | − |
| XT-M7 | ++ | +++ | XT-H7 | +++ | − |

The kinetic rate constants (kₐ and k_{d}) and association and dissociation constants (Kₐ and K_{d}) for the binding of murine and rat anti-RAGE antibodies to human and murine RAGE were determined by BIACORE™ direct binding assay. Analysis of the signal kinetics data for on-rate and off-rate allows the discrimination between non-specific and specific interactions. Kinetic rate constants and equilibrium constants determined by the BIACORE™ direct binding assay for the binding of murine XT-H2 antibody and rat XT-M4 antibody to hRAGE-Fc are shown in Table 5.

TABLE 5
Kinetic rate constants and equilibrium constants for binding to hRAGE-Fc

| | kₐ (1/Ms) | k_{d} (1/s) | Kₐ (1/M) | K_{d} (M) | R_{Max} | X² |
|---|---|---|---|---|---|---|
| XT-H2 | 5.76 × 10⁶ | 5.04 × 10⁻⁴ | 1.14 × 10¹⁰ | 8.76 × 10⁻¹¹ | 55.7 | 2.68 |
| XT-M4 | 1.16 × 10⁶ | 1.16 × 10⁻³ | 1.00 × 10⁹ | 9.95 × 10⁻¹⁰ | 89.9 | 14.3 |

B. Binding to the Human RAGE V-Domain

The kinetic rate constants and association and dissociation constants for the binding of murine and rat anti-RAGE antibodies to the human RAGE V-domain were also determined by BIACORE™ direct binding assay. Human RAGE V-domain-Fc was captured by anti-human Fc antibodies coated on a CM5 chip, and BIACORE™ direct binding assays of the binding of murine and rat anti-RAGE antibodies to the immobilized hRAGE V domain-Fc were performed as described above for assays of binding to full-length RAGE-Fc.

### Example 9

### Amino Acid Sequences of Anti-RAGE Antibody Variable Regions

DNA sequences encoding the light and heavy chain variable regions of murine anti-RAGE antibodies XT-H1, XT-H2, XT-H3, XT-H5 and XT-H7, and of rat anti-RAGE antibody XT-M4 were cloned and sequenced, and the amino acid sequences of the variable regions were determined. The aligned amino acid sequences of the heavy chain variable regions of these six antibodies are shown in FIG. 6, and the aligned amino acid sequences of the light chain variable regions are shown in FIG. 7.

### Example 10

### Isolation of Rabbit, Baboon, and Cynomologus Monkey cDNA Sequences Encoding RAGE

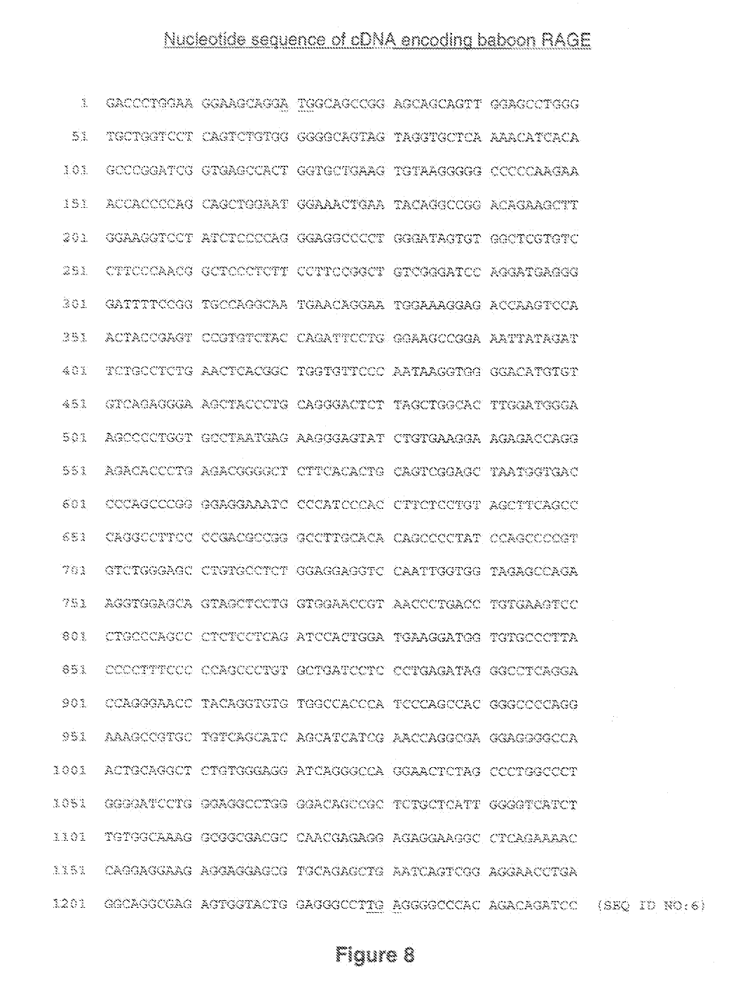

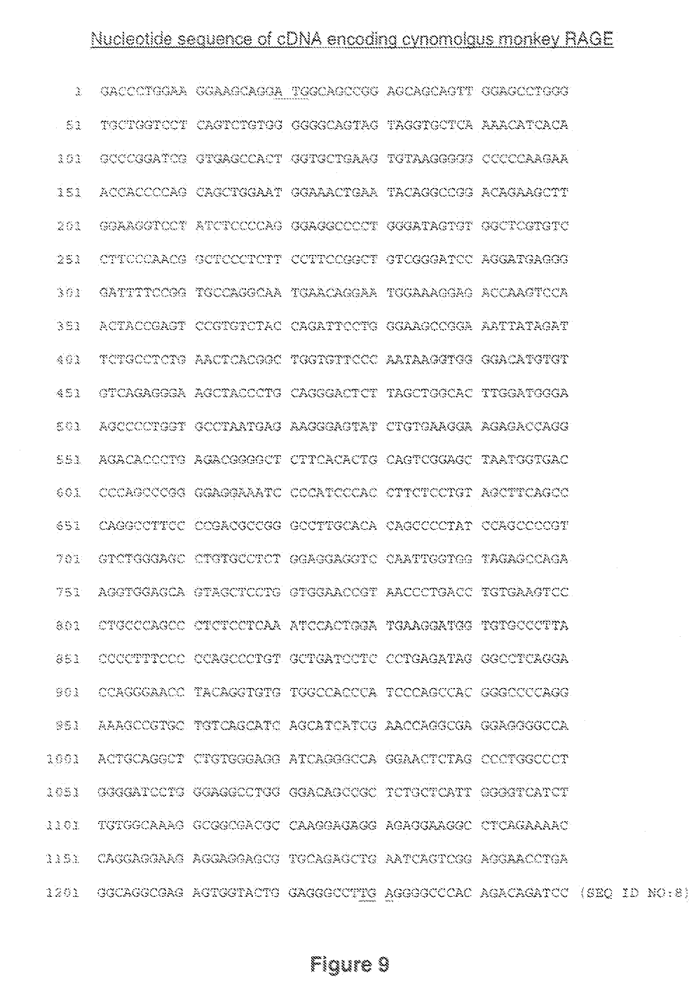

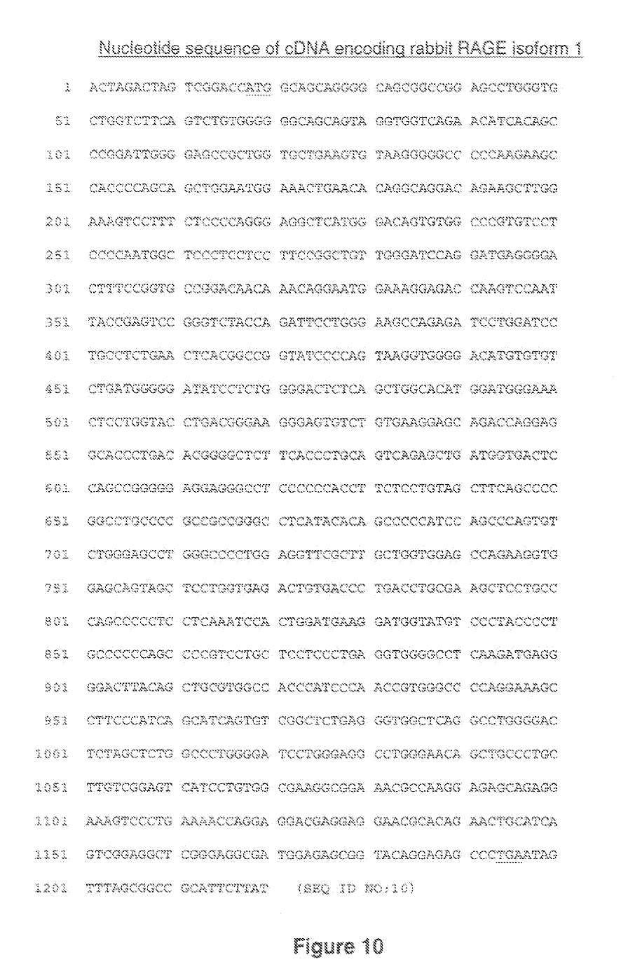

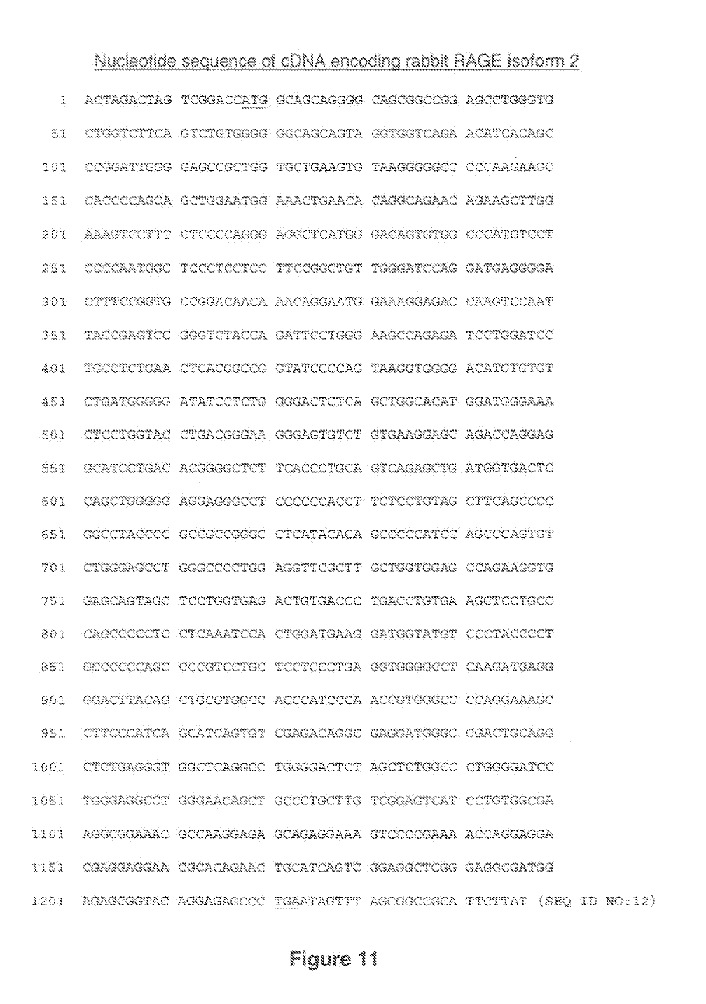

cDNA sequences encoding RAGE were isolated and cloned using standard reverse transcription-polymerase chain reaction (RT-PCR) methods. RNA was extracted and purified from lung tissue using Trizol (Gibco Invitrogen, Carlsbad, Calif.) via the manufacturer's protocol. mRNA was reverse transcribed to generate cDNA using TaqMan Reverse Transcription Reagent (Roche Applied Science Indianapolis, Ind.) and manufacturer's protocol. Cynomologus monkey (Macaca fascicularis) and baboon (Papio cyanocephalus) RAGE sequences were amplified from cDNA using Invitrogen Taq DNA polymerase (Invitrogen, Carlsbad Calif.) and protocol and oligonucleotides (5′-GACCCTGGAAGGAAGCAGGATG (SEQ ID NO: 59) and 5′-GGATCTGTCTGTGGGCCCCTCAAGGCC) (SEQ ID NO: 60) that add SpeI and EcoRV restriction sites. PCR amplification products were digested with SpeI/EcoRV and cloned into the corresponding sites in the plasmid pAdori1-3. Rabbit RAGE was cloned using RT-PCR as described above using the oligonucleotides: 5′-ACTAGACTAGTCGGACCATGGCAGCAGGGGCAGCGGCCGGA (SEQ ID NO: 61) and 5′-ATAAGAATGCGGCCGCTAAACTATTCAGGGCTCTCCTGTACCGCTCTC (SEQ ID NO: 62) that add SpeI and NotI sites, and cloned into the corresponding sites in pAdori1-3. The nucleotide sequences of the cloned cDNA sequences encoding baboon, monkey, and two isoforms of rabbit RAGE in the resultant plasmids were determined. The nucleotide sequence encoding baboon RAGE is shown in FIG. 8 (SEQ ID NO: 6), and the nucleotide sequence encoding cynomologus monkey RAGE is shown in FIG. 9 (SEQ ID NO: 8). The nucleotide sequences encoding two isoforms of rabbit RAGE are shown in FIG. 10 (SEQ ID NO:10) and FIG. 11 (SEQ ID NO:12).

### Example 11

### Isolation of a Genomic DNA Sequence Encoding Baboon RAGE

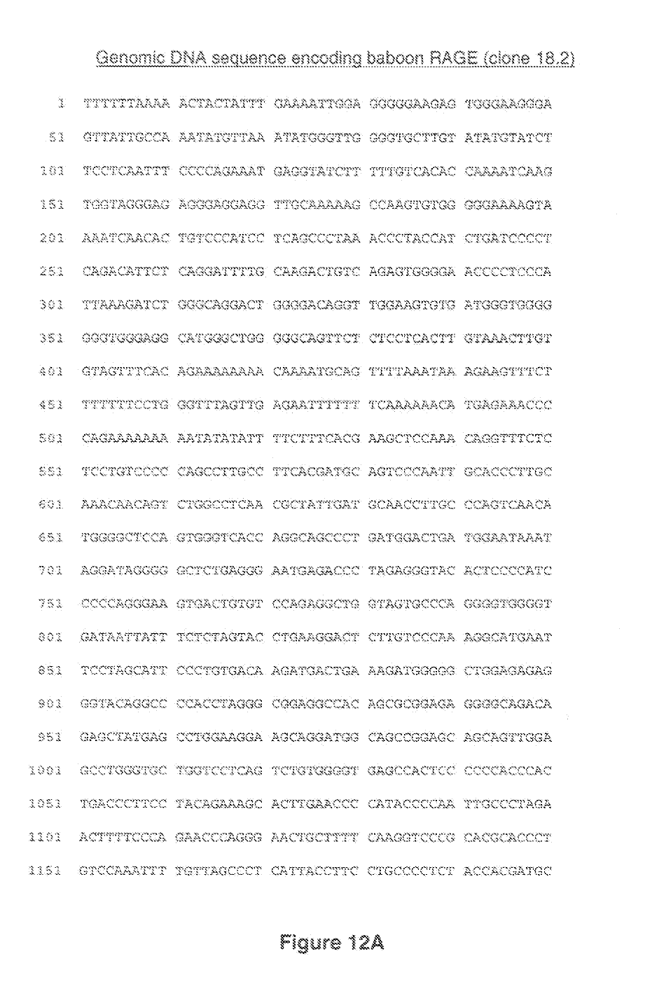

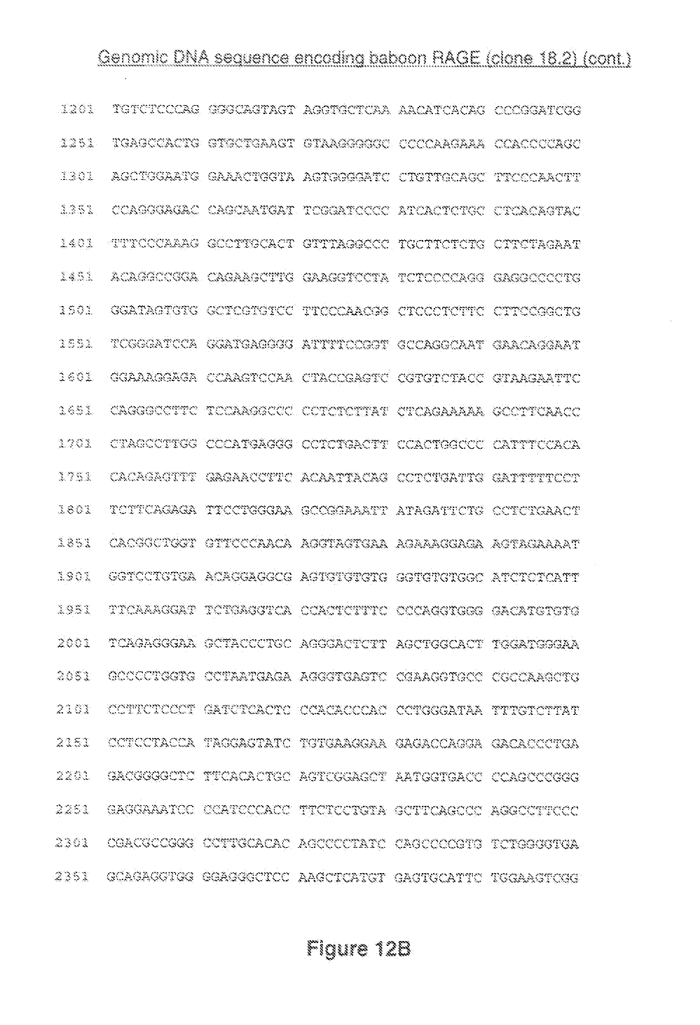

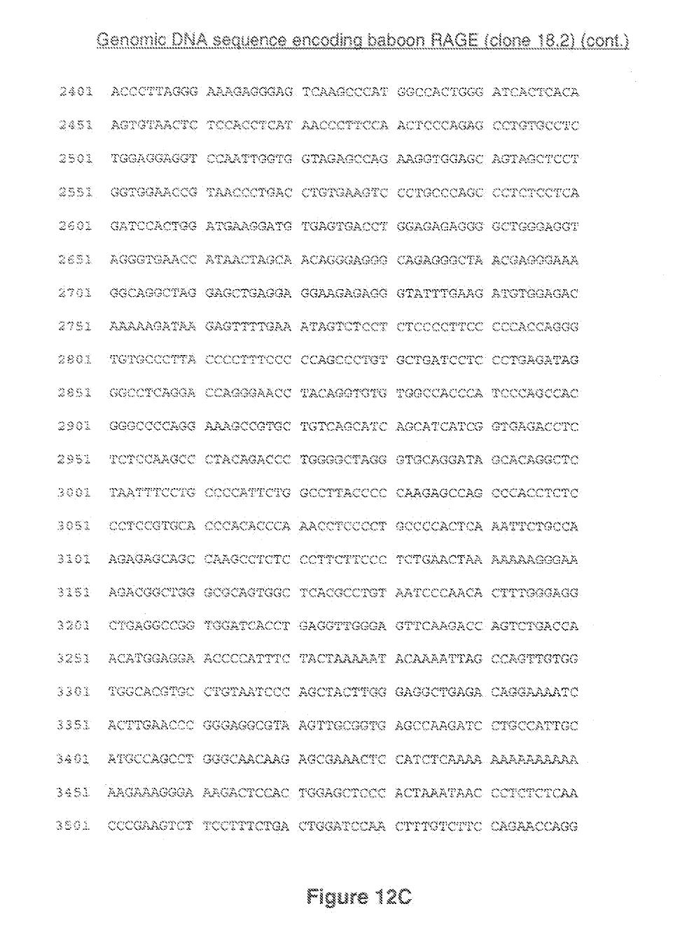

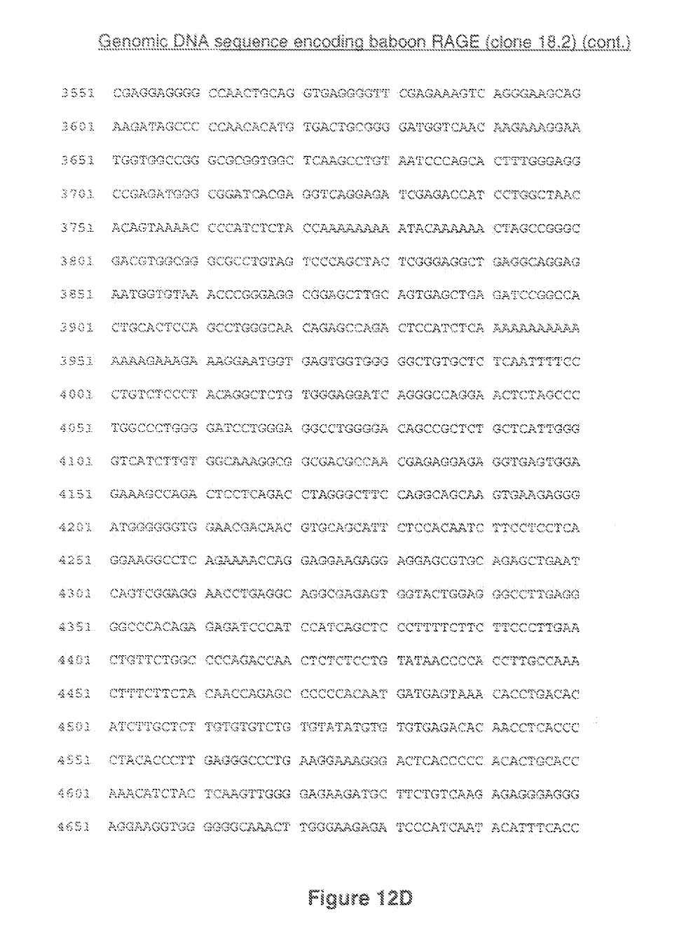

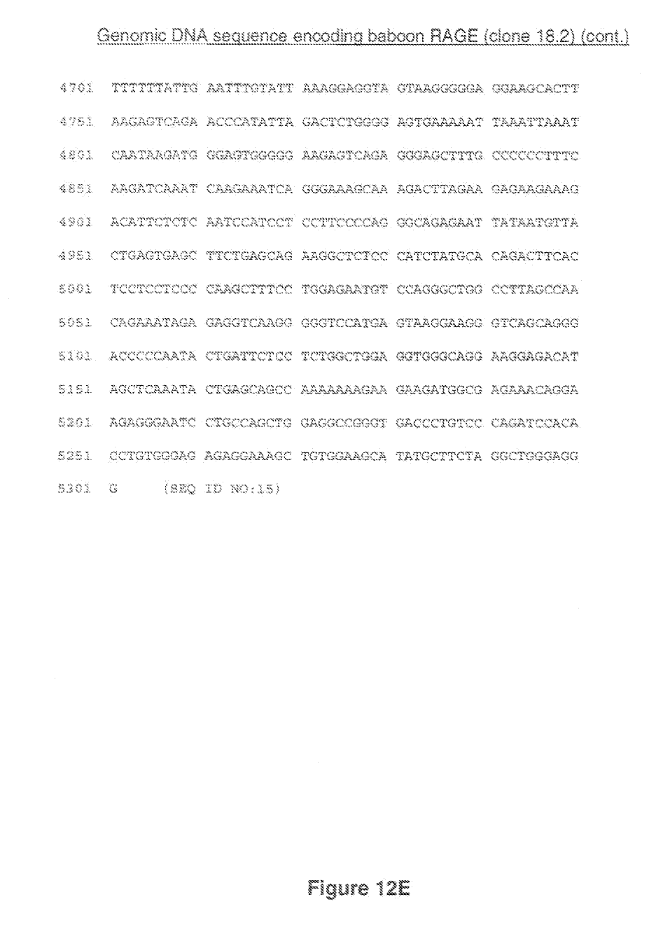

A baboon genomic DNA sequence encoding RAGE was isolated using standard genomic cloning techniques (e.g., see Sambrook, J. et al., Molecular Cloning: A Laboratory Manual, 2^{nd} Ed., 1989, Cold Spring Harbor Laboratory Press, Cold Spring Harbor, N.Y.). A baboon (Papio cyanocephalus) Lambda genomic library (Stratagene, La Jolla, C) in the Lambda DASH II vector was screened using ³²P random primed human RAGE cDNA. Positive phage plaques were isolated and subjected to two additional rounds of screening to obtain single isolates. Lambda DNA was prepared, digested with NotI, and size fractionated to separate insert DNA from Lambda genomic arms, using common procedure. The NotI fragments were ligated into NotI-digested pBluescript SK+, and the insert was sequenced using RAGE specific primers. The clone that was obtained was designated clone 18.2. The nucleotide sequence of the cloned baboon genomic DNA encoding a baboon RAGE is shown in FIGS. 12A-12-E (SEQ ID NO: 15).

### Example 12

### Chimeric XT-M4 Antibody

A chimeric XT-M4 was generated by fusing the light and heavy chain variable regions of rat anti-murine RAGE antibody XT-M4 to human kappa light chain and IgG1 heavy chain constant regions, respectively. To reduce the potential Fc-mediated effector activity of the antibody, chimeric mutations L234A and G237A were introduced into XT-M4 in the human IgG1 Fc region. The chimeric antibody is given molecule number XT-M4-A-1. The chimeric XT-M4 antibody contains 93.83% human amino acid sequence, and 6.18% rat amino acid sequence.

### Example 13

### Assessing the Binding of Chimeric XT-M4 to RAGE

The abilities of chimeric antibody XT-M4 and selected rat and murine anti-RAGE antibodies to bind to human RAGE and RAGE of other species, and to block the binding of RAGE ligands was measured by ELISA and BIACORE™ binding assays.

A. Binding to Soluble Human RAGE Measured by BIACORE™ Binding Assay

The binding of chimeric antibody XT-M4, the parental rat antibody XT-M4, and murine antibodies XT-H2 and XT-H5 to soluble human RAGE (hRAGE-SA) was measured by BIACORE™ capture binding assay. The assays were performed by coating antibodies onto a CM5 BIA chip with 5000-7000 RU. Solutions of a purified soluble human streptavidin-tagged RAGE (hRAGE-SA) at concentrations of 100 nM, 50 nM, 25 nM, 12.5 nM, 6.25 nM, 3.12 nM, 1.56 nM and 0 nM were flowed over the immobilized antibodies in triplicate, and kinetic rate constants (kₐ and k_{d}) and association and dissociation constants (Kₐ and K_{d}) for binding to hRAGE-SA were determined. The results are shown in Table 6.

TABLE 6
Kinetic rate constants and equilibrium constants for binding to hRAGE-SA

| | kₐ (1/Ms) | k_{d} (1/s) | Kₐ (1/M) | K_{d} (M) | R_{Max} | X² |
|---|---|---|---|---|---|---|
| XT-M4 | 3.78 × 10⁶ | 1.86 × 10⁻² | 2.03 × 10⁸ | 4.92 × 10⁻⁹ | 61.5 | 0.563 |
| chimeric | 4.39 × 10⁶ | 2.48 × 10⁻² | 1.77 × 10⁸ | 5.66 × 10⁻⁹ | 33.1 | 0.436 |
| antibody XT-M4 | | | | | | |
| XT-H2 | 1.10 × 10⁶ | 1.16 × 10⁻³ | 9.48 × 10⁸ | 1.06 × 10⁻⁹ | 48.1 | 2.7 |
| XT-H5 | 1.66 × 10⁶ | 4.51 × 10⁻³ | 3.69 × 10⁸ | 2.71 × 10⁻⁹ | 24.5 | 0.996 |

The XT-M4 antibody and chimeric antibody XT-M4 bind to monomeric soluble human RAGE with similar kinetics. The affinity of chimeric XT-M4 for human soluble monomeric RAGE is approximately 5.5 nM.
B. RAGE Ligand Competition ELISA Binding Assay

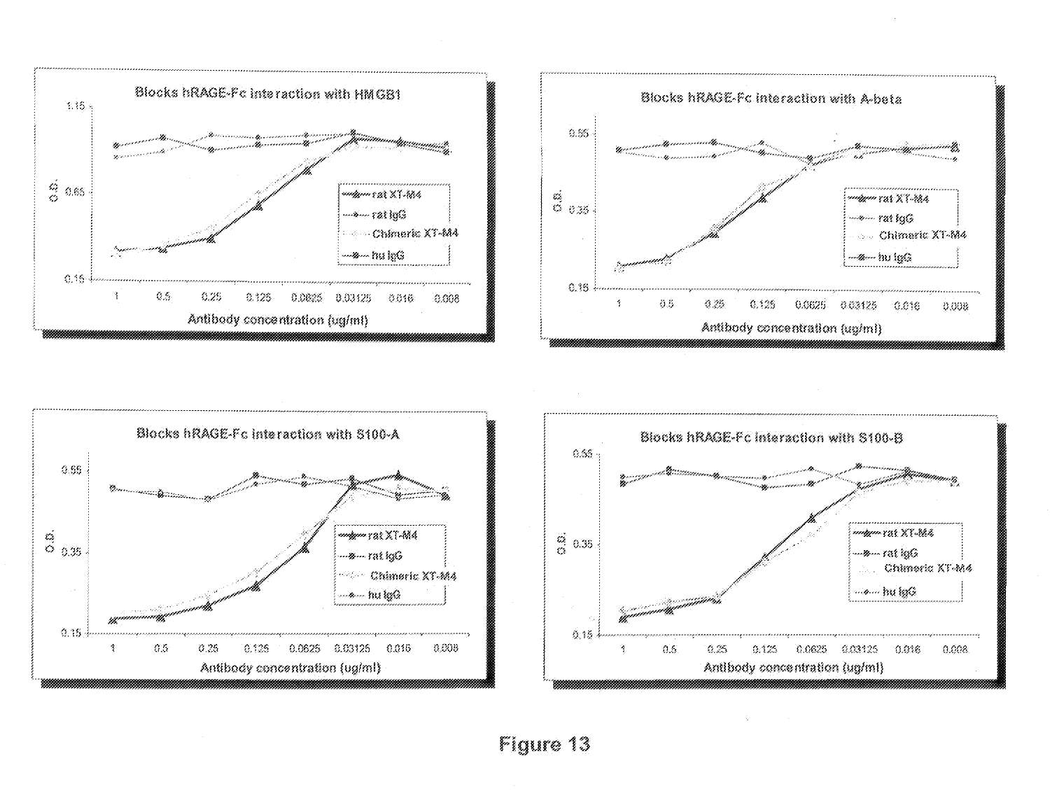

The abilities of chimeric antibody XT-M4 antibody and rat antibody XT-M4 to block the binding of RAGE ligands HMGB1, amyloid β 1-42 peptide, S100-A, and S100-B to hRAGE-Fc were determined by ligand competition ELISA binding assay as described in Example 7. As shown in FIG. 13, chimeric antibody XT-M4 and XT-M4 are nearly identical in their abilities to block the binding of HMGB1, amyloid β 1-42 peptide, S100-A, and S100-B to human RAGE.

C. Antibody Competition ELISA Binding Assay

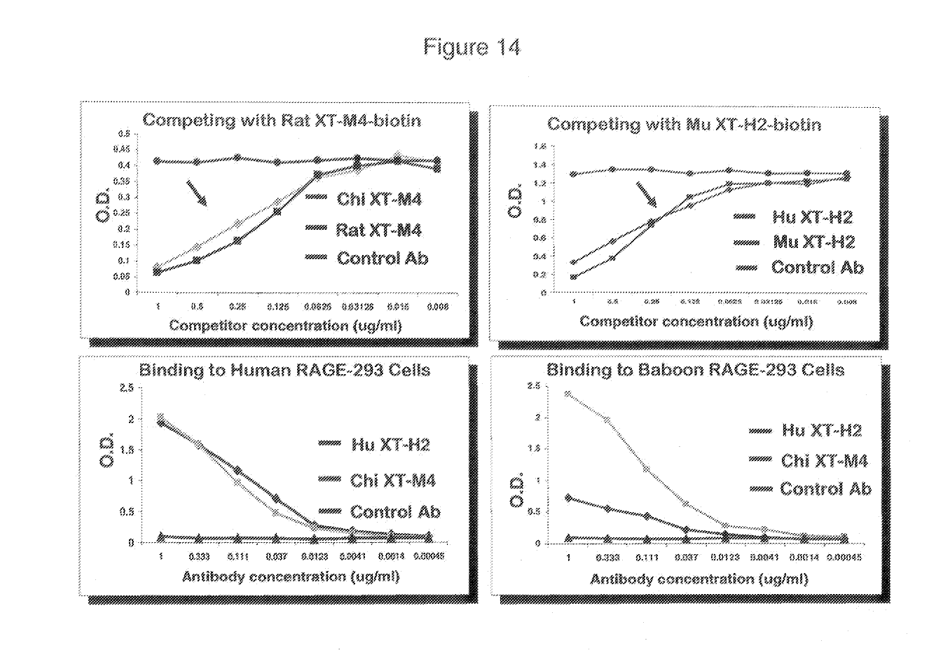

The ability of chimeric antibody XT-M4 antibody to compete with rat antibody XT-M4 and murine antibody XT-H2 in binding to hRAGE-Fc was determined by antibody competition ELISA binding assay, using biotin-linked XT-M4 and XT-H2 antibodies, in the manner described in Example 7. As shown in FIG. 14, chimeric antibody XT-M4 competes with rat antibody XT-M4 and with murine antibody XT-H2 in binding to hRAGE-Fc.

### Example 14

### Antibody Binding to RAGE of Different Species was Measured by Cell-Based ELISA

Cell Transfection

Human embryonic kidney 293 cells (American Tissue Type Culture, Manassas, Va.) cells were plated at 5×10⁶ cells per 10 cm² tissue culture plate and cultured overnight at 37° C. The next day cells were transfected with RAGE expression plasmids (pAdori1-3 vector encoding mouse, human, baboon, cynomologus monkey or rabbit RAGE) using LF2000 reagent (Invitrogen, Carlsbad Calif.) at a 4:1 ratio of reagent to plasmid DNA using the manufacturers protocol. Cells were harvested 48 hrs post-transfection using trypsin, washed once with phosphate buffered saline (PBS), then suspended in growth media without serum at a concentration of 2×10⁶ cells/ml.

Cell-Based ELISA

Primary antibodies at 1 μg/ml were serially diluted at 1:2 or 1:3 in PBS containing 1% bovine serum albumin (BSA) in a 96-well plate. RAGE-transfected 293 cells or control parental 293 cells (50 μl) at 2×10⁶ cells/ml in serum-free growth medium were added to U-bottom 96 well plate for a final concentration of 1×10⁵ cells/well. The cells were centrifuged at 1600 rpm for 2 minutes. The supernatants were gently discarded by hand with a one-time swing and the plate was patted gently to loose the cell pellet. The diluted primary anti-RAGE antibodies or isotype-matching control antibodies (100 μl) in cold PBS containing 10% fetal calf serum (FCS) were added to the cells and incubated on ice for 1 hour. The cells were stained with 100 μl of diluted secondary anti-IgG antibody HRP conjugates (Pierce Biotechnology, Rockford, Ill.) on ice for 1 hour. Following each step of primary antibody and secondary antibody incubations, the cells were washed 3 times with ice-cold PBS. 100 μl of substrate TMB1 component (BIO FX, TMBW-0100-01) was added to the plate and incubated for 5-30 minutes at room temperature. The color development was stopped by adding 100 μl of 0.18M H₂SO₄. The cells were centrifuged and the supernatants are transferred to a fresh plate and read at 450 nm (Soft MAX pro 4.0, Molecular Devices Corporation, Sunnyvale, Calif.).

The abilities of antibodies chimeric XT-M4 and XT-M4 to bind to human & baboon RAGE as determined by cell-based ELISA are shown in FIG. 14. The EC50 values for the binding of chimeric antibody XT-M4 and XT-M4 to cell surface human, baboon, monkey, mouse & rabbit RAGE expressed by 293 cells, as determined by cell-based ELISA, are shown in Table 7.

TABLE 7
EC50 values for binding to RAGE
determined by cell-based ELISA

| | chimeric XT-M4 | rat XT-M4 |
|---|---|---|
| 293-murine RAGE | ˜1.5 nM | ˜2.2 nM |
| 293-human RAGE | ˜0.8 nM | ˜0.84 nM |
| 293-cyno monkey RAGE | ˜1.66 nM | ˜2.33 nM |
| 293-baboon RAGE | ˜1.25 nM | ˜1.33 nM |

### Example 15

### Binding to RAGE of Different Species—Determined by Immunohistochemical Staining

The abilities of the chimeric antibody XT-M4, the rat XT-M4 antibody, and murine antibodies XT-H1, XT-H2, and XT-H5 to bind to endogenous cell surface RAGE in lung tissue of human, cynomologus monkey, baboon, and rabbit were determined by immunohistochemical (IHC) staining of lung tissue sections.

Stably transfected Chinese Hamster Ovary (CHO) cells were engineered to express murine and human RAGE full length proteins. The murine and human RAGE cDNAs were cloned into the mammalian expression vector, linearized and transfected into CHO cells using lipofectin (methods (Kaufman, R. J., 1990, Methods in Enzymology 185:537-66; Kaufman, R. J., 1990, Methods in Enzymology 185:487-511;Pittman, D. D. et al., 1993, Methods in Enzymology 222: 236). Cells were further selected in 20 nM methotrexate and cell extracts were harvested from individual clones and analyzed by SDS sodium dodecyl sulfate-polyacrylamide gel electrophoresis (SDS-PAGE) and Western blotting to confirm expression.

Immunohistochemistry for RAGE lung tissues isolated from baboon, cynomolgus monkey, rabbit or Chinese Hamster Ovary cells over-expressing human RAGE or control CHO cells were performed using standard techniques. RAGE antibodies and rat IgG2b isotype control or mouse isotype control were used at 1-15 mg. Chimeric XT-M4, XT-M4-hVH-V2.0-2m/hVL-V2.10, XT-M4-hVH-V2.0-2m/hVL-V2.11, XT-M4-hVH-V2.0-2m/hVL-V2.14 were biotinalyted and Sigma IgG1 biotinalyted control antibody at 0.2, 1, 5 and 10 μg/ml was used. Following detection with HRP and Alexa Fluor 594, Alexa Fluor 488 or anti-biotin conjugated with FITC, sections were also stained with 4′-6-Diamidino-2-phenylindole (DAPI).

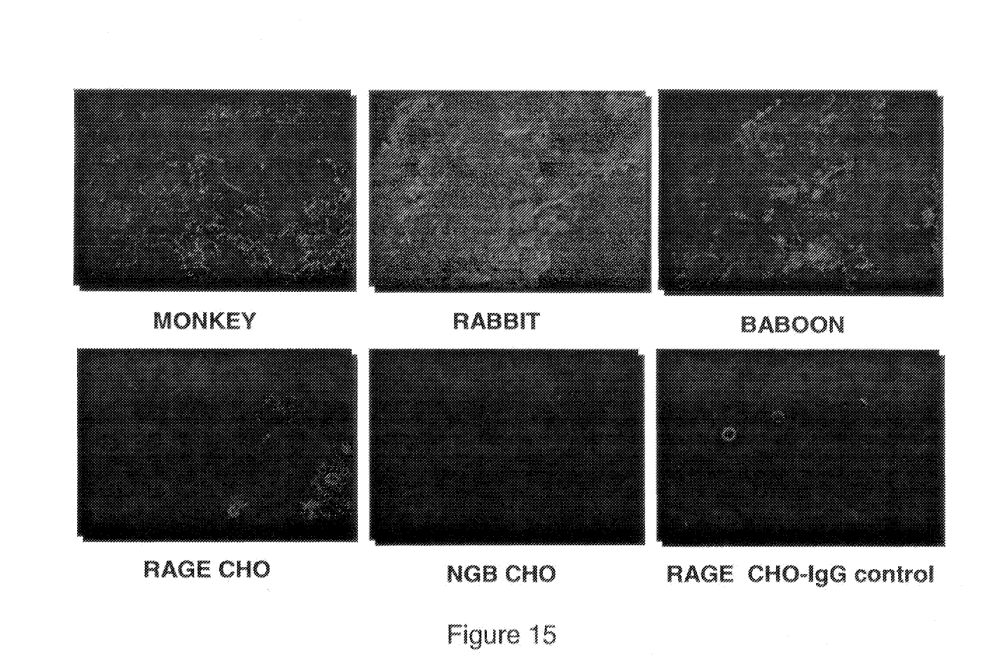

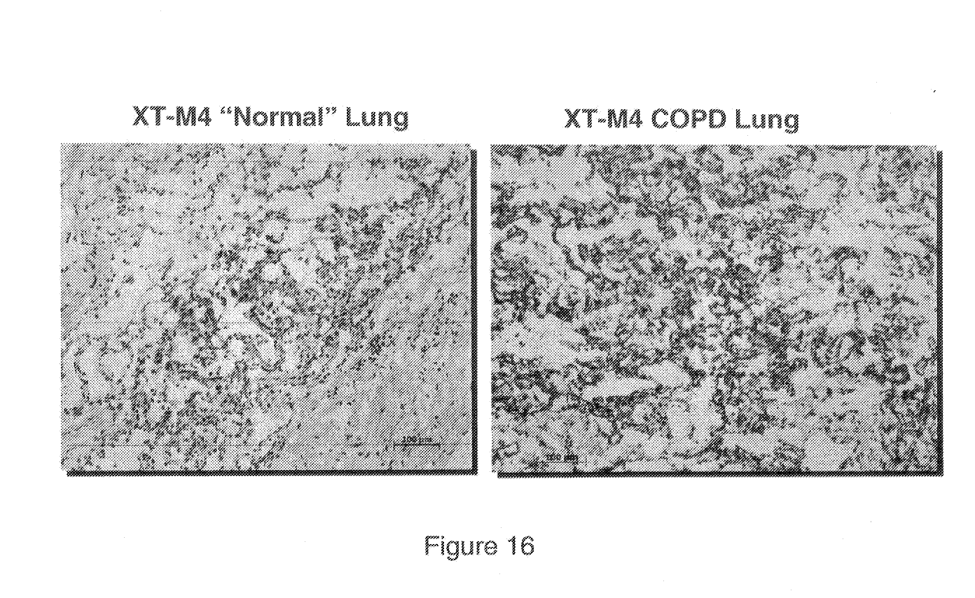

FIG. 15 shows that the chimeric antibody XT-M4 binds to RAGE in lung tissues of cynomologus monkey, rabbit, and baboon. Positive IHC-staining patterns are visible in the samples in which RAGE-producing cells are contacted with chimeric XT-M4, but not in samples in which either RAGE or a RAGE-binding antibody are absent. FIG. 16 shows that the rat antibody XT-M4 binds to RAGE in normal human lung and lung of a human with chronic obstructive pulmonary disease (COPD). The binding of rat XT-M4 antibody and murine antibodies XT-H1, XT-H2, and XT-H5 to endogenous cell surface RAGE in septic baboon lung and normal cynomologus monkey lung, as determined by IHC staining of lung tissue sections, is summarized in Table 8. CHO cells stable transfected with an expression vector that expresses DNA encoding hRAGE is used as a positive control.

TABLE 8
Binding to RAGE in non-human primate lung - assayed by IHC

| | | | | | | | | hRAGE | |
|---|---|---|---|---|---|---|---|---|---|
| | Baboon lung (septic) | | | | Monkey lung (normal) | | | CHO | CHO |
| μg/ml | 1 | 5 | 10 | 15 | 5 | 10 | 15 | 1 | 1 |
| XT-M4 | +++ | +++ | ++++ | ++++ | + | ++ | | +++ | − |
| XT-H1 | ++++ | ++++ | ++++ | ++++ | +++ | +++ | | +++ | − |
| XT-H2 | − | − | + | ++ | − | − | | +++ | − |
| XT-H5 | ++++ | ++++ | ++++ | ++++ | − | − | | +++ | − |
| mRA109 | | | − | − | | − | − | − | − |
| control | | | | | | | | | |
| rSFR | | | − | − | | − | − | − | − |
| control | | | | | | | | | |

### Example 16

### Molecular Modeling for Humanizing Murine Anti-Human RAGE Antibody XT-H2

Molecular modeling of murine anti-human RAGE antibody XT-H2 HV domain Antibody structure templates for modeling murine XT-H2 heavy chain were selected based BLASTP search against Protein Data Bank (PDB) sequence database. Molecular model of murine XT-H2 was built based on 6 template structures, 1SY6 (anti-CD3 antibody), 1MRF (anti-RNA antibody), and 1RIH (anti-tumor antibody) using the Homology module of InsightII (Accelrys, San Diego). The structurally conserved regions (SCRs) of the templates were determined based on the Cα distance matrix for each molecule and the templates structures were superposed based on minimum RMS deviation of corresponding atoms in SCRs. Sequence of the target protein rat XT-H2 VH was aligned to the sequences of the superposed template proteins and the atomic coordinates of the SCRs were assigned to the corresponding residues of the target protein. Based on the degree of sequence similarity between the target and the templates in each of the SCRs, coordinates from different templates were used for different SCRs. Coordinates for loops and variable regions not included in the SCRs were generated by Search Loop or Generate Loop methods as implemented in the Homology module.

Briefly, the Search Loop method scans protein structures that would mimic the region between 2 SCRs by comparing the Cα distance matrix of flanking SCR residues with a pre-calculated matrix derived from protein structures that have the same number of flanking residues and an intervening peptide segment of a given length. The output of the Search Loop method was evaluated to first find a match having minimal RMS deviations and maximum sequence identity in the flanking SCR residues. Then an evaluation of sequence similarity between the potential matches and the sequence of the target loop was undertaken. The Generate Loop method generates atom coordinates de novo was used in those cases where Search Loops did not find optimal matches. Conformation of amino acid side chains was kept the same as that in the template if the amino acid residue was identical in the template and the target. However, a conformational search of rotamers was performed and the energetically most favorable conformation was retained for those residues that are not identical in the template and target. To optimize the splice junctions between two adjacent SCRs, whose coordinates were adapted from different templates, and those between SCRs and loops, the Splice Repair function of the Homology module was used. The Splice Repair sets up a molecular mechanics simulation to derive optimal bond lengths and bond angles at junctions between 2 SCRs or between SCR and a variable region. Finally the model was subjected to energy minimization using Steepest Descents algorithm until a maximum derivative of 5 kcal/(mol Å) or 500 cycles and Conjugate Gradients algorithm until a maximum derivative of 5 kcal/(mol Å) or 2000 cycles. Quality of the model was evaluated using ProStat/Struct_Check utility of the Homology module.

Molecular Modeling of Humanized Anti-RAGE XT-H2 HV Domain

A molecular model of the humanized (CDR grafted) anti-RAGE antibody XT-H2 heavy chain was built with Insight II following the same procedure as described for the modeling of the mouse XT H2 antibody heavy chain, except that the templates used were different. The structure templates used in this case were 1L7I (anti-Erb B2 antibody), 1FGV (anti-CD18 antibody), 1JPS (anti-tissue factor antibody) and 1N8Z (anti-Her2 antibody).

Model Analysis and Prediction of Frame Work Back Mutations-Humanization

The parental mouse antibody model was compared to the model of the CDR-grafted humanized version with respect to similarities and differences in one or more of the following features: CDR-framework contacts, potential hydrogen bonds influencing CDR conformation, and RMS deviations in various regions such as framework 1, framework 2, framework 3, framework 4 and the 3 CDRs.

The following back mutations singly and in combinations were predicted to be important for successful humanization by CDR grafting: E46Y, R72A, N77S, N74K, R67K, K76S, A23K, F68A, R38K, A40R.

### Example 17

### Molecular Modeling for Humanizing Rat Anti-RAGE Antibody XT-M4

Molecular Modeling of Rat Anti-Murine RAGE Antibody XT-M4 HV Domain

Antibody structure templates for modeling rat XT-M4 heavy chain were selected based upon BLASTP search against Protein Data Bank (PDB) sequence database. Molecular models of rat XT-M4 were built based on 6 template structures, 1QKZ (anti-peptide antibody), 1IGT (anti-canine lymphoma monoclonal antibody), 8FAB (anti-p-azophenyl arsonate antibody), 1MQK (anti-cytochrome C oxidase antibody), 1 H0D (anti-angiogenin antibody), and 1MHP (anti-alpha1beta1 antibody) using the Homology module of InsightII (Accelrys, San Diego). The structurally conserved regions (SCRs) of the templates were determined based on the Cα distance matrix for each molecule and the templates structures were superposed based on minimum RMS deviation of corresponding atoms in SCRs. The sequence of the target protein rat XT-M4 VH was aligned to the sequences of the superposed template proteins and the atomic coordinates of the SCRs were assigned to the corresponding residues of the target protein. Based on the degree of sequence similarity between the target and the templates in each of the SCRs, coordinates from different templates were used for different SCRs. Coordinates for loops and variable regions not included in the SCRs were generated by Search Loop or Generate Loop methods as implemented in the Homology module.

Briefly, the Search Loop method scans protein structures that would mimic the region between 2 SCRs by comparing the Cα distance matrix of flanking SCR residues with a pre-calculated matrix derived from protein structures that have the same number of flanking residues and an intervening peptide segment of a given length. The output of the Search Loop method was evaluated to first find a match having minimal RMS deviations and maximum sequence identity in the flanking SCR residues. Then an evaluation of sequence similarity between the potential matches and the sequence of the target loop was undertaken. The Generate Loop method generates atom coordinates de novo was used in those cases where Search Loops did not find optimal matches. Conformation of amino acid side chains was kept the same as that in the template if the amino acid residue was identical in the template and the target. However, a conformational search of rotamers was performed and the energetically most favorable conformation was retained for those residues that are not identical in the template and target. To optimize the splice junctions between two adjacent SCRs, whose coordinates were adapted from different templates, and those between SCRs and loops, the Splice Repair function of the Homology module was used. The Splice Repair sets up a molecular mechanics simulation to derive optimal bond lengths and bond angles at junctions between 2 SCRs or between SCR and a variable region. Finally the model was subjected to energy minimization using Steepest Descents algorithm until a maximum derivative of 5 kcal/(mol Å) or 500 cycles and Conjugate Gradients algorithm until a maximum derivative of 5 kcal/(mol Å) or 2000 cycles. Quality of the model was evaluated using ProStat/Struct_Check utility of the Homology module.

XT-M4 Light Chain Variable Domain

Structural models for XT M4 light chain variable domain were generated with Modeler 8v2 using 1K6Q (anti-tissue factor antibody), 1WTL, 1D5B (antibody AZ-28) and 1BOG (anti-p24 antibody) as the templates. For each target, out of the 100 initial models, one model with the lowest restraint violations, as defined by the molecular probability density function, was chosen for further optimization. For model optimization an energy minimization cascade consisting of Steepest Descent, Conjugate Gradient and Adopted Basis Newton Raphson methods was performed until an RMS gradient of 0.01 was satisfied using Charmm 27 force field (Accelrys Software Inc.) and Generalized Born implicit solvation as implemented in Discovery Studio 1.6 (Accelrys Software Inc.). During energy minimization, movement of backbone atoms was restrained using a harmonic constraint of 10 mass force.

Molecular Modeling of Humanized Anti-RAGE XT-M4 VH Domain

A molecular model of the humanized (CDR grafted) anti-RAGE XT M4 antibody heavy chain was built with Insight II following the same procedure as described for the modeling of the rat XT M4 antibody heavy chain, except that the templates used were different. The structure templates used in this case were 1MHP (anti-alpha1beta1 antibody), 1IGT (anti-canine lymphoma monoclonal antibody), 8FAB (anti-p-azophenyl arsonate antibody), 1MQK (anti-cytochrome C oxidase antibody) and 1H0D (anti-angiogenin antibody).

Humanized XT-M4 Light Chain Variable Domain

A molecular model of the humanized (CDR grafted) anti-RAGE XT M4 antibody light chain was built using Modeler 8v2 following the same procedure as described for the modeling of the rat XT M4 antibody light chain, except that the templates used were different. Structure templates used in this case were 1B6D,1FGV (anti-CD18 antibody), 1UJ3 (anti-tissue factor antibody) and 1WTL as the templates.

Model Analysis and Prediction of Frame Work Back Mutations-Humanization

The parental rat antibody model was compared to the model of the CDR-grafted humanized version with respect to similarities and differences in one or more of the following features: CDR-framework contacts, potential hydrogen bonds influencing CDR conformation, RMS deviations in various regions such as framework 1, framework 2, framework 3, framework 4 and the 3 CDRs, and calculated energies of residue-residue interactions. The potential back mutation(s) identified were incorporated, singly or in combinations, into another round of models built using either Insight II or Modeler 8v2 and the models of the mutants were compared to the parental rat antibody model to evaluate the suitability of mutants in silico.

The following back mutations singly and in combinations were predicted to be important for successful humanization by CDR grafting:

Heavy chain: L114M, T113V and A88S;

Light chain: K45R, L46R, L47M, D70I, G66R, T85D, Y87H, T69S, Y36F, F71Y.

### Example 18

### Humanized Variable Regions with the CDRs of Murine XT-H2 and Rat XT-M4 Antibodies

Humanized heavy chain variable regions were prepared by grafting the CDRs of the murine XT-H2 and rat XT-M4 antibodies onto human germline framework sequences shown in Table 9, and introducing selected back mutations.

TABLE 9

| Antibody | Isotype | Human Germline | Identity |
|---|---|---|---|
| XT-H2_VH | mG1/K | DP-75 VH1; 1-46 | 77.50% |
| XT-M4_VH | rG2b/K | DP-54 VH3; 3-07 | 77.50% |
| XT-H2_VL | mG1/K | DPK-12 VK2; A2 | 80.00% |
| XT-M4_VL | rG2b/K | DPK-9 VK1; 02 | 64.50% |

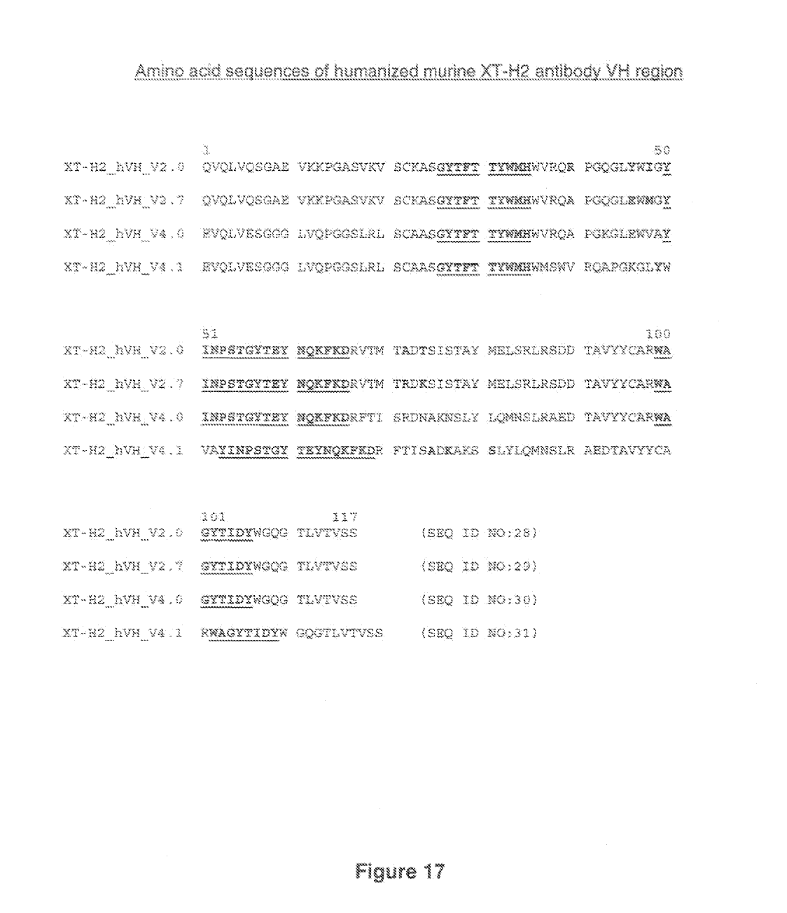

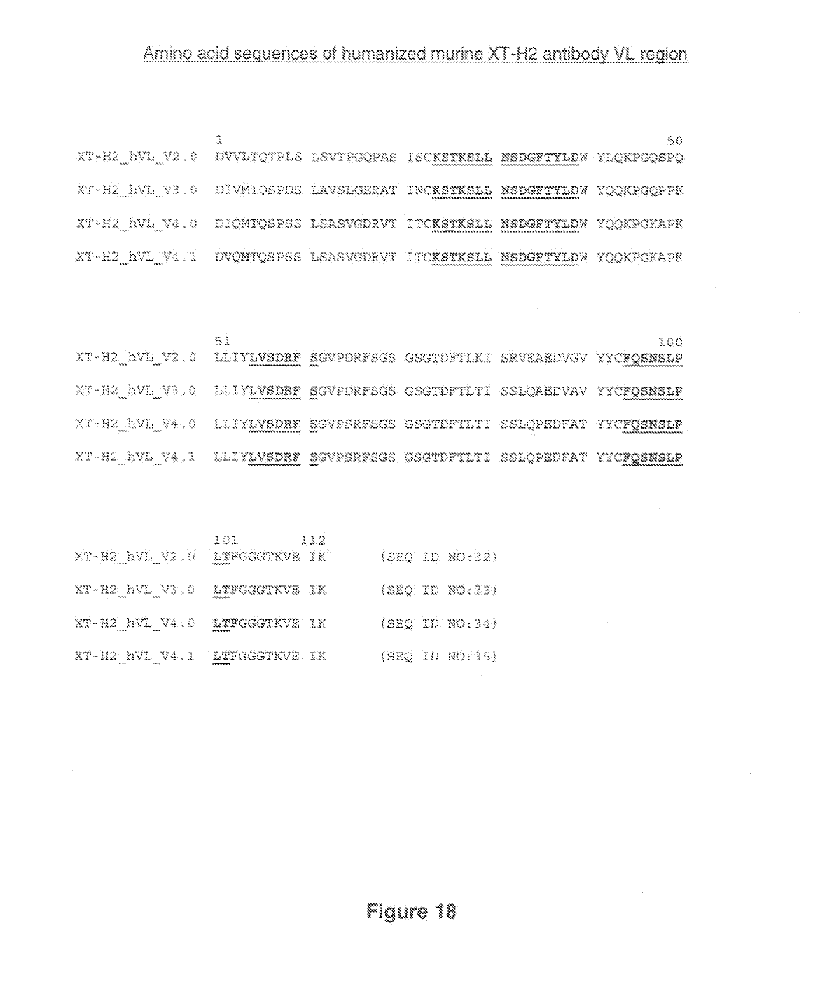

The amino acid sequences of humanized murine XT-H2 heavy and light chain variable regions are shown in FIG. 17 (SEQ ID NOs: 28-31) and FIG. 18 (SEQ ID NOs: 32-35), respectively.

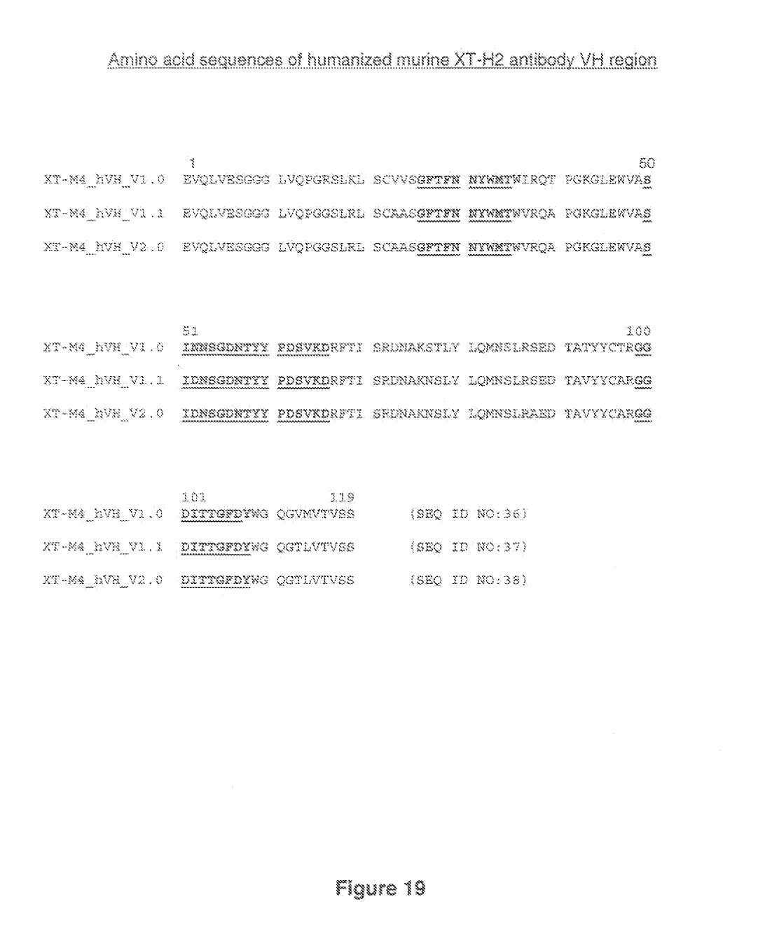

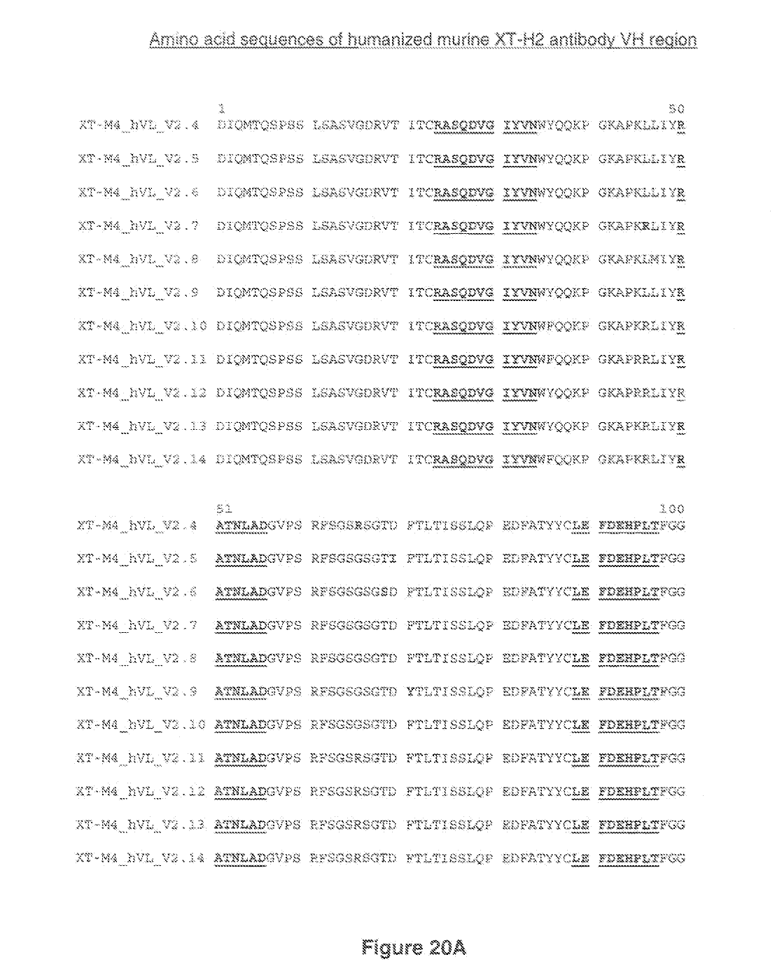

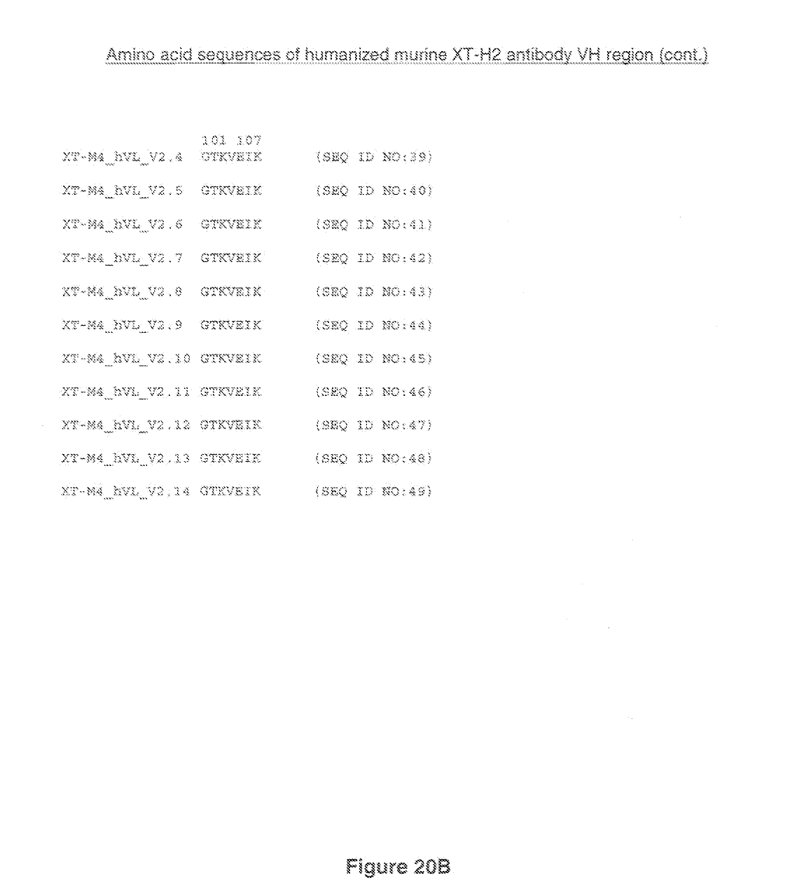

The amino acid sequences of humanized rat XT-M4 heavy and light chain variable regions are shown in FIG. 19 (SEQ ID NOs: 36-38) and FIGS. 20A-20B (SEQ ID NOs: 39-49), respectively.

Germline sequences from which the framework sequences were derived and specific backmutations in the humanized variable regions are identified in Table 10.

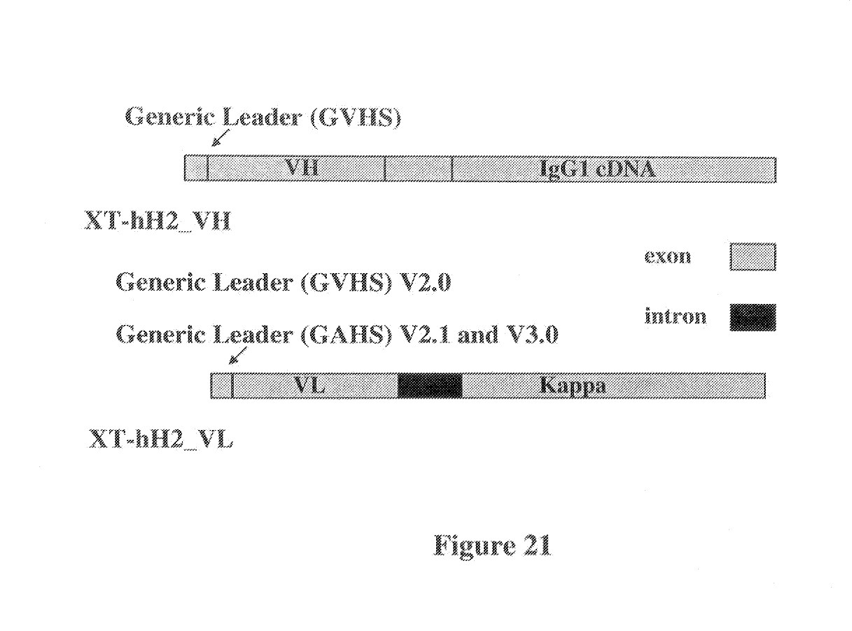

DNA sequences encoding the humanized variable regions were subcloned into expression vectors containing sequences encoding human immunoglobulin constant regions, and DNA sequences encoding the full-length light and heavy chains were expressed in COS cells, using standard procedures. DNAs encoding heavy chain variable regions were subcloned into a pSMED2hlgG1m_(L234, L237)cDNA vector, producing humanized IgG1 antibody heavy chains. DNAs encoding light chain variable regions were subcloned into a pSMEN2 hkappa vector, producing humanized kappa antibody light chains. See FIG. 21.

TABLE 10

| Humanized V domain | Germline | Backmutations |
|---|---|---|
| XT-H2_hVH_V2.0 | DP-75 | A40R, E46Y, M48I, |
| | | R71A, and T73K |
| XT-H2_hVH_V2.7 | DP-75 | |
| XT-H2_hVH_V4.0 | DP-54 FW, VH 3, JH4 | |
| XT-H2_hVH_V4.1 | DP-54 FW, VH 3, JH4 | |
| XT-H2_hVL_V2.0 | DPK-12 | I2V, M4L and P48S |
| XT-H2_hVL_V3.0 | DPK-24 | |
| XT-H2_hVL_V4.0 | DPK-9 Vk1 | |
| XT-H2_hVL_V4.1 | DPK-9 Vk1, Jk 4 | |
| XT-M4_hVH_V1.0 | DP-54, VH3; 3-07 | |
| XT-M4_hVH_V1.1 | DP-54, VH3; 3-07 | |
| XT-M4_hVH_V1.0 | DP-54, VH3; 3-07 | |
| XT-M4_hVL_V2.4 | DPK-9 Vk1; 02 | G66R |
| XT-M4_hVL_V2.5 | DPK-9 Vk1; 02 | D70I |
| XT-M4_hVL_V2.6 | DPK-9 Vk1; 02 | T69S |
| XT-M4_hVL_V2.7 | DPK-9 Vk1; 02 | L46R |
| XT-M4_hVL_V2.8 | DPK-9 Vk1; 02 | |
| XT-M4_hVL_V2.9 | DPK-9 Vk1; 02 | F71Y |
| XT-M4_hVL_V2.10 | DPK-9 Vk1; 02 | |
| XT-M4_hVL_V2.11 | DPK-9 Vk1; 02 | |
| XT-M4_hVL_V2.12 | DPK-9 Vk1; 02 | |
| XT-M4_hVL_V2.13 | DPK-9 Vk1; 02 | |
| XT-M4_hVL_V2.14 | DPK-9 Vk1; 02 | |

### Example 19

### Competition ELISA Protocol

The binding of humanized XT-H2 and XT-M4 antibodies and of chimeric XT-M4 to human RAGE-Fc was characterized by competition enzyme-linked immunosorbent assay (ELISA). To generate a competitor, parental rat XT-M4 antibody was biotinylated. ELISA plates were coated overnight with 1 ug/ml human RAGE-Fc. Varying concentrations of the biotinylated XT-M4 were added in duplicate to wells (0.11-250 ng/ml), incubated, washed and detected with streptavidin-HRP. The calculated ED50 of biotinylated parental rat XT-M4 was 5 ng/ml. The IC50 of chimeric and each humanized XT-M4 antibody was calculated when competed with 12.5 ng/ml biotinylated parental XT-M4 antibody. Briefly, plates were coated overnight with 1 ug/ml human RAGE-Fc. Varying concentrations of chimeric or humanized antibodies mixed with 12.5 ng/ml biotinylated parental rat XT-M4 were added in duplicate to wells (in the range of 9 ng/ml to 20 ug/ml). Biotinylated parental rat XT-M4 antibodies were detected with streptavidin-HRP and IC50 values were calculated. The IC50 values determined for the humanized antibodies by competition ELISA analysis are shown in Table 11.

TABLE 11
IC50 Values for Humanized XT-M4 Antibodies

| | | IC 50 in competition |
|---|---|---|
| Heavy Chain | Light Chain | ELISA with rat XT-M4, ug/m |
| hVH-V1.0 | hVL-V1.0 | 1.5-2.5 |
| hVH-V1.0 | hVL-V2.0 | 7.5->8.6 |
| hVH-V1.0 | hVL-V2.1 | 1.5-2 |
| hVH-V1.0 | hVL-V2.2 | 1.5-2 |
| hVH-V1.0 | hVL-V2.3 | 4.5-8 |
| hVH-V1.0 | hVL-V2.4 | 4.5-8.5 |
| hVH-V1.0 | hVL-V2.5 | 6.5->20 |
| hVH-V1.0 | hVL-V2.6 | >10.9 |
| hVH-V1.0 | hVL-V2.7 | 4-9.5 |
| hVH-V1.0 | hVL-V2.8 | >17 |
| hVH-V1.0 | hVL-V2.9 | >6.8 |
| hVH-V2.0 | hVL-V1.0 | >9.5 |
| hVH-V2.0 | hVL-V2.0 | 10.4 |
| hVH-V2.0 | hVL-V2.1 | 1.1 |
| hVH-V2.0 | hVL-V2.2 | >1.8 |
| hVH-V2.0 | hVL-V2.3 | 3.3 |
| hVH-V2.0 | hVL-V2.4 | 2.9 |
| hVH-V2.0 | hVL-V2.7 | 8.5 |
| hVH-V2.0 | hVL-V2.10 | 0.95 |
| hVH-V2.0 | hVL-V2.11 | 0.15-1.05 |
| hVH-V2.0 | hVL-V2.12 | 2.7 |
| hVH-V2.0 | hVL-V2.13 | 1.5 |
| hVH-V2.0 | hVL-V2.14 | 0.2 |
| hVH-V2.0 | hVL-V2.10 | 0.3-0.4 |
| hVH-V2.0 | hVL-V2.11 | 0.1-0.45 |
| hVH-V2.0 | hVL-V2.14 | 0.2 |

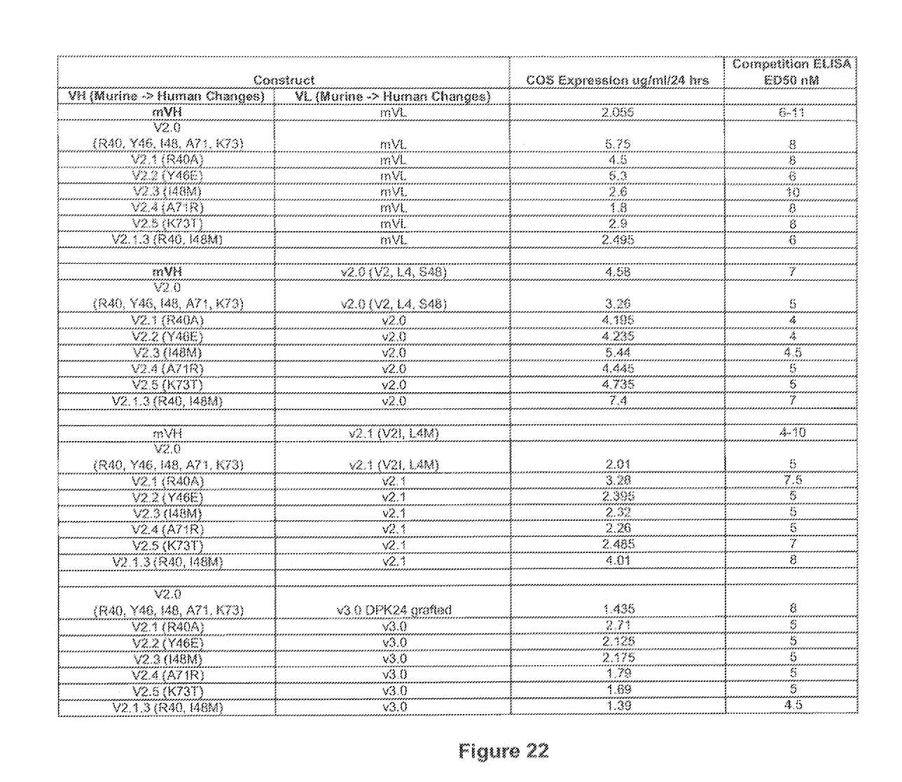

ED50 values for the binding of humanized XT-H2 antibodies to human RAGE-Fc were similarly determined by competition ELISA, and are shown in FIG. 22.

### Example 20

### Cross-Reactivity of Chimeric and Humanized XT-M4 Antibody to Other Cell Surface Receptors

Humanized XT-M4 antibodies XT-M4-hVH-V2.0-2m/hVL-V2.10 and XT-M4-hVH-V2.0-2m/hVL-V2.11, were tested along with chimeric XT-M4 for cross-reactivity with other RAGE-like receptors. These receptors were chosen because they are cell-surface expressed, like RAGE, and their interaction with ligand is similarly dependent on charge. Tested receptors were rhVCAM-1, rhICAM-1-Fc, rhTLR4 (C-terminal His tag), rhNCAM-1, rhB7-H1-Fc mLox1-Fc, hLox1-Fc and hRAGE-Fc (as a positive control). ELISA plates were coated overnight with 1 μg/ml of the listed receptor proteins. Varying concentrations of the above listed humanized and chimeric XT-M4 antibodies were added in duplicate to wells (0.03 to 20 μg/ml), incubated, washed and detected with anti-human IgG HRP. Table 12 shows the results of direct binding ELISA analysis of the binding of chimeric and humanized XT-M4 antibodies to human and mouse cell surface proteins. The data shown are OD450 values for binding detected between receptor and antibody at 20 μg/ml (highest concentration tested).

TABLE 12

| | XT-M4-hVH- | XT-M4-hVH- | |
|---|---|---|---|
| | 2.0-2m/ | V2.0-2m/ | Chimeric |
| | hVL-V2.10 | hVL-V2.11 | XT-M4 |
| rhVCAM-1 | 0.010 | 0.012 | 0.004 |
| rhlCAM-1-Fc | 0.007 | 0.004 | 0.004 |
| rhTLR4 | 0.001 | 0.003 | 0.000 |
| rhNCAM-1 | 0.004 | 0.011 | 0.006 |
| rhB7-H1-Fc | 0.010 | 0.009 | 0.003 |
| mLox1-Fc | 0.016 | 0.010 | 0.010 |
| hLox1-Fc | 0.007 | 0.022 | 0.017 |
| hRAGE-Fc | 3.808 | 3.832 | 3.797 |

### Example 21

### BIACORE™ Binding Assay of Binding to Soluble Human RAGE

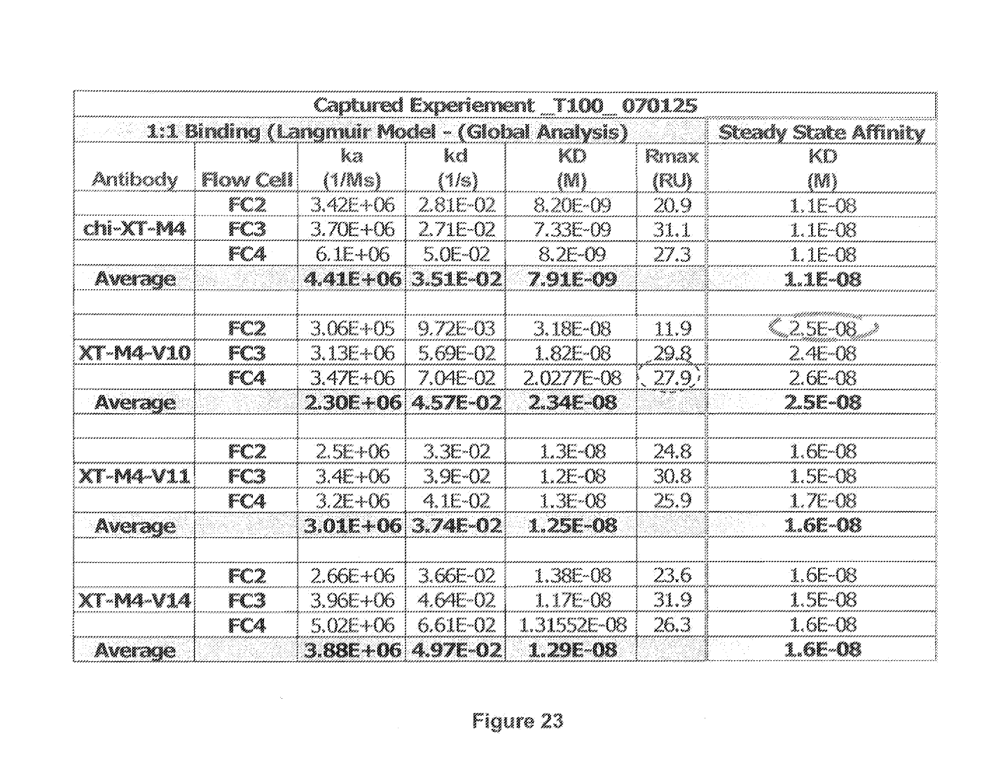

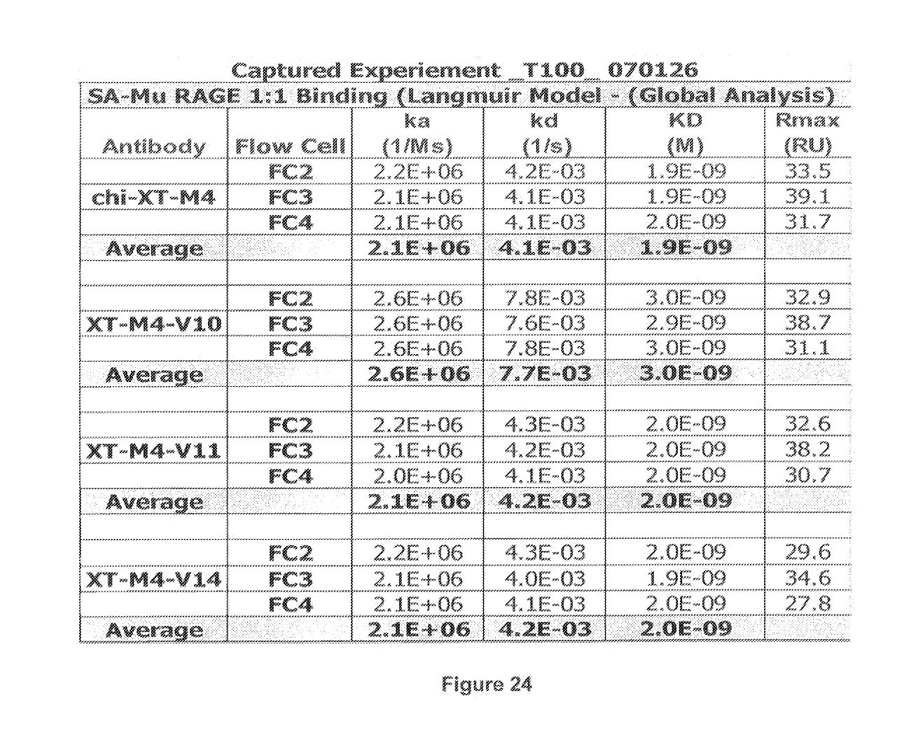

The binding of chimeric antibody XT-M4 and of humanized XT-M4 antibodies to soluble human RAGE (hRAGE-SA) and soluble murine RAGE (mRAGE-SA) was measured by BIACORE™ capture binding assay. The assays were performed by coating anti-human Fc antibodies onto a CM5 BIA chip with 5000 RU (pH 5.0, 7 min.) in flow cells 1-4. Each antibody was captured by flowing at 2.0 μg/ml over the anti-Fc antibodies in flow cells 2-4 (flow cell 1 was used as a reference). Solutions of a purified soluble human streptavidin-tagged RAGE (hRAGE-SA) at concentrations of 100 nM, 50 nM, 25 nM, 12.5 nM, 6.25 nM, 3.125 nM, 1.25 nM and 0 nM were flowed over the immobilized antibodies in duplicate, with dissociation for 5 minutes, and kinetic rate constants (kₐ and k_{d}) and association and dissociation constants (Kₐ and K_{d}) for binding to hRAGE-SA were determined. The results for binding of chimeric XT-M4 and humanized antibodies XT-M4-V10, XT-M4-V11, and XT-M4-V14 for binding to hRAGE-SA and mRAGE-SA are shown in FIGS. 23 and 24, respectively.

### Example 22

### Optimization of Species Cross Reactivity of Lead Antibody XT-H2

Species cross reactivity is engineered by a process of randomly mutating the XT-H2 antibody, generating a library of protein variants and selectively enriching those molecule that have acquired mutations that result in mouse-human RAGE cross reactivity. Ribosome display (Hanes et al., 2000, Methods Enzymol., 328:404-30) and phage display (McAfferty et al., 1989, Nature, 348: 552-4) technologies are used.

Preparing ScFv Antibodies Based on Antibodies XT-H2 and HT-M4

A. ScFv Antibodies Based on XT-H2

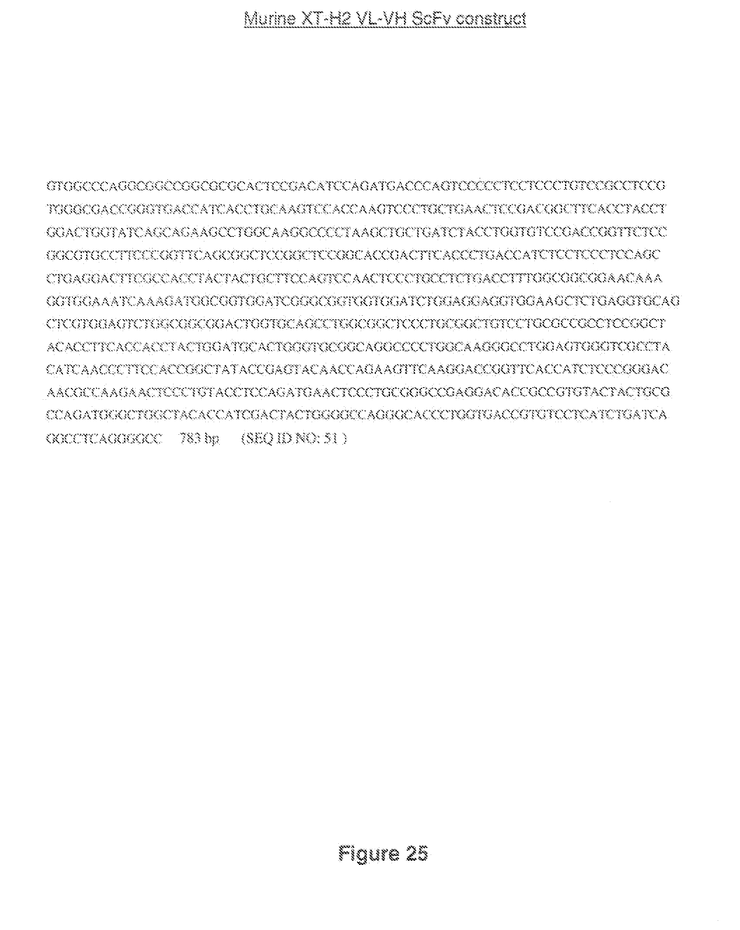

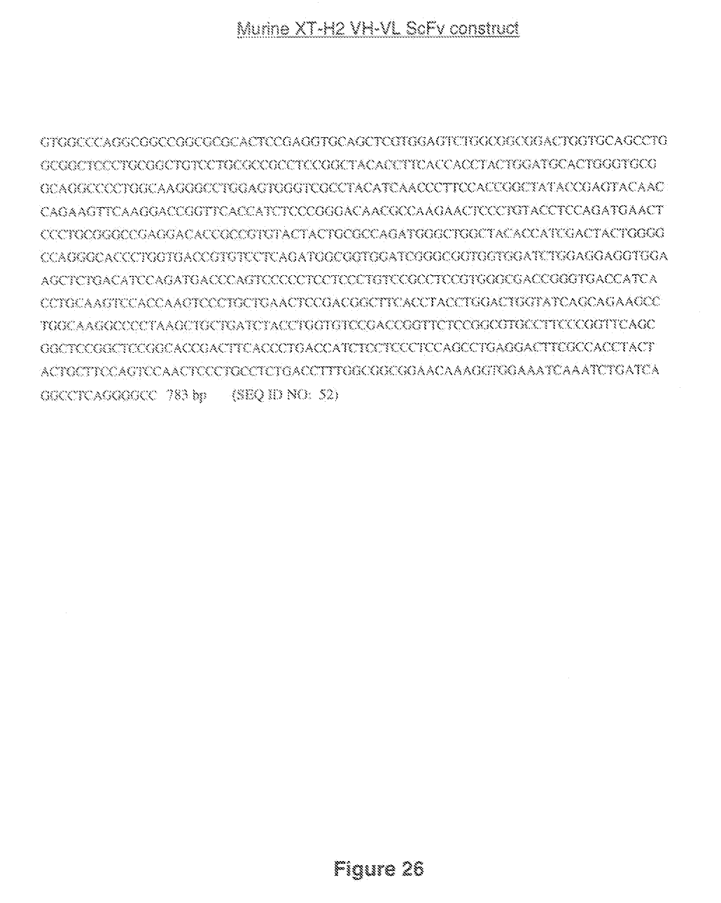

Two ScFv constructs comprising the V regions of XT-H2 were synthesized in either the VH/VL format or the VL/VH format connected by means of a flexible linker of DGGGSGGGGSGGGGSS (SEQ ID NO: 50). The sequences of the ScFv constructs configured as VL-VH and VH-VL are shown in FIG. 25 (SEQ ID NO:51) and FIG. 26 (SEQ ID NO:52), respectively.

B. ScFv Antibodies Based on XT-M4

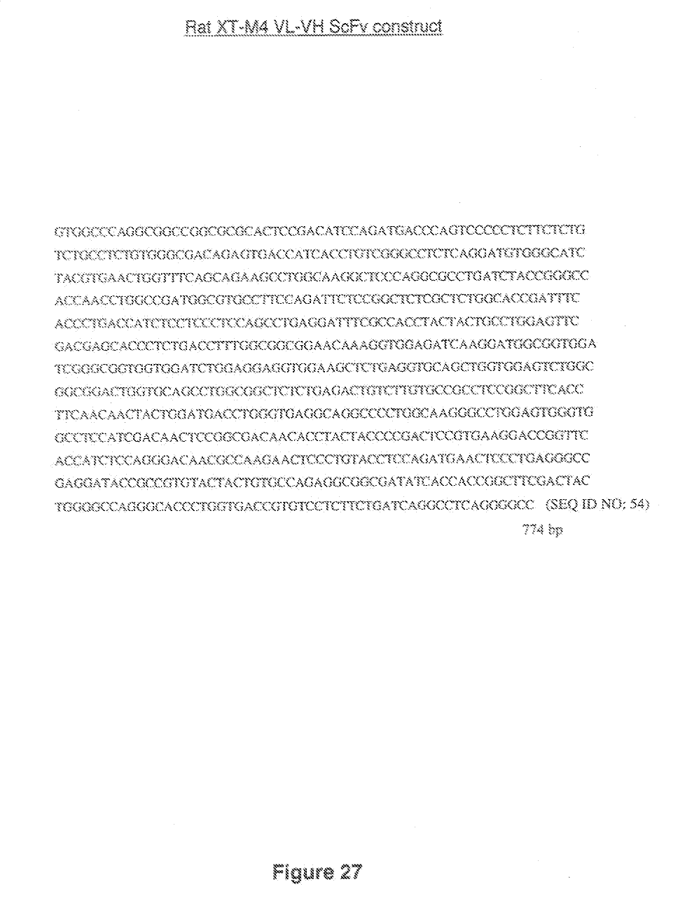

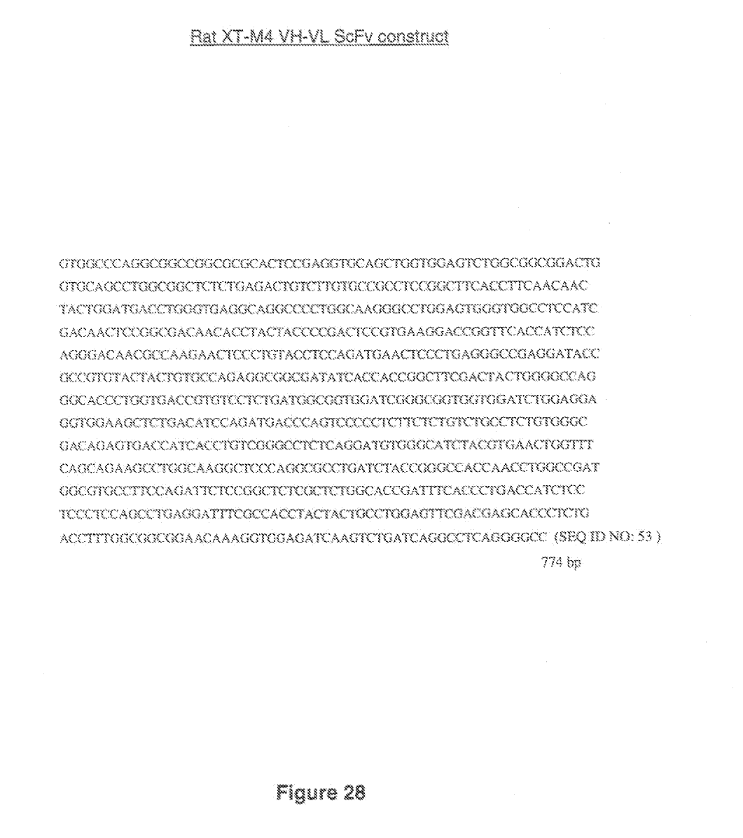

Two ScFv constructs comprising the V regions of XT-M4 were synthesized in either the VH/VL format or the VL/VH format connected by means of a flexible linker of DGGGSGGGGSGGGGSS (SEQ ID NO: 50). The sequences of the ScFv constructs configured as VL-VH and VH-VL are shown in FIG. 27 (SEQ ID NO:54) and FIG. 28 (SEQ ID NO: 53), respectively.

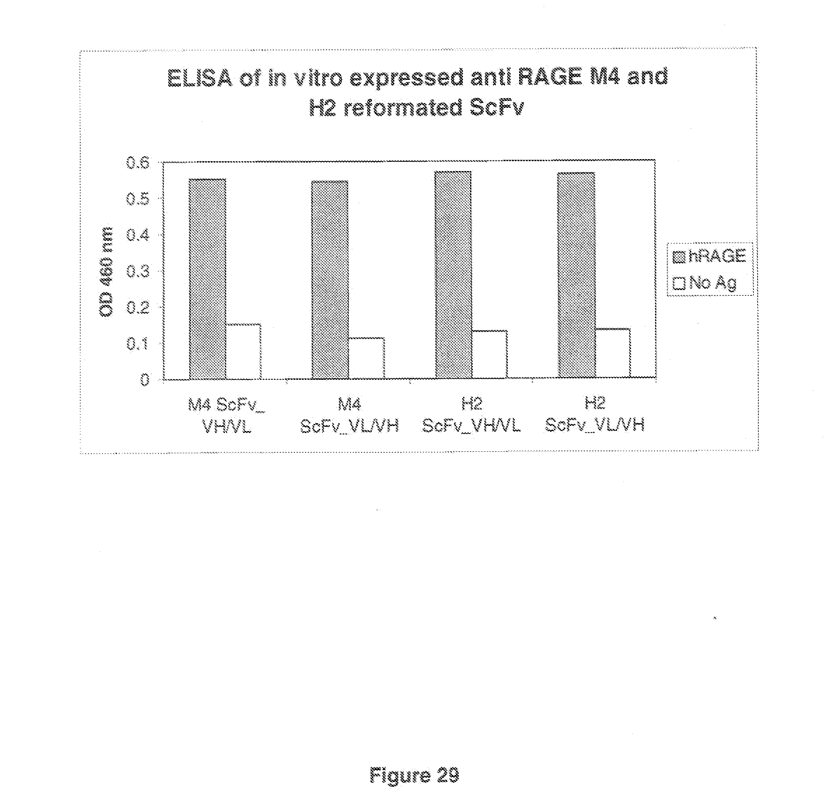

FIG. 29 shows ELISA data of in vitro transcribed and translated M4 and H2 constructs. ELISA plates coated with human RAGE-Fc (5 ug/ml) or BSA (200 ug/ml) in bicarbonate buffer overnight at 4° C., washed with PBS+tween 0.05% and blocked for 1 hour at room temperature with 2% milk powder PBS. Plates were incubated with in vitro translated ScFv for 2 hours at room temp. Plates were blocked and detection was with anti-Flag antibody (1/1000 dilution) followed by rabbit anti-mouse HRP (1/1000 dilution). The data shows that ScFv constructs of the variable regions of the XT-H2 and XT-M4 anti-RAGE antibodies in either the VL/VH or VH/VL configurations can produce functional folded protein that binds specifically to human RAGE. Values for Kd of the ScFv in both formats as determined by BIACORE™ are used to determine the optimum antigen concentrations for selection experiments.

C. Selection and Screening Strategy to Recovery Variants with Improved Mouse/Human RAGE Cross Reactivity

A library of variants is created by error-prone PCR (Gram et al., 1992, PNAS 89:3576-80). This mutagenesis strategy introduces random mutations over the whole length of the ScFv gene. The library is then transcribed and translated in vitro using established procedures (e.g., Hanes et al., 2000, Methods Enzymol., 328:404-30). This library is subjected to round 1 of selection on human-RAGE-Fc, the non-bound ribosomal complexes are washed away, and the antigen-bound ribosomal complexes are eluted. The RNA is recovered, converted to cDNA by RT-PCR and subjected to round 2 of selection on mouse RAGE-Fc. This alternating selection strategy preferentially enriches clones which bind to both human and mouse RAGE-Fc. The output from this selection is then put through a second 2 of error-prone PCR. The library generated is then subjected to round 3 and round selections on human-RAGE-Fc and mouse RAGE-Fc, respectively. This process is repeated as required. The output pools of RNA from each selection step are converted to cDNA and cloned into a protein expression vector pWRIL-1 to evaluate species cross reactivity of variant ScFvs. The pools of diversity are also sequenced to evaluate diversity to determine if selections are moving towards dominant clones that have species cross reactivity.

### Example 23

### Affinity Maturation of Lead Antibody XT-M4

Improved affinity translates into a potential benefit of reduced dose or frequency of dose and/or increased potency. The affinity for hRAGE is improved by affinity maturation, using a combined process of targeted mutagenesis to the VH-CDR3 coupled to random error-prone PCR mutagenesis (Gram et al., 1992, PNAS 89:3576-80). This generates a library of antibody variants from which molecules are recovered that have an improved affinity for human-RAGE whilst maintaining species cross reactivity for mouse-RAGE-Fc. Ribosome display technology (Hanes et al, 1997, supra) and phage display technology (McAfferty et al., 1989, supra) are used.

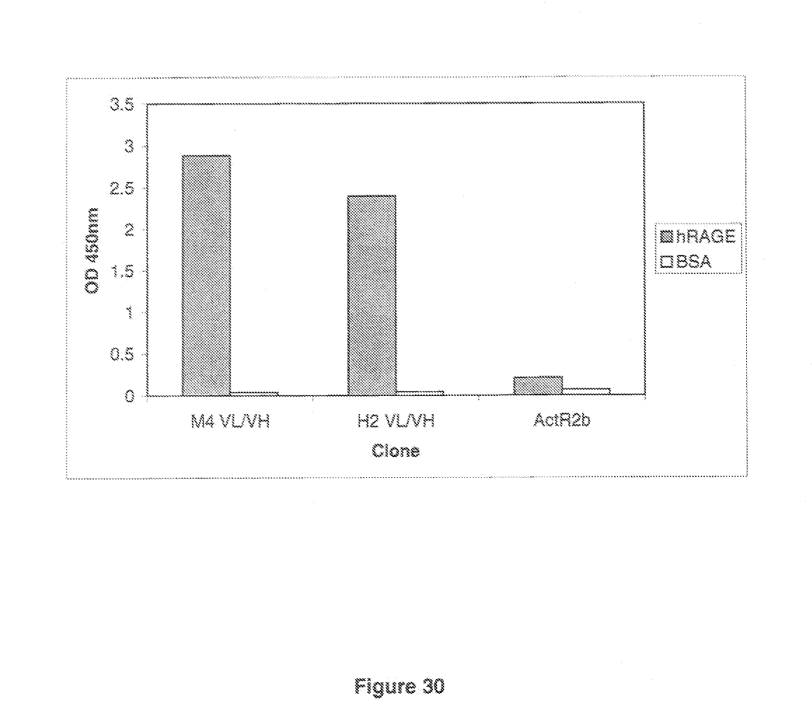

FIG. 30 shows ELISA binding data of XT-M4 and XT-H2 ScFv constructs in pWRIL-1 in the VL-VH format, expressed as soluble protein in Escherichia coli and tested for binding on human RAGE-Fc and BSA. ActRIIb represents a non-binding protein expressed from the same vector as a negative control. ELISA plates were coated with human RAGE-Fc (5 ug/ml) or BSA (200 ug/ml) in bicarbonate buffer overnight at 4oC, washed with PBS+tween 0.05% and blocked for 1 hour at room temperature with 2% milk powder PBS. Periplasmic preparations of 20 ml E. coli cultures were performed using standard procedures. The final volume of periplasmic preparations of unpurified ScFv antibodies was 1 ml of which 50 ul was pre-incubated with anti-His antibody at 1/1000 dilution for 1 hour at room temperature in a total volume of 100 ul with 2% milk powder PBS. The cross linked periplasmic preparations were added to the ELISA plate and incubated for a further 2 hours at room temperature. The plates were washed 2 times with PBS+0.05% tween and 2 times with PBS and incubated with rabbit anti-mouse HRP at 1/1000 dilution in 2% milk powder PBS. The plates were washed as before and binding was detected using standard TMB reagents. The data shows that ScFv constructs of XT-M4 and XT-H2 antibodies in the VL/VH configuration can produce functional folded soluble protein in E. coli that binds specifically to human RAGE. Starting Kd values of the ScFv in both formats as determined by BIACORE™ are used to determine the optimum antigen concentrations for affinity selections.

### Example 24

### Selection and Screening Strategy to Recovery Variants with Improved Affinity for hRAGE-Fc While Maintaining Species Cross Reactivity

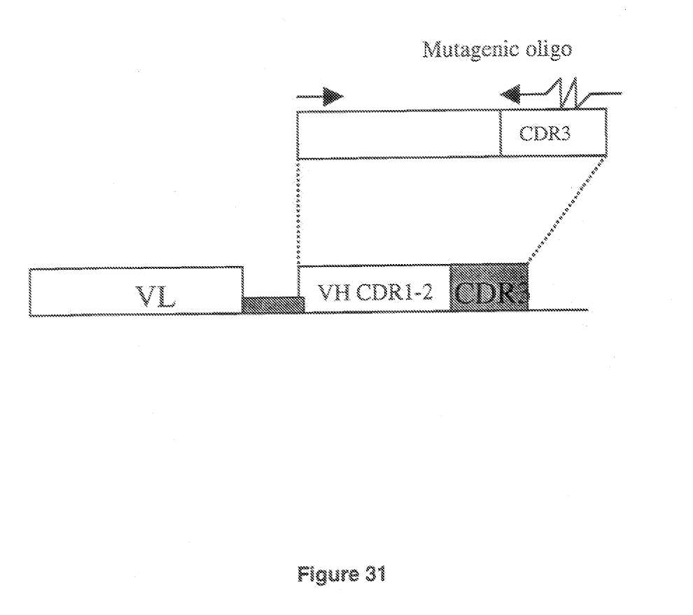

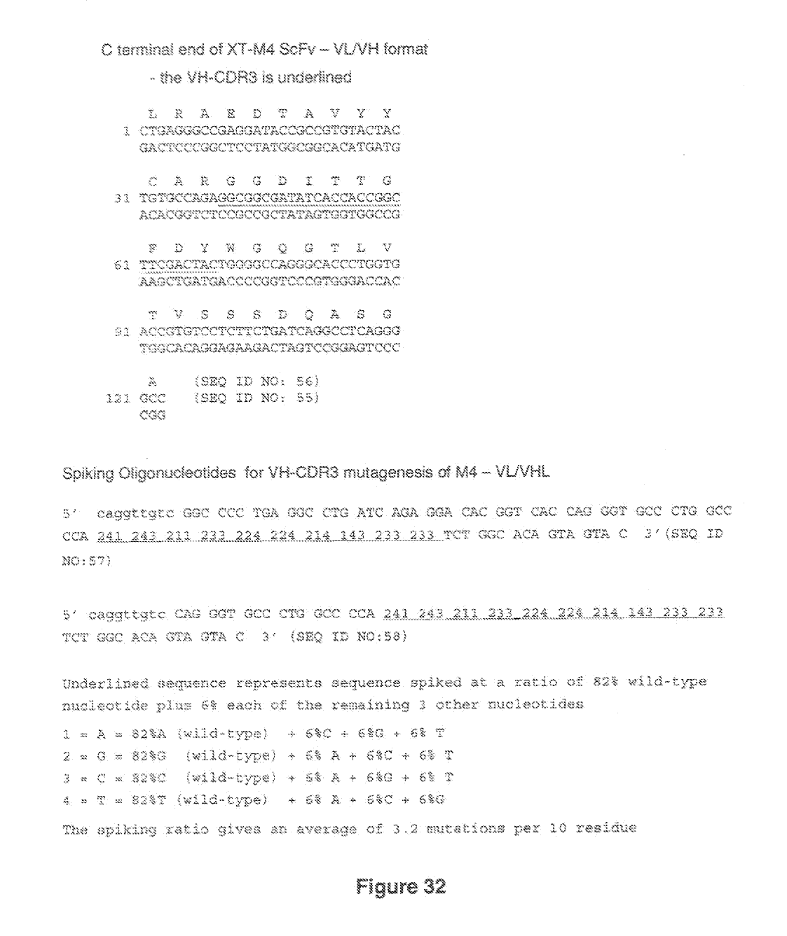

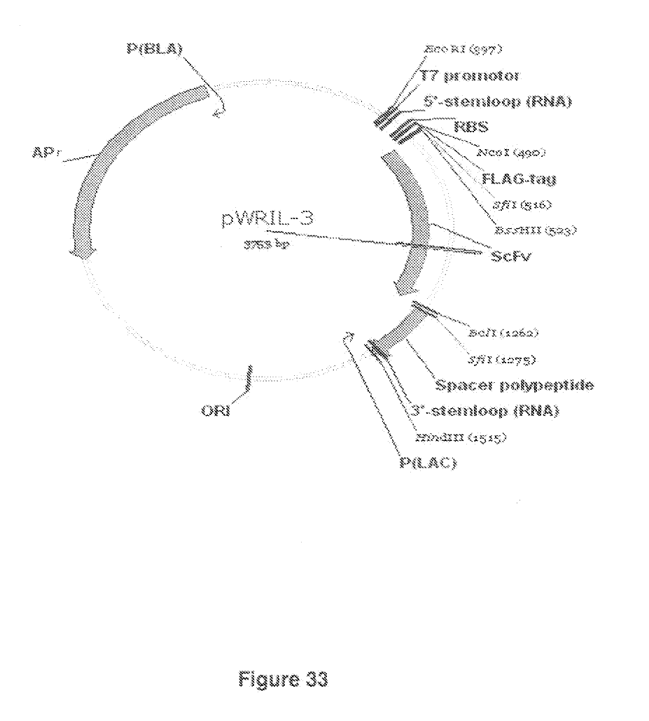

A library of variants is created by spiked mutagenesis of the VH-CDR3 of XT-M4 using PCR. FIG. 31 schematically represents how PCR is used to introduce spiked mutations into a CDR of XT-M4. (1) A spiked oligonucleotide is designed carrying a region of diversity over the length of the CDR loop and bracketed by regions of homology with the target V gene in the FR3 and FR4. (2) The oligonucleotide is used in a PCR reaction with a specific primer that anneals to the 5′ end of target V gene and is homologous to the FR1 region. FIG. 32 shows the nucleotide sequence of the C terminal end of the XT-M4 VL-VH ScFv construct (SEQ ID NO: 56). VH-CDR3 is underlined. Also shown are two spiking oligonucleotides (SEQ ID NOs:57-58) with a number at each mutation site that identifies the spiking ratio used for mutation at that site. The nucleotide compositions of the spiking ratios corresponding to the numbers are also identified.

The XT-M4-VHCDR3 spiked PCR product is cloned into the ribosome display vector pWRIL-3 as a Sfi1 fragment to generate a library. This library is subjected to selection on human biotinylated RAGE using ribosome display (Hanes and Pluckthun., 2000). Biotin labelled antigen is used as this allows for solution based selection which allows for more kinetic control over the process and increases the likelihood of preferentially enriching the higher affinity clones. Selections are performed either in an equilibrium mode at a decreasing antigen concentration relative to starting affinity or in a kinetic mode where improved off rate is specifically selected for using competition with unlabelled antigen over a empirically determined time frame. The non-bound ribosomal complexes are washed away, the antigen bound ribosomal complexes are eluted, the RNA is recovered, converted to cDNA by RT-PCR and a second round of selection on biotinylated mouse-RAGE-Fc is performed to maintain species cross reactivity. The output from this selection step containing ScFv variants with mutations in the VH-CDR3 is then subjected to a cycle 2 step of mutagenesis. This mutagenesis step involves the generation of random mutations using error prone PCR. The library generated is then subjected to round 3 selections on biotinylated human-RAGE-Fc at a 10 fold lower antigen concentration. This process is repeated as required. The output pools of RNA from each selection step are converted to cDNA and cloned into a protein expression vector pWRIL-1 to rank affinity and species cross reactivity of variant ScFv's. The pools of diversity are also sequenced to evaluate diversity to determine if selections are moving towards dominant clones.

### Example 25

### Affinity Maturation of XT-M4 Using Phage Display

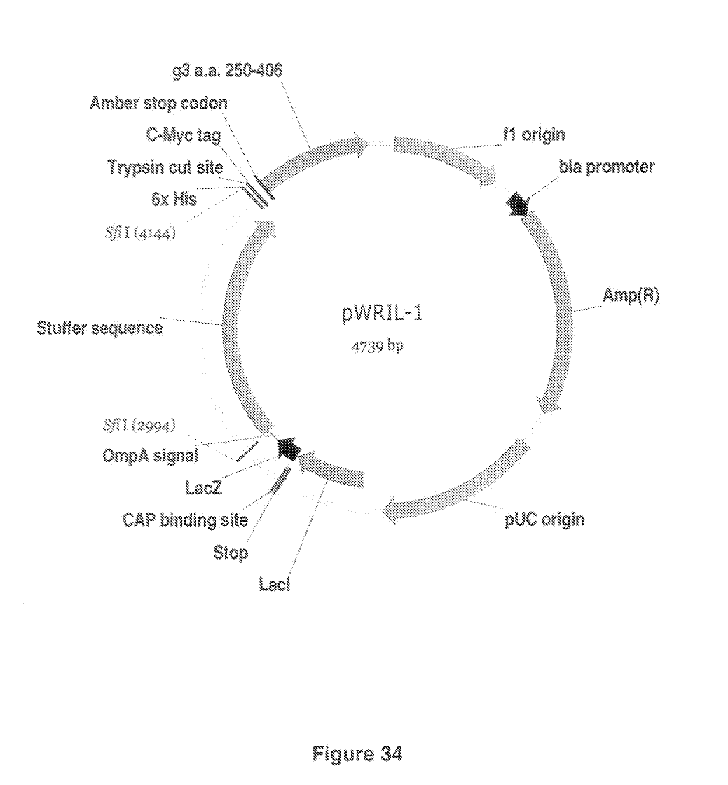

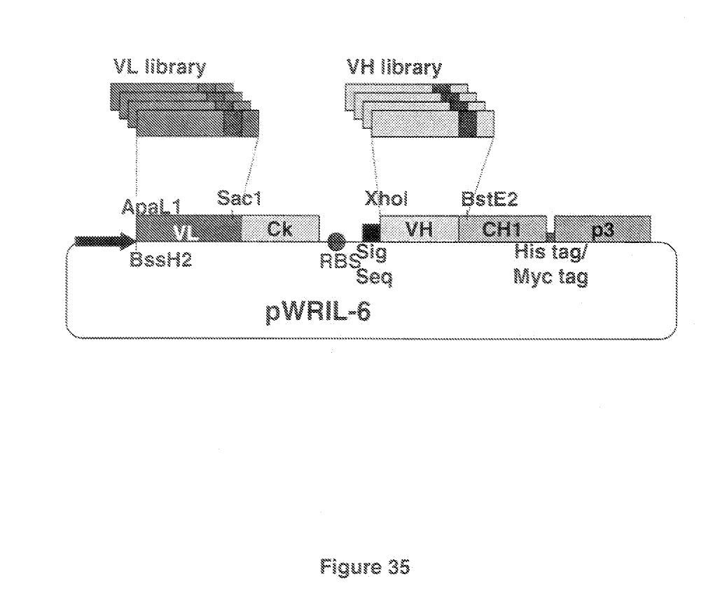

The VH-CDR3 spiked library is cloned into the phage display vector pWRIL-1 shown in FIG. 34 for selection on biotinylated hRAGE. Biotin labelled antigen will be used as this format is more compatible with affinity driven selections in solution. Selections are performed either in an equilibrium mode at a decreasing antigen concentration relative to starting affinity or in a kinetic mode where improved off rate is specifically selected for using competition with unlabelled antigen over an empirically determined time frame. Standard procedures for phage display are used.

ScFv can dimerize, which complicates selection and screening procedures. Dimerized ScFv potentially shows avidity-based binding and this increased binding activity can dominate selections. Such improvements in the ability of ScFv to dimerize rather than in any intrinsic improvement in affinity have little relevance in the final therapeutic antibody, which is generally an IgG. To avoid artifacts resulting from changes in ability to dimerize, Fab antibody formats are used, as they generally do not dimerize. XT-M4 has been reformatted as a Fab antibody and cloned into a new phage display vector pWRIL-6. This vector has restriction sites that span both the VH and VL regions and do not cut frequently in human germline V genes. These restriction sites can be used for shuffling and combinatorial assembly of VL and VH repertoires. In one strategy, VH-CDR3 and VL-CDR3 spiked libraries are both combinatorially assembled in the Fab display vector as shown in FIG. 34, and are selected for improved affinity.

### Example 26

### Physical Characterization of Chimeric Antibody XT-M4

Preliminary characterization by high-performance liquid chromatography (HPLC)/mass spectrometry (MS) peptide mapping and subunit analysis with on-line MS detection have confirmed that the amino acid sequence is as predicted from the chimeric XT-M4 DNA sequence. These MS data also indicated that the expected N-linked oligosaccharide sequence consensus site at Asn²⁹⁹SerThr is occupied and the two major species are complex N-linked biantennary core fucosylated glycans that contain zero or one terminal galactose residues, respectively. In addition to the expected N-linked oligosaccharide located in the Fc region of the molecule, an N-linked oligosaccharide was observed at a sequence consensus site (Asn⁵²AsnSer) in the CDR2 region of the heavy chain of chimeric XT-M4. The extra N-linked oligosaccharide is found primarily on only one of the heavy chains and comprises approximately 38% of the molecules as determined by CEX-HPLC analysis (there may be other acidic species that cannot be differentiated by primary structure, which may contribute to the total percent acidic species). The predominant species is a core fucosylated biantennary structure with two sialic acids. The absorptivity is used to calculate the concentration by measuring A₂₈₀. The theoretical absorptivity of chimeric XT-M4 was calculated to be 1.35 mL mg⁻¹ cm⁻¹.

The apparent molecular weight of chimeric XT-M4 as determined by non-reducing SDS-PAGE is approximately 200 kDa. The antibody migrates more slowly than expected from its sequence. This phenomenon has been observed for all recombinant antibodies analyzed to date. Under reducing conditions, chimeric XT-M4 has a single heavy chain band migrating at approximately 50 kDa and a single light chain migrating at approximately 25 kDa. There is also has an additional band that migrates just above the heavy chain band. This band was characterized by automated Edman degradation and was determined to have an NH₂-terminal that corresponds to the heavy chain of chimeric XT-M4. These results, along with the increase in molecular weight observed by SDS-PAGE, indicate that the additional band is consistent with a heavy chain that has the extra N-linked oligosaccharide in the CDR2 region.

The predicted isoelectric point (pI) of chimeric XT-M4 based on the amino acid sequence is 7.2 (without COOH-terminal Lys in the heavy chain). IEF resolved chimeric XT-M4 into approximately ten bands migrating within a pi range of approximately 7.4-8.3 with one dominant band that migrates with a pi of approximately 7.8. The pI determined by capillary electrophoresis isoelectric focusing was approximately 7.7.

Analysis of development material by cation exchange high performance liquid chromatography (CEX-HPLC) provides further resolution for chimeric XT-M4 species that differ in molecular charge. The majority of the observed charge heterogeneity is most likely due to the contributions from the sialic acids that are found on the additional N-linked oligosaccharide located in CDR2 region of the heavy chain. A minor portion of the charge heterogeneity observed may be attributed to the heterogeneity of COOH-terminal lysine.

### Example 27

### Removal of the Glycosylation Site

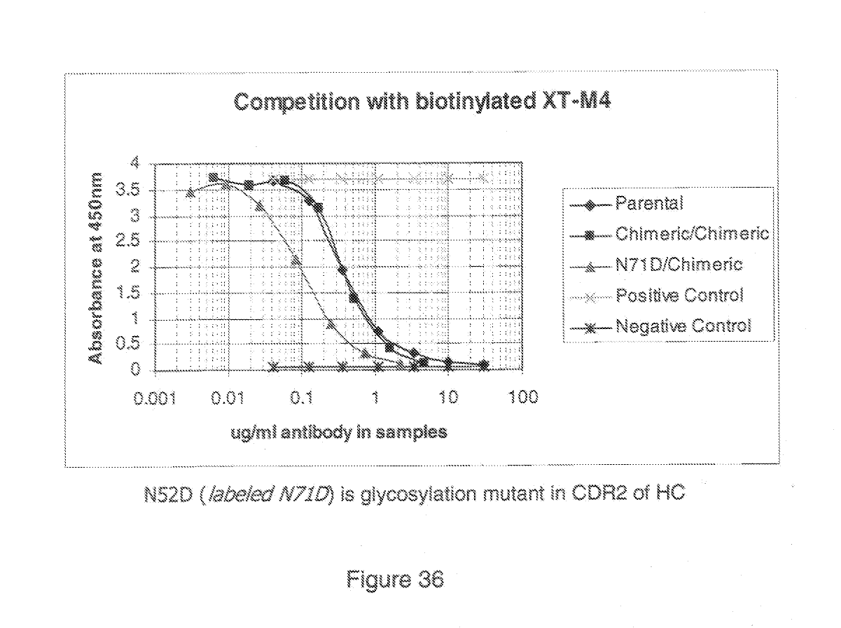

Mutation that converts asparagine (N) to aspartic acid (D) at position 52 (by Kabat numbering) in the heavy chain variable region of antibody XT-M4 improves the binding of the XT-M4 antibody to human RAGE as determined by ELISA analysis of direct binding to hRAGE-Fc, as shown in FIG. 36. The N52D mutation is in CDR2 of the heavy chain variable region of antibody XT-M4.

### Example 28

### In vivo Pre-Clinical Assay of Efficacy of Chimeric Anti-RAGE Antibody

A. Pharmacokinetics (PK)

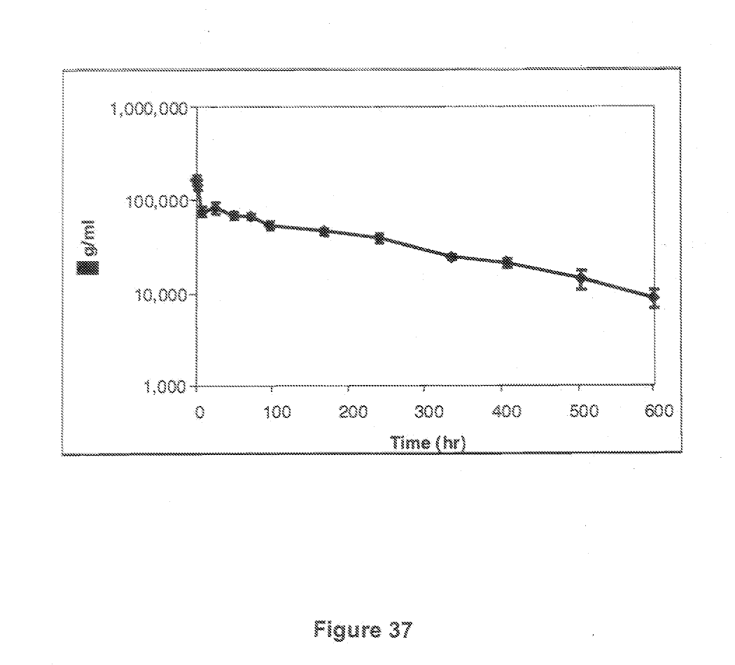

Serum concentration of chimeric antibody chimeric XT-M4 following a single IV dose of 5 mg/kg to male BALB/c mice (n=3) were evaluated for chimeric XT-M4 Serum concentration of antibody over time was measured with an IgG ELISA. The average serum exposure of the chimeric XT-M4 was (23,235 g·hr/mL) and the half-life is approximately one week (152 hours). See FIG. 37.

### Example 29

### CFC Model of Memory Deficit

The contextual fear conditioning (CFC) paradigm was utilized to examine the effect of administering a chimeric rat antibody, XT-M4, that binds specifically to RAGE and inhibits the binding of a RAGE binding partner, on cognitive performance in an animal model of reduced cognitive function due to amyloid deposition.

The Tg2576 model mice develop amyloid plaque by 18 months and this is preceded by LTP deficits in hippocampal CA1 and dentate gyrus, spatial memory deficits in a modified water maze, impaired synaptic plasticity, and an elevation of Aβ aggregates and oligomers by 6 months of age. Aβ induces contextual memory deficits in young, non-plaque bearing Tg2576 mice. Contextual fear conditioning (CFC), a test of hippocampal-dependent learning and memory, was performed in the Tg2576 transgenic human APP mouse model of Aβ formation and amyloid deposition.

Contextual learning involves the association of an aversive stimulus (footshock) with a specific cage environment in which the shock occurred (context). Memory for the conditioning is expressed as context-dependent freezing in the absence of the shock. Mice are conditioned in operant chambers by pairing the context with a brief footshock. Training consists of a 5-minute session in the operant chamber during which the animal receives two mild footshocks. The memory test occurs approximately 24 hours later when the animal is reintroduced to the environment in which it had previously been shocked. Activity levels are recorded during the memory test and the time spent in a “frozen” state, expressed as a percent of the total amount of time, is analyzed by ANOVA across the treatment groups. Decreased levels of activity indicate an intact memory for the aversive event. In contrast to non-transgenic littermates, it was determined that Tg2576 develop contextual memory deficits between the age of 14-16 weeks and that full deficits are observed by 20 weeks of age.

Administration of Antibodies

Chimeric XT-M4 antibodies specific for murine RAGE were diluted in PBS and administered ip to 20 week old Tg2576 and age-matched non-transgenic (wild-type) littermates in single doses (10 mg/kg) on days 1, 4, 7, and 10. A neutral (inactive) antibody was administered as a control. Training of the mice began on day 11, 24 hours after the fourth antibody dose was administered, and testing occurred on day 12.

Memory scores (increased freezing) were examined during the testing session on day 12. Efficacy was determined by demonstrating the reversal of memory deficits relative to PBS-treated transgenic animals. The minimal efficacious dose (MED) was determined by generating dose-response curves with doses ranging from 0.1 to 30 mg/kg. Duration of efficacy following a single immunization at the established MED was determined by time course analysis and evaluating the extension of time intervals prior to training on improved cognition.

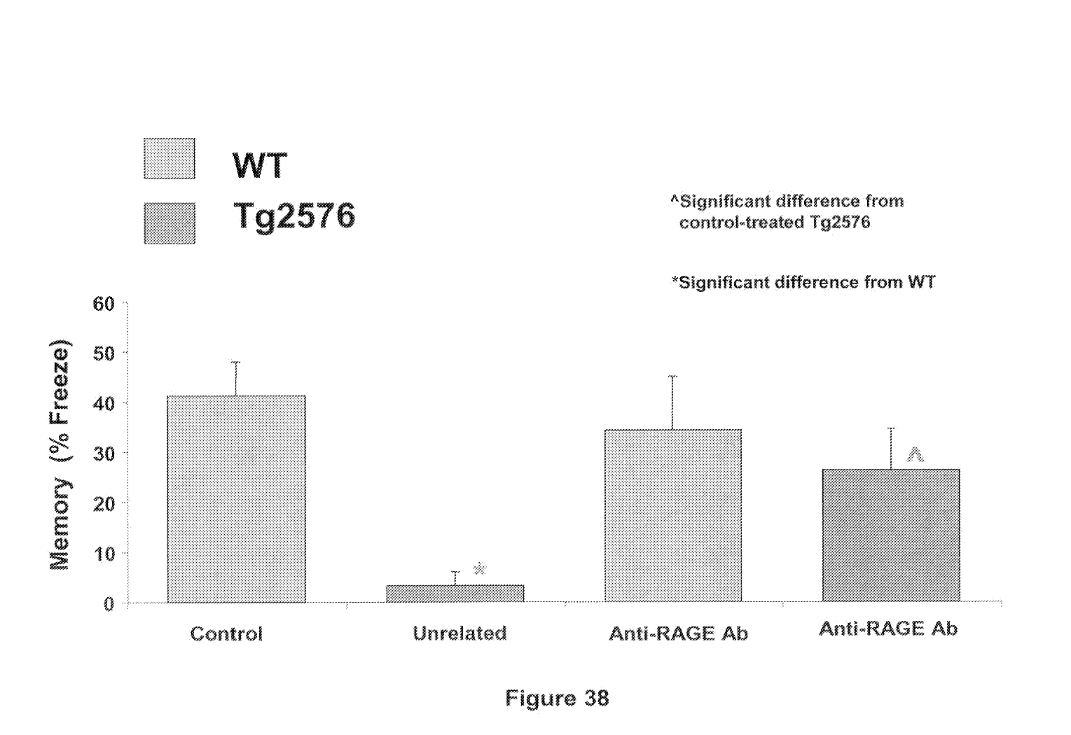

Chimeric XT-M4 antibodies (κ, IgG1) demonstrated a significant reversal of contextual memory deficits. In contrast, mice that were administered an unrelated antibody and PBS controls displayed no effect on CFC in Tg2576 mice or wild type littermates. The data show that administration with chimeric monoclonal XT-M4 antibody against murine RGE is efficacious for improving cognition in the APP transgenic model of Alzheimer's disease. (See FIG. 38)

Materials and Methods

Mice

The mice used in the study were heterozygous male transgenic Tg257618 mice expressing the human APP protein. Genotype was confirmed by PCR and all animals homozygous for the Retinal Degeneration (Rd) mutation were excluded. The background strain consisted of a C57BI6 and 129SJL cross. Contextual fear conditioning studies were performed on mice at 20 weeks of age (n=8-12/genotype/age).

Testing Apparatus

Contextual fear conditioning (CFC) was performed in six 30×24×21 cm operant chambers (Med Associates, Inc., St. Albans, Vt.) constructed from aluminum sidewalls and plexiglass ceiling, door and rear wall. Each chamber was equipped with a floor consisting of 36 stainless steel rods through which a foot shock could be administered. In addition, each chamber had 2 stimulus lights, one house light and a solenoid. Lighting, the footshock (US) and the solenoid (CS) were all controlled by a PC running MED-PC software. The chambers were located in a sound isolated room in the presence of red light.

Contextual Fear Conditioning

Training of the Tg2576 mice or their age-matched wild type littermates began on day 11, one day after the mice received their fourth dose of the chimeric XT-M4 anti-RAGE antibody.

Training consisted of placing the mice in the operant chambers, illuminating both the stimulus and houselights and allowing them to explore for 2 minutes. At the end of the two minutes, the auditory cue (2 Hz clicking via the solenoid; CS) was presented for 15 seconds. The footshock (US; 1.5 mAmp) was administered for the final 2 seconds of the CS and co-terminated with the CS presentation. This procedure was repeated and 30 seconds after the second footshock the mice were removed from the chambers and returned to their homecages.

Twenty hours after training animals were returned to the chambers in which they had previously been trained. Freezing behavior, in the same environment in which they had received the shock (Context), was then recorded by the experimenter using time sampling in 10 seconds bins for 5 minutes (30 sample points). Freezing was defined as the lack of movement except that required for respiration. At the end of the 5 minute, mice were returned to their homecages.

Data on freezing in the Novel and Cue condition was collected after all of the mice had been tested in Context (approximately 60 minutes after the initial Context test). The novel environment consisted of modifications of the operant chamber including an opaque plexiglass divider from the rear right corner to the front left, a plexiglass floor as well as decreased illumination (house light only). Mice were placed in the Novel context and time sampling was used to collect freezing scores for 3 minutes (18 sample points). At the end of the 3 minutes, the auditory clicker (CS) was presented for 3 minutes during which freezing was again scored (18 sample points). Freezing scores for each animal were converted to percent freezing for each portion of the test. Memory for the context (Contextual memory) for each animal was obtained by subtracting the percent freezing in the novel condition (a measure of basal activity) from that observed in the context.

Antibody Treatment

The chimeric XT-M4 antibodies were diluted in PBS (10 mg/kg) and administered ip in 20 week old Tg2576 mice or their age-matched wild type littermates, as single doses on days 1, 4, 7, and 10 with training on day 11 and testing on day 12.

Data Analysis

Contextual memory was analyzed using two-way ANOVA and post hoc Fischer's pairwise comparison made using SAS statistical software (SAS Institute, Inc.). All data is presented as mean±SEM.

We claim:

1. A method for treating a subject having a disease or disorder characterized by amyloid deposit of A-beta comprising administering to the subject a therapeutically effective amount of an antibody that binds specifically to RAGE and inhibits the binding of a RAGE binding partner.

2. The method of claim 1, wherein the subject is a human subject.

3. The method of claim 1, wherein the disease or disorder is characterized by amyloid deposit of A-beta in brain.

4. The method of claim 3, wherein the disease or disorder is Alzheimer's disease.

5. The method of claim 3, wherein the disease or disorder is preclinical Alzheimer's disease.

6. The method of claim 1, wherein the antibody:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

7. The method of claim 6, wherein the antibody or RAGE-binding antibody fragment comprises:
a light chain variable region comprising CDRs of a XT-M4 light chain variable region (SEQ ID NO: 17);
a heavy chain variable region comprising CDRs of a XT-M4 heavy chain variable region sequence (SEQ ID NO: 16);
a human kappa light chain constant region; and
a human IgG1 heavy chain constant region.

8. The method of claim 7, wherein the antibody or RAGE-binding fragment thereof comprises:
a light chain variable region having the amino acid sequence of a XT-M4 light chain variable region (SEQ ID NO: 17);
a heavy chain variable region having the amino acid sequence of a XT-M4 heavy chain variable region sequence (SEQ ID NO: 16);
a human kappa light chain constant region; and
and a human IgG1 heavy chain constant region.

9. The method of claim 1, wherein the antibody is a chimeric, humanized, or human antibody.

10. The method of claim 9, wherein the chimeric or humanized antibody comprises human constant regions or constant regions derived therefrom.

11. The method of claim 1, comprising administering the antibody or RAGE-binding fragment thereof in combination with one or more agents useful for the treatment of Alzheimer's disease, to thereby elicit a synergistic therapeutic effect.

12. A method of inhibiting or reducing accumulation of amyloid deposit of A-beta in a subject, comprising administering to the subject an effective amount of an antibody that binds specifically to RAGE and inhibits the binding of a RAGE binding partner.

13. The method of claim 12, wherein the subject is a human subject.

14. The method of claim 12, comprising inhibiting or reducing accumulation of amyloid deposit of A-beta in brain.

15. The method of claim 14, wherein the accumulation of amyloid deposit of A-beta in brain is associated with Alzheimer's disease.

16. The method of claim 14, wherein the accumulation of amyloid deposit of A-beta in brain is associated with preclinical Alzheimer's disease.

17. The method of claim 12, wherein the antibody:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

18. The method of claim 17, wherein the antibody or RAGE-binding antibody fragment comprises:
a light chain variable region comprising CDRs of a XT-M4 light chain variable region (SEQ ID NO: 17);
a heavy chain variable region comprising CDRs of a XT-M4 heavy chain variable region sequence (SEQ ID NO: 16);
a human kappa light chain constant region; and
a human IgG1 heavy chain constant region.

19. The method of claim 18, wherein the antibody or RAGE-binding fragment thereof comprises:
a light chain variable region having the amino acid sequence of a XT-M4 light chain variable region (SEQ ID NO: 17);
a heavy chain variable region having the amino acid sequence of a XT-M4 heavy chain variable region sequence (SEQ ID NO: 16);
a human kappa light chain constant region; and
and a human IgG1 heavy chain constant region.

20. The method of claim 12, wherein the antibody is a chimeric, humanized, or human antibody.

21. The method of claim 20, wherein the chimeric or humanized antibody comprises human constant regions or constant regions derived therefrom.

22. The method of claim 12, comprising administering the antibody or RAGE-binding fragment thereof in combination with one or more agents useful for inhibiting or reducing of amyloid deposit of A-beta, to thereby elicit a synergistic effect.

23. A method of inhibiting or reducing neurodegeneration in a subject, comprising administering to the subject an effective amount of an antibody that binds specifically to RAGE and inhibits the binding of a RAGE binding partner.

24. The method of claim 23, wherein the subject is a human subject.

25. The method of claim 23, comprising inhibiting or reducing neurodegeneration in brain.

26. The method of claim 25, wherein the neurodegeneration is associated with Alzheimer's disease.

27. The method of claim 25, wherein the neurodegeneration is associated with preclinical Alzheimer's disease.

28. The method of claim 23, wherein the antibody:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

29. The method of claim 28, wherein the antibody or RAGE-binding antibody fragment comprises:
a light chain variable region comprising CDRs of a XT-M4 light chain variable region (SEQ ID NO: 17);
a heavy chain variable region comprising CDRs of a XT-M4 heavy chain variable region sequence (SEQ ID NO: 16);
a human kappa light chain constant region; and
a human IgG1 heavy chain constant region.

30. The method of claim 29, wherein the antibody or RAGE-binding fragment thereof comprises:
a light chain variable region having the amino acid sequence of a XT-M4 light chain variable region (SEQ ID NO: 17);
a heavy chain variable region having the amino acid sequence of a XT-M4 heavy chain variable region sequence (SEQ ID NO: 16);
a human kappa light chain constant region; and
and a human IgG1 heavy chain constant region.

31. The method of claim 23, wherein the antibody is a chimeric, humanized, or human antibody.

32. The method of claim 31, wherein the chimeric or humanized antibody comprises human constant regions or constant regions derived therefrom.

33. The method of claim 23, comprising administering the antibody or RAGE-binding fragment thereof in combination with one or more agents useful for inhibiting or reducing neurodegeneration, to thereby elicit a synergistic effect.

34. A method of inhibiting or reducing cognitive decline, or improving cognition, in a subject, comprising administering to the subject an effective amount of an antibody that binds specifically to RAGE and inhibits the binding of a RAGE binding partner.

35. The method of claim 34, wherein the subject is a human subject.

36. The method of claim 34, wherein the cognitive decline is associated with Alzheimer's disease.

37. The method of claim 34, wherein the cognitive decline is associated with preclinical Alzheimer's disease.

38. The method of claim 34, wherein the antibody:
(a) competes for binding to RAGE with an antibody selected from the group consisting of XT-H1, XT-H2, XT-H3, XT-H5, XT-H7, and XT-M4;
(b) binds to an epitope of RAGE that is bound by an antibody selected from the group consisting of XT-H1, XT-H2, XT-H3, XT-H5, XT-H7, and XT-M4;
(c) comprises one or more complementarity determining regions (CDRs) of a light chain or heavy chain of an antibody selected from the group consisting of XT-H1, XT-H2, XT-H3, XT-H5, XT-H7, and XT-M4; or
(d) is a RAGE-binding fragment of an antibody according to (a), (b) or (c).

39. The method of claim 38, wherein the antibody or RAGE-binding antibody fragment comprises:
a light chain variable region comprising CDRs of a XT-M4 light chain variable region (SEQ ID NO: 17);
a heavy chain variable region comprising CDRs of a XT-M4 heavy chain variable region sequence (SEQ ID NO: 16);
a human kappa light chain constant region; and
a human IgG1 heavy chain constant region.

40. The method of claim 39, wherein the antibody or RAGE-binding fragment thereof comprises:
a light chain variable region having the amino acid sequence of a XT-M4 light chain variable region (SEQ ID NO: 17);
a heavy chain variable region having the amino acid sequence of a XT-M4 heavy chain variable region sequence (SEQ ID NO: 16);
a human kappa light chain constant region; and
and a human IgG1 heavy chain constant region.

41. The method of claim 34, wherein the antibody is a chimeric, humanized, or human antibody.

42. The method of claim 41, wherein the chimeric or humanized antibody comprises human constant regions or constant regions derived therefrom.

43. The method of claim 34, comprising administering the antibody or RAGE-binding fragment thereof in combination with one or more agents useful for inhibiting or reducing cognitive decline, or improving cognition, to thereby elicit a synergistic effect.


SEQUENCE LISTING (64 sequences)

SEQ ID NO: 1 - protein, 404 aa, Homo sapiens
  MISC_FEATURE: Genbank Accession No. NP_001127
MAAGTAVGAWVLVLSLWGAVVGAQNITARIGEPLVLKCKGAPKKPPQRLEWKLNTGRTEAWKVLSPQGGGPWDSVARVLPNGSLFLPAVGIQDEGIFRCQAMNRNGKETKSNYRVRVYQIPGKPEIVDSASELTAGVPNKVGTCVSEGSYPAGTLSWHLDGKPLVPNEKGVSVKEQTRRHPETGLFTLQSELMVTPARGGDPRPTFSCSFSPGLPRHRALRTAPIQPRVWEPVPLEEVQLVVEPEGGAVAPGGTVTLTCEVPAQPSPQIHWMKDGVPLPLPPSPVLILPEIGPQDQGTYSCVATHSSHGPQESRAVSISIIEPGEEGPTAGSVGGSGLGTLALALGILGGLGTAALLIGVILWQRRQRRGEERKAPENQEEEEERAELNQSEEPEAGESSTGGP

SEQ ID NO: 2 - DNA, 1414 bp, Homo sapiens
  misc_feature: Genbank Accession No. NM_001136
gccaggaccctggaaggaagcaggatggcagccggaacagcagttggagcctgggtgctggtcctcagtctgtggggggcagtagtaggtgctcaaaacatcacagcccggattggcgagccactggtgctgaagtgtaagggggcccccaagaaaccaccccagcggctggaatggaaactgaacacaggccggacagaagcttggaaggtcctgtctccccagggaggaggcccctgggacagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccagaaattgtagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaacagaccaggagacaccctgagacagggctcttcacactgcagtcggagctaatggtgaccccagcccggggaggagatccccgtcccaccttctcctgtagcttcagcccaggccttccccgacaccgggccttgcgcacagcccccatccagccccgtgtctgggagcctgtgcctctggaggaggtccaattggtggtggagccagaaggtggagcagtagctcctggtggaaccgtaaccctgacctgtgaagtccctgcccagccctctcctcaaatccactggatgaaggatggtgtgcccttgccccttccccccagccctgtgctgatcctccctgagatagggcctcaggaccagggaacctacagctgtgtggccacccattccagccacgggccccaggaaagccgtgctgtcagcatcagcatcatcgaaccaggcgaggaggggccaactgcaggctctgtgggaggatcagggctgggaactctagccctggccctggggatcctgggaggcctggggacagccgccctgctcattggggtcatcttgtggcaaaggcggcaacgccgaggagaggagaggaaggccccagaaaaccaggaggaagaggaggagcgtgcagaactgaatcagtcggaggaacctgaggcaggcgagagtagtactggagggccttgaggggcccacagacagatcccatccatcagctcccttttctttttcccttgaactgttctggcctcagaccaactctctcctgtataatctctctcctgtataaccccaccttgccaagctttcttctacaaccagagccccccacaatgatgattaaacacctgacacatcttga

SEQ ID NO: 3 - protein, 403 aa, Murine
  MISC_FEATURE: Genbank Accession No. NP_031451
MPAGTAARAWVLVLALWGAVAGGQNITARIGEPLVLSCKGAPKKPPQQLEWKLNTGRTEAWKVLSPQGGPWDSVAQILPNGSLLLPATGIVDEGTFRCRATNRRGKEVKSNYRVRVYQIPGKPEIVDPASELTASVPNKVGTCVSEGSYPAGTLSWHLDGKLLIPDGKETLVKEETRRHPETGLFTLRSELTVIPTQGGTTHPTFSCSFSLGLPRRRPLNTAPIQLRVREPGPPEGIQLLVEPEGGIVAPGGTVTLTCAISAQPPPQVHWIKDGAPLPLAPSPVLLLPEVGHADEGTYSCVATHPSHGPQESPPVSIRVTETGDEGPAEGSVGESGLGTLALALGILGGLGVVALLVGAILWRKRQPRREERKAPESQEDEEERAELNQSEEAEMPENGAGGP

SEQ ID NO: 4 - DNA, 1348 bp, Murine
  misc_feature: Genbank Accession No. NM_007425.1
gcaccatgccagcggggacagcagctagagcctgggtgctggttcttgctctatggggagctgtagctggtggtcagaacatcacagcccggattggagagccacttgtgctaagctgtaagggggcccctaagaagccgccccagcagctagaatggaaactgaacacaggaagaactgaagcttggaaggtcctctctccccagggaggcccctgggacagcgtggctcaaatcctccccaatggttccctcctccttccagccactggaattgtcgatgaggggacgttccggtgtcgggcaactaacaggcgagggaaggaggtcaagtccaactaccgagtccgagtctaccagattcctgggaagccagaaattgtggatcctgcctctgaactcacagccagtgtccctaataaggtggggacatgtgtgtctgagggaagctaccctgcagggacccttagctggcacttagatgggaaacttctgattcccgatggcaaagaaacactcgtgaaggaagagaccaggagacaccctgagacgggactctttacactgcggtcagagctgacagtgatccccacccaaggaggaaccacccatcctaccttctcctgcagtttcagcctgggccttccccggcgcagacccctgaacacagcccctatccaactccgagtcagggagcctgggcctccagagggcattcagctgttggttgagcctgaaggtggaatagtcgctcctggtgggactgtgaccttgacctgtgccatctctgcccagccccctcctcaggtccactggataaaggatggtgcacccttgcccctggctcccagccctgtgctgctcctccctgaggtggggcacgcggatgagggcacctatagctgcgtggccacccaccctagccacggacctcaggaaagccctcctgtcagcatcagggtcacagaaaccggcgatgaggggccagctgaaggctctgtgggtgagtctgggctgggtacgctagccctggccttggggatcctgggaggcctgggagtagtagccctgctcgtcggggctatcctgtggcgaaaacgacaacccaggcgtgaggagaggaaggccccggaaagccaggaggatgaggaggaacgtgcagagctgaatcagtcagaggaagcggagatgccagagaatggtgccgggggaccgtaagagcacccagatcgagcctgtgtgatggccctagagcagctcccccacattccatcccaattcctccttgaggcacttccttctccaaccagagcccacatgatccatgctgagtaaacatttgatacggc

SEQ ID NO: 5 - protein, 8 aa, Artificial
  Streptavidin tag sequence
WSHPQFEK

SEQ ID NO: 6 - DNA, 1250 bp, Baboon
  CDS (20)..(1231)
gaccctggaaggaagcaggatggcagccggagcagcagttggagcctgggtgctggtcctcagtctgtggggggcagtagtaggtgctcaaaacatcacagcccggatcggtgagccactggtgctgaagtgtaagggggcccccaagaaaccaccccagcagctggaatggaaactgaatacaggccggacagaagcttggaaggtcctatctccccagggaggcccctgggatagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccggaaattatagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaagagaccaggagacaccctgagacggggctcttcacactgcagtcggagctaatggtgaccccagcccggggaggaaatccccatcccaccttctcctgtagcttcagcccaggccttccccgacgccgggccttgcacacagcccctatccagccccgtgtctgggagcctgtgcctctggaggaggtccaattggtggtagagccagaaggtggagcagtagctcctggtggaaccgtaaccctgacctgtgaagtccctgcccagccctctcctcagatccactggatgaaggatggtgtgcccttacccctttcccccagccctgtgctgatcctccctgagatagggcctcaggaccagggaacctacaggtgtgtggccacccatcccagccacgggccccaggaaagccgtgctgtcagcatcagcatcatcgaaccaggcgaggaggggccaactgcaggctctgtgggaggatcagggccaggaactctagccctggccctggggatcctgggaggcctggggacagccgctctgctcattggggtcatcttgtggcaaaggcggcgacgccaacgagaggagaggaaggcctcagaaaaccaggaggaagaggaggagcgtgcagagctgaatcagtcggaggaacctgaggcaggcgagagtggtactggagggccttgaggggcccacagacagatcc

SEQ ID NO: 7 - protein, 403 aa, Baboon
MAAGAAVGAWVLVLSLWGAVVGAQNITARIGEPLVLKCKGAPKKPPQQLEWKLNTGRTEAWKVLSPQGGPWDSVARVLPNGSLFLPAVGIQDEGIFRCQAMNRNGKETKSNYRVRVYQIPGKPEIIDSASELTAGVPNKVGTCVSEGSYPAGTLSWHLDGKPLVPNEKGVSVKEETRRHPETGLFTLQSELMVTPARGGNPHPTFSCSFSPGLPRRRALHTAPIQPRVWEPVPLEEVQLVVEPEGGAVAPGGTVTLTCEVPAQPSPQIHWMKDGVPLPLSPSPVLILPEIGPQDQGTYRCVATHPSHGPQESRAVSISIIEPGEEGPTAGSVGGSGPGTLALALGILGGLGTAALLIGVILWQRRRRQREERKASENQEEEEERAELNQSEEPEAGESGTGGP

SEQ ID NO: 8 - DNA, 1250 bp, Cynomologus Monkey
  CDS (20)..(1231)
gaccctggaaggaagcaggatggcagccggagcagcagttggagcctgggtgctggtcctcagtctgtggggggcagtagtaggtgctcaaaacatcacagcccggatcggtgagccactggtgctgaagtgtaagggggcccccaagaaaccaccccagcagctggaatggaaactgaatacaggccggacagaagcttggaaggtcctatctccccagggaggcccctgggatagtgtggctcgtgtccttcccaacggctccctcttccttccggctgtcgggatccaggatgaggggattttccggtgccaggcaatgaacaggaatggaaaggagaccaagtccaactaccgagtccgtgtctaccagattcctgggaagccggaaattatagattctgcctctgaactcacggctggtgttcccaataaggtggggacatgtgtgtcagagggaagctaccctgcagggactcttagctggcacttggatgggaagcccctggtgcctaatgagaagggagtatctgtgaaggaagagaccaggagacaccctgagacggggctcttcacactgcagtcggagctaatggtgaccccagcccggggaggaaatccccatcccaccttctcctgtagcttcagcccaggccttccccgacgccgggccttgcacacagcccctatccagccccgtgtctgggagcctgtgcctctggaggaggtccaattggtggtagagccagaaggtggagcagtagctcctggtggaaccgtaaccctgacctgtgaagtccctgcccagccctctcctcaaatccactggatgaaggatggtgtgcccttacccctttcccccagccctgtgctgatcctccctgagatagggcctcaggaccagggaacctacaggtgtgtggccacccatcccagccacgggccccaggaaagccgtgctgtcagcatcagcatcatcgaaccaggcgaggaggggccaactgcaggctctgtgggaggatcagggccaggaactctagccctggccctggggatcctgggaggcctggggacagccgctctgctcattggggtcatcttgtggcaaaggcggcgacgccaaggagaggagaggaaggcctcagaaaaccaggaggaagaggaggagcgtgcagagctgaatcagtcggaggaacctgaggcaggcgagagtggtactggagggccttgaggggcccacagacagatcc

SEQ ID NO: 9 - protein, 403 aa, Cynomologus Monkey
MAAGAAVGAWVLVLSLWGAVVGAQNITARIGEPLVLKCKGAPKKPPQQLEWKLNTGRTEAWKVLSPQGGPWDSVARVLPNGSLFLPAVGIQDEGIFRCQAMNRNGKETKSNYRVRVYQIPGKPEIIDSASELTAGVPNKVGTCVSEGSYPAGTLSWHLDGKPLVPNEKGVSVKEETRRHPETGLFTLQSELMVTPARGGNPHPTFSCSFSPGLPRRRALHTAPIQPRVWEPVPLEEVQLVVEPEGGAVAPGGTVTLTCEVPAQPSPQIHWMKDGVPLPLSPSPVLILPEIGPQDQGTYRCVATHPSHGPQESRAVSISIIEPGEEGPTAGSVGGSGPGTLALALGILGGLGTAALLIGVILWQRRRRQGEERKASENQEEEEERAELNQSEEPEAGESGTGGP

SEQ ID NO: 10 - DNA, 1220 bp, Rabbit
  CDS (18)..(1196)
actagactagtcggaccatggcagcaggggcagcggccggagcctgggtgctggtcttcagtctgtggggggcagcagtaggtggtcagaacatcacagcccggattggggagccgctggtgctgaagtgtaagggggcccccaagaagccaccccagcagctggaatggaaactgaacacaggcaggacagaagcttggaaagtcctttctccccagggaggctcatgggacagtgtggcccgtgtcctccccaatggctccctcctccttccggctgttgggatccaggatgaggggactttccggtgccggacaacaaacaggaatggaaaggagaccaagtccaattaccgagtccgggtctaccagattcctgggaagccagagatcctggatcctgcctctgaactcacggccggtatccccagtaaggtggggacatgtgtgtctgatgggggatatcctctggggactctcagctggcacatggatgggaaactcctggtacctgacgggaagggagtgtctgtgaaggagcagaccaggaggcaccctgacacggggctcttcaccctgcagtcagagctgatggtgactccagccgggggaggagggcctccccccaccttctcctgtagcttcagccccggcctgccccgccgccgggcctcatacacagcccccatccagcccagtgtctgggagcctgggcccctggaggttcgcttgctggtggagccagaaggtggagcagtagctcctggtgagactgtgaccctgacctgcgaagctcctgcccagccccctcctcaaatccactggatgaaggatggtatgtccctacccctgccccccagccccgtcctgctcctccctgaggtggggcctcaagatgaggggacttacagctgcgtggccacccatcccaaccgtgggccccaggaaagccttcccatcagcatcagtgtcggctctgagggtggctcaggcctggggactctagctctggccctggggatcctgggaggcctgggaacagctgccctgcttgtcggagtcatcctgtggcgaaggcggaaacgccaaggagagcagaggaaagtccctgaaaaccaggaggacgaggaggaacgcacagaactgcatcagtcggaggctcgggaggcgatggagagcggtacaggagagccctgaatagtttagcggccgcattcttat

SEQ ID NO: 11 - protein, 392 aa, Rabbit
MAAGAAAGAWVLVFSLWGAAVGGQNITARIGEPLVLKCKGAPKKPPQQLEWKLNTGRTEAWKVLSPQGGSWDSVARVLPNGSLLLPAVGIQDEGTFRCRTTNRNGKETKSNYRVRVYQIPGKPEILDPASELTAGIPSKVGTCVSDGGYPLGTLSWHMDGKLLVPDGKGVSVKEQTRRHPDTGLFTLQSELMVTPAGGGGPPPTFSCSFSPGLPRRRASYTAPIQPSVWEPGPLEVRLLVEPEGGAVAPGETVTLTCEAPAQPPPQIHWMKDGMSLPLPPSPVLLLPEVGPQDEGTYSCVATHPNRGPQESLPISISVGSEGGSGLGTLALALGILGGLGTAALLVGVILWRRRKRQGEQRKVPENQEDEEERTELHQSEAREAMESGTGEP

SEQ ID NO: 12 - DNA, 1247 bp, Rabbit
  CDS (18)..(1223)
actagactagtcggaccatggcagcaggggcagcggccggagcctgggtgctggtcttcagtctgtggggggcagcagtaggtggtcagaacatcacagcccggattggggagccgctggtgctgaagtgtaagggggcccccaagaagccaccccagcagctggaatggaaactgaacacaggcagaacagaagcttggaaagtcctttctccccagggaggctcatgggacagtgtggcccatgtcctccccaatggctccctcctccttccggctgttgggatccaggatgaggggactttccggtgccggacaacaaacaggaatggaaaggagaccaagtccaattaccgagtccgggtctaccagattcctgggaagccagagatcctggatcctgcctctgaactcacggccggtatccccagtaaggtggggacatgtgtgtctgatgggggatatcctctggggactctcagctggcacatggatgggaaactcctggtacctgacgggaagggagtgtctgtgaaggagcagaccaggaggcatcctgacacggggctcttcaccctgcagtcagagctgatggtgactccagctgggggaggagggcctccccccaccttctcctgtagcttcagccccggcctaccccgccgccgggcctcatacacagcccccatccagcccagtgtctgggagcctgggcccctggaggttcgcttgctggtggagccagaaggtggagcagtagctcctggtgagactgtgaccctgacctgtgaagctcctgcccagccccctcctcaaatccactggatgaaggatggtatgtccctacccctgccccccagccccgtcctgctcctccctgaggtggggcctcaagatgaggggacttacagctgcgtggccacccatcccaaccgtgggccccaggaaagccttcccatcagcatcagtgtcgagacaggcgaggatgggccgactgcaggctctgagggtggctcaggcctggggactctagctctggccctggggatcctgggaggcctgggaacagctgccctgcttgtcggagtcatcctgtggcgaaggcggaaacgccaaggagagcagaggaaagtccccgaaaaccaggaggacgaggaggaacgcacagaactgcatcagtcggaggctcgggaggcgatggagagcggtacaggagagccctgaatagtttagcggccgcattcttat

SEQ ID NO: 13 - protein, 401 aa, Rabbit
MAAGAAAGAWVLVFSLWGAAVGGQNITARIGEPLVLKCKGAPKKPPQQLEWKLNTGRTEAWKVLSPQGGSWDSVAHVLPNGSLLLPAVGIQDEGTFRCRTTNRNGKETKSNYRVRVYQIPGKPEILDPASELTAGIPSKVGTCVSDGGYPLGTLSWHMDGKLLVPDGKGVSVKEQTRRHPDTGLFTLQSELMVTPAGGGGPPPTFSCSFSPGLPRRRASYTAPIQPSVWEPGPLEVRLLVEPEGGAVAPGETVTLTCEAPAQPPPQIHWMKDGMSLPLPPSPVLLLPEVGPQDEGTYSCVATHPNRGPQESLPISISVETGEDGPTAGSEGGSGLGTLALALGILGGLGTAALLVGVILWRRRKRQGEQRKVPENQEDEEERTELHQSEAREAMESGTGEP

SEQ ID NO: 14 - protein, 402 aa, Rattus norvegicus
  MISC_FEATURE: Genbank No. NP_445788
MPTGTVARAWVLVLALWGAVAGGQNITARIGEPLMLSCKGAPKKPTQKLEWKLNTGRTEAWKVLSPQGDPWDSVARILPNGSLLLPAIGIVDEGTFRCRATNRLGKEVKSNYRVRVYQIPGKPEIVNPASELTANVPNKVGTCVSEGSYPAGTLSWHLDGKPLIPDGKGTVVKEETRRHPETGLFTLRSELTVTPAQGGTTPTYSCSFSLGLPRRRPLNTAPIQPRVREPLPPEGIQLLVEPEGGTVAPGGTVTLTCAISAQPPPQIHWIKDGTPLPLAPSPVLLLPEVGHEDEGIYSCVATHPSHGPQESPPVNIRVTETGDEGQAAGSVDGSGLGTLALALGILGGLGIAALLIGAILWRKRQPRLEERKAPESQEDEEERAELNQSEEAEMPENGAGGP

SEQ ID NO: 15 - DNA, 5301 bp, baboon
ttttttaaaaactactatttgaaaattggagggggaagagtgggaagggagttattgccaaatatgttaaatatgggttggggtgcttgtatatgtatcttcctcaatttccccagaaatgaggtatctttttgtcacaccaaaatcaagtggtagggagagggaggaggttgcaaaaagccaagtgtgggggaaaagtaaaatcaacactgtcccatcctcagccctaaaccctaccatctgatcccctcagacattctcaggattttgcaagactgtcagagtggggaacccctcccattaaagatctgggcaggactggggacaggttggaagtgtgatgggtgggggggtgggaggcatgggctgggggcagttctctcctcacttgtaaacttgtgtagtttcacagaaaaaaaacaaaatgcagttttaaataaagaagtttctttttttcctgggtttagttgagaatttttttcaaaaaacatgagaaaccccagaaaaaaaaatatatattttctttcacgaagctccaaacaggtttctctcctgtcccccagccttgccttcacgatgcagtcccaattgcacccttgcaaacaacagtctggcctcaacgctattgatgcaaccttgcccagtcaacatggggctccagtgggtcaccaggcagccctgatggactgatggaataaataggatagggggctctgagggaatgagaccctagagggtacactccccatcccccagggaagtgactgtgtccagaggctggtagtgcccaggggtggggtgataattatttctctagtacctgaaggactcttgtcccaaaggcatgaattcctagcattccctgtgacaagatgactgaaagatgggggctggagagagggtacaggccccacctagggcggaggccacagcgcggagaggggcagacagagctatgagcctggaaggaagcaggatggcagccggagcagcagttggagcctgggtgctggtcctcagtctgtggggtgagccactccccccacccactgacccttcctacagaaagcacttgaaccccataccccaattgccctagaacttttcccagaacccagggaactgcttttcaaggtcccgcacgcaccctgtccaaattttgttagccctcattaccttcctgcccctctaccacgatgctgtctcccaggggcagtagtaggtgctcaaaacatcacagcccggatcggtgagccactggtgctgaagtgtaagggggcccccaagaaaccaccccagcagctggaatggaaactggtaagtggggatcctgttgcagcttcccaacttccagggagaccagcaatgattcggatccccatcactctgcctcacagtactttcccaaaggccttgcactgtttaggccctgcttctctgcttctagaatacaggccggacagaagcttggaaggtcctatctccccagggaggcccctgggatagtgtggctcgtgtccttcccaacggctccctcttccttccggctgtcgggatccaggatgaggggattttccggtgccaggcaatgaacaggaatggaaaggagaccaagtccaactaccgagtccgtgtctaccgtaagaattccagggccttctccaaggccccctctcttatctcagaaaaagccttcaaccctagccttggcccatgagggcctctgacttccactggccccatttccacacacagagtttgagaaccttcacaattacagcctctgattggatttttccttcttcagagattcctgggaagccggaaattatagattctgcctctgaactcacggctggtgttcccaacaaggtagtgaaagaaaggagaagtagaaaatggtcctgtgaacaggaggcgagtgtgtgtgggtgtgtggcatctctcattttcaaaggattctgaggtcaccactctttccccaggtggggacatgtgtgtcagagggaagctaccctgcagggactcttagctggcacttggatgggaagcccctggtgcctaatgagaagggtgagtccgaaggtgcccgccaagctgccttctccctgatctcactcccacacccaccctgggataatttgtcttatcctcctaccataggagtatctgtgaaggaagagaccaggagacaccctgagacggggctcttcacactgcagtcggagctaatggtgaccccagcccggggaggaaatccccatcccaccttctcctgtagcttcagcccaggccttccccgacgccgggccttgcacacagcccctatccagccccgtgtctggggtgagcagaggtggggagggctccaagctcatgtgagtgcattctggaagtcggacccttagggaaagagggagtcaagcccatggccactgggatcactcacaagtgtaactctccacctcataacccttccaactcccagagcctgtgcctctggaggaggtccaattggtggtagagccagaaggtggagcagtagctcctggtggaaccgtaaccctgacctgtgaagtccctgcccagccctctcctcagatccactggatgaaggatgtgagtgacctggagagaggggctgggaggtagggtgaaccataactagcaacagggagggcagagggctaacgagggaaaggcaggctaggagctgaggaggaagagagggtatttgaagatgtggagacaaaaagataagagttttgaaatagtctcctctccccttcccccaccagggtgtgcccttacccctttcccccagccctgtgctgatcctccctgagatagggcctcaggaccagggaacctacaggtgtgtggccacccatcccagccacgggccccaggaaagccgtgctgtcagcatcagcatcatcggtgagacctctctccaagccctacagaccctggggctagggtgcaggatagcacaggctctaatttcctgccccattctggccttacccccaagagccagcccacctctccctccgtgcacccacacccaaacctcccctgccccactcaaattctgccaagagagcagccaagcctctcccttcttccctctgaactaaaaaaagggaaagacggctgggcgcagtggctcacgcctgtaatcccaacactttgggaggctgaggccggtggatcacctgaggttgggagttcaagaccagtctgaccaacatggaggaaccccatttctactaaaaatacaaaattagccagttgtggtggcacgtgcctgtaatcccagctacttgggaggctgagacaggaaaatcacttgaacccgggaggcgtaagttgcggtgagccaagatcctgccattgcatgccagcctgggcaacaagagcgaaactccatctcaaaaaaaaaaaaaaaagaaagggaaagactccactggagctcccactaaataaccctctctcaacccgaagtcttcctttctgactggatccaactttgtcttccagaaccaggcgaggaggggccaactgcaggtgaggggttcgagaaagtcagggaagcagaagatagcccccaacacatgtgactgcggggatggtcaacaagaaaggaatggtggccgggcgcggtggctcaagcctgtaatcccagcactttgggaggccgagatgggcggatcacgaggtcaggagatcgagaccatcctggctaacacagtaaaaccccatctctaccaaaaaaaaatacaaaaaactagccgggcgacgtggcgggcgcctgtagtcccagctactcgggaggctgaggcaggagaatggtgtaaacccgggaggcggagcttgcagtgagctgagatccggccactgcactccagcctgggcaacagagccagactccatctcaaaaaaaaaaaaaaagaaagaaaggaatggtgagtggtgggggctgtgctctcaattttccctgtctccctacaggctctgtgggaggatcagggccaggaactctagccctggccctggggatcctgggaggcctggggacagccgctctgctcattggggtcatcttgtggcaaaggcggcgacgccaacgagaggagaggtgagtggagaaagccagactcctcagacctagggcttccaggcagcaagtgaagagggatggggggtggaacgacaacgtgcagcattctccacaatcttcctcctcaggaaggcctcagaaaaccaggaggaagaggaggagcgtgcagagctgaatcagtcggaggaacctgaggcaggcgagagtggtactggagggccttgaggggcccacagagagatcccatccatcagctcccttttcttcttcccttgaactgttctggccccagaccaactctctcctgtataaccccaccttgccaaactttcttctacaaccagagccccccacaatgatgagtaaacacctgacacatcttgctcttgtgtgtctgtgtatatgtgtgtgagacacaacctcacccctacacccttgagggccctgaaggaaagggactcacccccacactgcaccaaacatctactcaagttggggagaagatgcttctgtcaagagagggagggaggaaggtggggggcaaacttgggaagagatcccatcaatacatttcaccttttttattgaatttgtattaaaggaggtagtaagggggaggaagcacttaagagtcagaacccatattagactctggggagtgaaaaattaaattaaatcaataagatgggagtgggggaagagtcagagggagctttgcccccctttcaagatcaaatcaagaaatcagggaaagcaaagacttagaagagaagaaagacattctctcaatccatcctccttccccagggcagagaattataatgttactgagtgagcttctgagcagaaggctctcccatctatgcacagacttcactcctcctccccaagctttcctggagaatgtccagggctggccttagccaacagaaatagagaggtcaagggggtccatgagtaaggaagggtcagcagggacccccaatactgattctcctctggctggaggtgggcaggaaggagacatagctcaaatactgagcagccaaaaaaagaagaagatggcgagaaacaggaagagggaatcctgccagctggaggccgggtgaccctgtcccagatccacacctgtgggagagaggaaagctgtggaagcatatgcttctaggctgggaggg

SEQ ID NO: 16 - protein, 119 aa, Rat
  MISC_FEATURE: XT-M4 VH
  MISC_FEATURE: Variable Heavy Region
EVQLVESGGGLVQPGRSLKLSCVVSGFTFNNYWMTWIRQTPGKGLEWVASINNSGDNTYYPDSVKDRFTISRDNAKSTLYLQMNSLRSEDTATYYCTRGGDITTGFDYWGQGVMVTVSS

SEQ ID NO: 17 - protein, 107 aa, Rat
  MISC_FEATURE: XT-M4 VL
  MISC_FEATURE: Variable Light Region
DIQMTQSPSSMSVSLGDTITITCRASQDVGIYVNWFQQKPGKSPRRMIYRATNLADGVPSRFSGSRSGSIYSLTISSLESEDVADYHCLEFDEHPLTFGSGTKVEIK

SEQ ID NO: 18 - protein, 119 aa, Murine
  MISC_FEATURE: XT-H1 VH
  MISC_FEATURE: Variable Heavy Region
QVQLKESGPGLVAPSQSLSITCTISGFSITSYGVHWVRQPPGKGLEWLVVIWSDGRTTYNSTLKSRLSISKDNSKSQVFLKMNSLQTDDTAMYYCVRHGGYFPYAMDYWGQGTSVTVSS

SEQ ID NO: 19 - protein, 106 aa, Murine
  MISC_FEATURE: XT-H1 VL
  MISC_FEATURE: Variable Light Region
DIQMTQSPSSLSTSLGGKVTITCKASQDINKFIAWYQHTPGKGPRLFIYYTSTLQPGIPSRFSGNGSGRDYSFSISNLEPEDIATYYCLQYDNMYTFGGGTKLEIK

SEQ ID NO: 20 - protein, 119 aa, Murine
  MISC_FEATURE: XT-H5 VH
  MISC_FEATURE: Variable Heavy Region
EVLLQQSGPELVKPGASVKISCKASGYTFTDYNMDWVKQSHGKSLEWIGDINPDNGGTIYKQKFKGKATLTVDKSSSTAYMELRSLTSEDTAVYYCTTHDHYYYALDYWGQGTSVTVSS

SEQ ID NO: 21 - protein, 117 aa, Murine
  MISC_FEATURE: XT-H2_VH
  MISC_FEATURE: Variable Heavy Region
QVQLQQSGAELAKPGASVKMSCKASGYTFTTYWMHWVKQRPGQGLYWIGYINPSTGYTEYNQKFKDKATLTADKSSSTAYMQLSSLTSEDSAVYYCARWAGYTIDYWGQGTSVTVSS

SEQ ID NO: 22 - protein, 112 aa, Murine
  MISC_FEATURE: XT-H2-VL
  MISC_FEATURE: Variable Light Region
DVVLTQTPLSLPVNIGDQASISCKSTKSLLNSDGFTYLDWYLQKPGQSPQLLIYLVSDRFSGVPDRFSGSGSGTDFTLTISRVEAEDLGVYYCFQSNSLPLTFGSGTKLEIK

SEQ ID NO: 23 - protein, 111 aa, Murine
  MISC_FEATURE: XT-H5 VL
  MISC_FEATURE: Variable Light Region
DIVLTQSPASLAVSLGQRATISCRASESVDNYGISFMNWFQQKPGQPPRLLIYTVSNQGSGVPARFSGSGSGTDFSLNIHPMEEDDTAMYFCQQSKEVPWTFGGGTKLEIK

SEQ ID NO: 24 - protein, 116 aa, Murine
  MISC_FEATURE: XT-H3 VH
  MISC_FEATURE: Variable Heavy Region
QVQLQQPGSELVRPGASVKLSCKTSGYTFTNYWMHWVKQRHGQGLEWIGNLYPGSGRTNYDEKFKSKVTLTEDTSSSTAYMHLSNLTSEDSAVYYCTSLRRGFVYWGQGTLVTVSA

SEQ ID NO: 25 - protein, 107 aa, Murine
  MISC_FEATURE: XT-H3 VL
  MISC_FEATURE: Variable Light Region
NIVMTQSPEYMSMSFGERVTLTCKASENVGSYVSWYQQKSEQSPKLLIYGASNRYTGVPDRFTGSGSATDFTLTITSVQAEDLADYHCGQTYTYPYTFGGGTKLEIK

SEQ ID NO: 26 - protein, 116 aa, Murine
  MISC_FEATURE: XT-H7 VH
  MISC_FEATURE: Variable Heavy Region
QVQLKESGPGLVAPSQSLSITCTVSGFSLTNYGVHWVRQPPGKGLEWLGVMWAGGSTTYNSALMSRLSISKDNSKSQVFLKMNSLQTDDTAMYYCARYGNYAMDYWGQGTSVTVSS

SEQ ID NO: 27 - protein, 106 aa, Murine
  MISC_FEATURE: XT-H7 VL
  MISC_FEATURE: Variable Light Region
DIQMTQSPSSLSASLGGKVTITCKASQDIYKYIAWYQHKPGKGPRLLIHYTSTLQPGIPSRFSGSGSGRDYSFSISNLEPEDIATYYCLRYDNLYTFGGGTKLEIK

SEQ ID NO: 28 - protein, 117 aa, Artificial
  Humanized XT-H2_hVH_V2.0
  MISC_FEATURE: Amino acid sequences of humanized heavy chain variable regions
QVQLVQSGAEVKKPGASVKVSCKASGYTFTTYWMHWVRQRPGQGLYWIGYINPSTGYTEYNQKFKDRVTMTADTSISTAYMELSRLRSDDTAVYYCARWAGYTIDYWGQGTLVTVSS

SEQ ID NO: 29 - protein, 117 aa, Artificial
  Humanized XT-H2_hVH_V2.7
  MISC_FEATURE: Amino acid sequences of humanized heavy chain variable regions
QVQLVQSGAEVKKPGASVKVSCKASGYTFTTYWMHWVRQAPGQGLEWMGYINPSTGYTEYNQKFKDRVTMTRDKSISTAYMELSRLRSDDTAVYYCARWAGYTIDYWGQGTLVTVSS

SEQ ID NO: 30 - protein, 117 aa, Artificial
  XT-H2_hVH_V4.0
  MISC_FEATURE: Amino acid sequences of humanized heavy chain variable regions
EVQLVESGGGLVQPGGSLRLSCAASGYTFTTYWMHWVRQAPGKGLEWVAYINPSTGYTEYNQKFKDRFTISRDNAKNSLYLQMNSLRAEDTAVYYCARWAGYTIDYWGQGTLVTVSS

SEQ ID NO: 31 - protein, 120 aa, Artificial
  Humanized XT-H2_hVH_V4.1
  MISC_FEATURE: Amino acid sequences of humanized heavy chain variable regions
EVQLVESGGGLVQPGGSLRLSCAASGYTFTTYWMHWMSWVRQAPGKGLYWVAYINPSTGYTEYNQKFKDRFTISADKAKSSLYLQMNSLRAEDTAVYYCARWAGYTIDYWGQGTLVTVSS

SEQ ID NO: 32 - protein, 112 aa, Artificial
  Humanized XT-H2_hVL_V2.0
  MISC_FEATURE: Amino acid sequence of humanized light chain variable regions
DVVLTQTPLSLSVTPGQPASISCKSTKSLLNSDGFTYLDWYLQKPGQSPQLLIYLVSDRFSGVPDRFSGSGSGTDFTLKISRVEAEDVGVYYCFQSNSLPLTFGGGTKVEIK

SEQ ID NO: 33 - protein, 112 aa, Artificial
  Humanized XT-H2_hVL_V3.0
  MISC_FEATURE: Amino acid sequence of humanized light chain variable regions
DIVMTQSPDSLAVSLGERATINCKSTKSLLNSDGFTYLDWYQQKPGQPPKLLIYLVSDRFSGVPDRFSGSGSGTDFTLTISSLQAEDVAVYYCFQSNSLPLTFGGGTKVEIK

SEQ ID NO: 34 - protein, 112 aa, Artificial
  Humanized XT-H2_hVL_V4.0
  MISC_FEATURE: Amino acid sequence of humanized light chain variable regions
DIQMTQSPSSLSASVGDRVTITCKSTKSLLNSDGFTYLDWYQQKPGKAPKLLIYLVSDRFSGVPSRFSGSGSGTDFTLTISSLQPEDFATYYCFQSNSLPLTFGGGTKVEIK

SEQ ID NO: 35 - protein, 112 aa, Artificial
  Humanized XT-H2_hVL_V4.1
  MISC_FEATURE: Amino acid sequence of humanized light chain variable regions
DVQMTQSPSSLSASVGDRVTITCKSTKSLLNSDGFTYLDWYQQKPGKAPKLLIYLVSDRFSGVPSRFSGSGSGTDFTLTISSLQPEDFATYYCFQSNSLPLTFGGGTKVEIK

SEQ ID NO: 36 - protein, 119 aa, Artificial
  Humanized XT-M4_hVH_V1.0
  MISC_FEATURE: Amino acid sequences of humanized heavy chain variable regions
EVQLVESGGGLVQPGGSLRLSCAASGFTFNNYWMTWVRQAPGKGLEWVASINNSGDNTYYPDSVKDRFTISRDNAKNSLYLQMNSLRSEDTAVYYCARGGDITTGFDYWGQGTLVTVSS

SEQ ID NO: 37 - protein, 119 aa, Artificial
  Humanized XT-M4_hVH_V1.1
  MISC_FEATURE: Amino acid sequences of humanized heavy chain variable regions
EVQLVESGGGLVQPGGSLRLSCAASGFTFNNYWMTWVRQAPGKGLEWVASIDNSGDNTYYPDSVKDRFTISRDNAKNSLYLQMNSLRSEDTAVYYCARGGDITTGFDYWGQGTLVTVSS

SEQ ID NO: 38 - protein, 119 aa, Artificial
  Humanized XT-M4_hVH_V2.0
  MISC_FEATURE: Amino acid sequences of humanized heavy chain variable regions
EVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSS

SEQ ID NO: 39 - protein, 107 aa, Artificial
  Humanized XT-M4_hVL_V2.4 \ (G66R)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSRSGTDFTLTISSLQPEDFATYYCLEFDEHPLTFGGGTKVEIK

SEQ ID NO: 40 - protein, 107 aa, Artificial
  Humanized XT-M4_hVL_V2.5 \ (D70I)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSGSGTIFTLTISSLQPEDFATYYCLEFDEHPLTFGGGTKVEIK

SEQ ID NO: 41 - protein, 107 aa, Artificial
  Humanized XT-M4_hVL_V2.6 \ (T69S)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSGSGSDFTLTISSLQPEDFATYYCLEFDEHPLTFGGGTKVEIK

SEQ ID NO: 42 - protein, 107 aa, Artificial
  Humanized XT-M4_hVL_V2.7 \ (L46R)
  MISC_FEATURE: Amino acid sequences of humanized light chain variable regions
DIQMTQSPSSLSASVGDRVTITCRASQDVGIYVNWYQQKPGKAPKRLIYRATNLADGVPSRFSGSGSGTDFTLTISSLQPEDFATYYCLEFDEHPLTFGGGTKVEIK

SEQ ID NO: 43 - protein, 107 aa, Artificial
  Humanized XT-M4_hVL_V2.8
  MISC_FEATURE: Amino acid sequences of humanized light chain variable regions
DIQMTQSPSSLSASVGDRVTITCRASQDVGIYVNWYQQKPGKAPKLMIYRATNLADGVPSRFSGSGSGTDFTLTISSLQPEDFATYYCLEFDEHPLTFGGGTKVEIK

SEQ ID NO: 44 - protein, 107 aa, Artificial
  Humanized XT-M4_hVL_V2.9 \ (F71Y)
  MISC_FEATURE: Amino acid sequences of humanized light chain variable regions
DIQMTQSPSSLSASVGDRVTITCRASQDVGIYVNWYQQKPGKAPKLLIYRATNLADGVPSRFSGSGSGTDYTLTISSLQPEDFATYYCLEFDEHPLTFGGGTKVEIK

SEQ ID NO: 45 - protein, 107 aa, Artificial
  Humanized XT-M4_hVL_V2.10
  MISC_FEATURE: Amino acid sequences of humanized light chain variable regions
DIQMTQSPSSLSASVGDRVTITCRASQDVGIYVNWFQQKPGKAPKRLIYRATNLADGVPSRFSGSGSGTDFTLTISSLQPEDFATYYCLEFDEHPLTFGGGTKVEIK

SEQ ID NO: 46 - protein, 107 aa, Artificial
  Humanized XT-M4_hVL_V2.11
  MISC_FEATURE: Amino acid sequences of humanized light chain variable regions
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIK

SEQ ID NO: 47 - protein, 107 aa, Artificial
  Humanized XT-M4_hVL_V2.12
  MISC_FEATURE: Amino acid sequences of humanized light chain variable regions
DIQMTQSPSSLSASVGDRVTITCRASQDVGIYVNWYQQKPGKAPRRLIYRATNLADGVPSRFSGSRSGTDFTLTISSLQPEDFATYYCLEFDEHPLTFGGGTKVEIK

SEQ ID NO: 48 - protein, 107 aa, Artificial
  Humanized XT-M4_hVL_V2.13
  MISC_FEATURE: Amino acid sequences of humanized light chain variable regions
DIQMTQSPSSLSASVGDRVTITCRASQDVGIYVNWYQQKPGKAPKRLIYRATNLADGVPSRFSGSRSGTDFTLTISSLQPEDFATYYCLEFDEHPLTFGGGTKVEIK

SEQ ID NO: 49 - protein, 107 aa, Artificial
  Humanized XT-M4_hVL_V2.14
  MISC_FEATURE: Amino acid sequences of humanized light chain variable regions
DIQMTQSPSSLSASVGDRVTITCRASQDVGIYVNWFQQKPGKAPKRLIYRATNLADGVPSRFSGSRSGTDFTLTISSLQPEDFATYYCLEFDEHPLTFGGGTKVEIK

SEQ ID NO: 50 - protein, 16 aa, Artificial
  Linker
DGGGSGGGGSGGGGSS

SEQ ID NO: 51 - DNA, 783 bp, Artificial
  XT.H2_VL-VH (direct) ScFv sequence
gtggcccaggcggccggcgcgcactccgacatccagatgacccagtccccctcctccctgtccgcctccgtgggcgaccgggtgaccatcacctgcaagtccaccaagtccctgctgaactccgacggcttcacctacctggactggtatcagcagaagcctggcaaggcccctaagctgctgatctacctggtgtccgaccggttctccggcgtgccttcccggttcagcggctccggctccggcaccgacttcaccctgaccatctcctccctccagcctgaggacttcgccacctactactgcttccagtccaactccctgcctctgacctttggcggcggaacaaaggtggaaatcaaagatggcggtggatcgggcggtggtggatctggaggaggtggaagctctgaggtgcagctcgtggagtctggcggcggactggtgcagcctggcggctccctgcggctgtcctgcgccgcctccggctacaccttcaccacctactggatgcactgggtgcggcaggcccctggcaagggcctggagtgggtcgcctacatcaacccttccaccggctataccgagtacaaccagaagttcaaggaccggttcaccatctcccgggacaacgccaagaactccctgtacctccagatgaactccctgcgggccgaggacaccgccgtgtactactgcgccagatgggctggctacaccatcgactactggggccagggcaccctggtgaccgtgtcctcatctgatcaggcctcaggggcc

SEQ ID NO: 52 - DNA, 783 bp, Artificial
  XT.H2_VH-VL (direct) ScFv sequence
gtggcccaggcggccggcgcgcactccgaggtgcagctcgtggagtctggcggcggactggtgcagcctggcggctccctgcggctgtcctgcgccgcctccggctacaccttcaccacctactggatgcactgggtgcggcaggcccctggcaagggcctggagtgggtcgcctacatcaacccttccaccggctataccgagtacaaccagaagttcaaggaccggttcaccatctcccgggacaacgccaagaactccctgtacctccagatgaactccctgcgggccgaggacaccgccgtgtactactgcgccagatgggctggctacaccatcgactactggggccagggcaccctggtgaccgtgtcctcagatggcggtggatcgggcggtggtggatctggaggaggtggaagctctgacatccagatgacccagtccccctcctccctgtccgcctccgtgggcgaccgggtgaccatcacctgcaagtccaccaagtccctgctgaactccgacggcttcacctacctggactggtatcagcagaagcctggcaaggcccctaagctgctgatctacctggtgtccgaccggttctccggcgtgccttcccggttcagcggctccggctccggcaccgacttcaccctgaccatctcctccctccagcctgaggacttcgccacctactactgcttccagtccaactccctgcctctgacctttggcggcggaacaaaggtggaaatcaaatctgatcaggcctcaggggcc

SEQ ID NO: 53 - DNA, 774 bp, Artificial
  XT.M4_VH_VL ScFv sequence
gtggcccaggcggccggcgcgcactccgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctctgatggcggtggatcgggcggtggtggatctggaggaggtggaagctctgacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaagtctgatcaggcctcaggggcc

SEQ ID NO: 54 - DNA, 774 bp, Artificial
  XT.M4_VL_VH  ScFv sequence
gtggcccaggcggccggcgcgcactccgacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtggaagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctcttctgatcaggcctcaggggcc

SEQ ID NO: 55 - protein, 41 aa, Artificial
  C terminal end of M4 ScFv - VL/VH format
LRAEDTAVYYCARGGDITTGFDYWGQGTLVTVSSSDQASGA

SEQ ID NO: 56 - DNA, 123 bp, Artificial
  C terminal end of M4 ScFv VL/VH format
ctgagggccgaggataccgccgtgtactactgtgccagaggcggcgatatcaccaccggcttcgactactggggccagggcaccctggtgaccgtgtcctcttctgatcaggcctcaggggcc

SEQ ID NO: 57 - DNA, 86 bp, Artificial
  Spiking oligonucleotide for VH3-CDR3 mutagenesis of M4 VL/VH
  misc_feature (61)..(70): N is A, T, C or G
caggttgtcggcccctgaggcctgatcagaggacacggtcaccagggtgccctggccccannnnnnnnnntctggcacagtagtac

SEQ ID NO: 58 - DNA, 53 bp, Artificial
  Spiking oligonucleotide for VH3-CDR3 mutagenesis of M4 VL/VH
  misc_feature (28)..(37): N is A, T, C or G
caggttgtccagggtgccctggccccannnnnnnnnntctggcacagtagtac

SEQ ID NO: 59 - DNA, 22 bp, Artificial
  Artificial Sequence
gaccctggaaggaagcaggatg

SEQ ID NO: 60 - DNA, 27 bp, Artificial
  Artificial Sequence
ggatctgtctgtgggcccctcaaggcc

SEQ ID NO: 61 - DNA, 41 bp, Artificial
  Artificial Sequence
actagactagtcggaccatggcagcaggggcagcggccgga

SEQ ID NO: 62 - DNA, 48 bp, Artificial
  Artificial Sequence
ataagaatgcggccgctaaactattcagggctctcctgtaccgctctc

SEQ ID NO: 63 - protein, 476 aa, Artificial
  anti-RAGE scFv-Fc fusion protein
  MISC_FEATURE: huXT-M4 V2.11 scFv-Fc with wt human IgG1 Fc VL-linker-VH
DIQMTQSPSSLSASVGDRVTITCRASQDVGIYVNWFQQKPGKAPRRLIYRATNLADGVPSRFSGSRSGTDFTLTISSLQPEDFATYYCLEFDEHPLTFGGGTKVEIKDGGGSGGGGSGGGGSSEVQLVESGGGLVQPGGSLRLSCAASGFTFNNYWMTWVRQAPGKGLEWVASIDNSGDNTYYPDSVKDRFTISRDNAKNSLYLQMNSLRAEDTAVYYCARGGDITTGFDYWGQGTLVTVSSDQEPKSSDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - DNA, 1428 bp, Artificial
  Encodes anti-RAGE scFv-Fc fusion protein
  misc_feature: huXT-M4 VL V2.11-VH V2.0 wt-Fc sequence
gacatccagatgacccagtccccctcttctctgtctgcctctgtgggcgacagagtgaccatcacctgtcgggcctctcaggatgtgggcatctacgtgaactggtttcagcagaagcctggcaaggctcccaggcgcctgatctaccgggccaccaacctggccgatggcgtgccttccagattctccggctctcgctctggcaccgatttcaccctgaccatctcctccctccagcctgaggatttcgccacctactactgcctggagttcgacgagcaccctctgacctttggcggcggaacaaaggtggagatcaaggatggcggtggatcgggcggtggtggatctggaggaggtgggagctctgaggtgcagctggtggagtctggcggcggactggtgcagcctggcggctctctgagactgtcttgtgccgcctccggcttcaccttcaacaactactggatgacctgggtgaggcaggcccctggcaagggcctggagtgggtggcctccatcgacaactccggcgacaacacctactaccccgactccgtgaaggaccggttcaccatctccagggacaacgccaagaactccctgtacctccagatgaactccctgagggccgaggataccgccgtgtactactgtgccagaggcggcgatatcaccaccggcttcgactactggggccagggcaccctggtgaccgtgtcctctgatcaggagcccaaatcttctgacaaaactcacacatgtccaccgtgcccagcacctgaactcctgggtggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatccaagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa